(19) United States
(12) Patent Application Publication
(10) Pub. No.: US 2018/0292384 A1
(43) Pub. Date: Oct. 11, 2018

(54) URINE METABOLITE PROFILES IDENTIFY KIDNEY ALLOGRAFT STATUS

(71) Applicant: Cornell University, Ithaca, NY (US)

(72) Inventors: Manikkam Suthanthiran, New York, NY (US); Karsten Suhre, Doha Qatar (DE)

(21) Appl. No.: 15/577,977

(22) PCT Filed: May 27, 2016
(86) PCT No.: PCT/US16/34752

§ 371 Date: Nov. 29, 2017

Related U.S. Application Data
(60) Provisional application No. 62/168,439, filed on May 29, 2015.

(51) Int. Cl. G01N 33/493; C12Q 1/6876

(52) U.S. Cl. CPC G01N 33/493; G01N 2430/00; G01N 2400/00; C12Q 1/6876

(57) ABSTRACT

Methods and assay processes are described herein for identifying and treating subjects who have or will have dysfunction or rejection of a kidney transplant. The methods and assay procedures are noninvasive.

### CROSS REFERENCE TO RELATED APPLICATIONS

This application claims benefit of the priority filing date of U.S. Patent Application Ser. No. 62/168,439, filed May 29, 2015, the contents of which are specifically incorporated herein by reference in their entirety.

### GOVERNMENT FUNDING

This invention was made with Government support under U01 AI63589, R37 AI051652 and R37 HL087062 awarded by National Institutes of Health, K08-DK087824 awarded by the National Institutes of Diabetic and Digestive and Kidney Diseases, and KL2-TR000458 awarded by the National Center for Advancing Translational Sciences. The United States Government has certain rights in the invention.

### BACKGROUND

Kidney transplantation is the preferred treatment for patients with end stage renal disease, but acute rejection, a frequent and serious post-transplant complication, undermines realization of the full benefits of this intervention. The invasive allograft biopsy performed to diagnose acute rejection, has become safer over the years, but bleeding and graft loss can still occur following a biopsy. Sampling errors and inter-observer variability in biopsy readings pose challenges and the feasibility and cost of repeated biopsies needed to capture anti-allograft immunity are major drawbacks. Development of noninvasive biomarkers of acute rejection is therefore a major objective of the field.

The multicenter, NIH-sponsored Clinical Trials in Organ Transplantation-04 (CTOT-04) investigated whether mRNA levels in urinary cells collected at the time of biopsy are diagnostic of acute rejection and whether mRNA profiles of sequential urine specimens obtained at clinically stable time points predict the future development of acute rejection (Suthanthiran et al., N Engl J Med 369: 20-31 (2013)). Data from the CTOT-04 study demonstrated that a 3-gene signature of 18S rRNA and 18S-normalized CD3ε mRNA and IP-10 mRNA (RNA signature) in urinary cells discriminated acute cellular rejection (ACR) biopsies from biopsies without features of rejection (No Rejection biopsies). Furthermore, there was a sharp and significant rise in the diagnostic signature score during the weeks prior to an ACR biopsy (id.). However, despite the progress towards noninvasive characterization of kidney allograft status by mRNA profiling of urine from kidney graft recipients (Suthanthiran et al., N Engl J Med 369: 20-31 (2013); Hricik et al., Am J Transplant 13: 2634-2644 (2013)), further progress can be made.

### SUMMARY

The invention relates to methods of detecting acute kidney rejection in a subject by detecting urine metabolite levels in a test urinary sample from the subject. The test is noninvasive and can be used to determine whether existing problems are present or whether problems may develop in the future. Thus, one benefit of such a noninvasive test is that allograft function can be monitored and rejection can be identified prior to organ injury and graft dysfunction. Invasive biopsies, with complications such as bleeding and even death of the patient can be avoided. Preemptive anti-rejection therapy can quickly be initiated before rejection problems have progressed because acute transplant rejection can be anticipated. Such a non-invasive test can illuminate the mechanisms responsible for acute rejection. The “one size fits all” approach typically used where all transplant recipients are treated the same way can be avoided, and anti-rejection therapy can be adapted to the specific needs of the patient.

The methods provided involve detection and quantification of two to four urine metabolites: 3-sialyllactose, xanthosine, quinolinate and X-16397 from the urine of a subject with a kidney allograft. In some cases, RNA levels expressed by three different genes: 18S ribosomal RNA, CD3ε mRNA and interferon inducible protein-10 mRNA can also be detected and quantified in the urine sample from the subject.

Ratios of the concentrations or amounts of 3-sialyllactose to xanthosine and quinolinate to X-16397 in the urine can be generated and are diagnostic of whether transplant rejection is or will occur. The ratios can be converted into log values. A combined metabolite signature can be expressed as follows:

1.1164*log(3-sialyllactose/xanthosine)+0.6937*log(quinolinate/X-16397).

Quantified RNA levels can be normalized and converted into log₁₀ values. Such an RNA signature can be expressed as follows:

RNA signature=−6.1487+0.8534 log₁₀(CD3ε/18S)+0.6376 log₁₀(IP-10/18S)+1.6464 log₁₀(18S)

where:
CD3ε refers to an absolute urinary CD3ε mRNA copy number per microgram of total RNA in a subject's urine sample;
IP-10 refers to an absolute urinary IP-10 mRNA copy number per microgram of total RNA in a subject's urine sample;
18S refers to an absolute urinary 18S rRNA copy number per microgram of total RNA times 10⁻⁶ in a subject's urine sample.

A combination of the two metabolite ratios with the RNA signature provides the following composite diagnostic signature:

RNA signature+1.1164*log(3SL/X)+0.6937*log(quinolinate/X-16397).

This composite signature was diagnostic of acute cellular rejection of kidney allografts with a specificity of 84% and a sensitivity of 90%. Taken together, these results show that adding metabolite information to the RNA signature substantially improves the diagnostic utility of a urinary screen for kidney allograft problems.

This application is related to U.S. patent application Ser. No. 14/170,132, U.S. Application Publication No. 20140213533, filed Jan. 31, 2014, which is specifically incorporated herein by reference in its entirety.

### DESCRIPTION OF THE FIGURES

FIG. 1 illustrates selection of urine samples for metabolomics. From a total of 4300 urine samples prospectively collected from the 485-kidney allograft recipients (patients) enrolled in the parent CTOT-04 study, 1518 urine samples were selected for metabolite analysis to include the following urine samples: (1) all biopsy-matched urine samples, 298 samples matched to 298 kidney allograft biopsies performed in 190 patients (urine samples collected from 3 days before to 1 day after the biopsy); (2) all 808 sequential samples from 112 patients that preceded a first biopsy classified using Banff classification schema as acute cellular rejection, antibody-mediated rejection, borderline changes, or other, and sequential samples that preceded No Rejection biopsies; and (3) all 412 sequential samples from 40 patients with stable graft function and who had at least 10 sequential samples collected in the first 400 days of transplantation and with sufficient RNA for urinary cell mRNA profiling. The kidney allograft recipients designated as patients with stable graft function did not undergo biopsy during the 400 days of transplantation and met the following additional criteria: (i) average serum creatinine less than or equal to 2.0 mg per deciliter [180 micromole per liter] at 6, 9 and 12 months following transplantation, (ii) no treatment for acute rejection, and (iii) no evidence of cytomegalovirus (CMV) or polyomavirus type BK (BKV) infection. Among the 1518 urine samples selected for metabolomics, one biopsy-matched urine sample from a patient with a No Rejection biopsy result was excluded from further analysis because of failed osmolality measurements, and one non-biopsy associated sample from a patient with other biopsy findings did not contain sufficient cell free supernatant for non-targeted metabolite analysis. After exclusion of these two samples, high-quality metabolite data from 1516 samples collected from 241 kidney patients were available for data analysis. The number of patients in the three categories listed under the metabolomics exceeds 241 unique patients because several patients had multiple urine samples and contributed urine samples to more than one category that is the same patient contributing biopsy matched urine sample as well as sequential urine samples and thereby counted in each category. A total of 2782 urine samples collected during the parent CTOT-04 study were excluded from non-targeted metabolite analysis because: (1) the urine samples were not matched to biopsy specimens or were collected following a biopsy (1074 specimens from 187 patients), (2) had unstable allograft function and no biopsy or were lost to follow up (272 urine samples from 63 patients) or (3) had stable graft function but had less than 10 sequential samples collected in the first 400 days of transplantation; urine samples collected after 400 days and with insufficient RNA for mRNA profiling are also included in this group of 1436 specimens from 180 patients. The number of patients in the three categories excluded from metabolomics exceeds 243 unique patients because several patients had multiple urine samples and contributed urine samples to more than one category and thereby counted in each category. The boxes to the left denote urine samples included for urine metabolomics analysis while the boxes to the right denote urine samples excluded from urine metabolomics analysis.

FIG. 2A-2E shows Bean plots of metabolite ratios un urine samples collected from kidney graft recipients prior to kidney transplant biopsies, illustrating the specificity and sensitivity of signature scores in urine samples from patients with kidney allograft biopsies showing either acute cellular rejection (ACR, shading to the right of each plot; Light Red in the original) or no rejection (Normal, shading to the left of each plot; Light Blue in the original). FIG. 2A graphically illustrates the log ratio of 3SL/X at various time periods (in days) after kidney biopsy. As illustrated, the cut-off between samples exhibiting acute cellular rejection and samples exhibiting no rejection is about 0.59836. FIG. 2B graphically illustrate the combined metabolite signature at various time periods (in days) after kidney biopsy. As illustrated, the cut-off between samples exhibiting acute cellular rejection and samples exhibiting no rejection is about 0.4271. FIG. 2C graphically illustrates the log score of the 18S rRNA normalized measures of CD3 mRNA, IP-10 mRNA and 18S rRNA signature (RNA signature) at various time periods (in days) after kidney biopsy. As illustrated, the cut-off between samples exhibiting acute cellular rejection and samples exhibiting no rejection is about negative 1.563099 (i.e., −1.563099). FIG. 2D graphically illustrates a combination of the log ratio 3SL/X and the RNA signature at various time periods (in days) after kidney biopsy. As illustrated, the cut-off between samples exhibiting acute cellular rejection and samples exhibiting no rejection is about negative 0.8815479 (i.e., −0.8815479). FIG. 2E graphically illustrates the composite signature (i.e., the combined metabolite signature and the RNA signature) at various time periods (in days) after kidney biopsy. As illustrated, the cut-off between samples exhibiting acute cellular rejection and samples exhibiting no rejection is about negative 0.5095 (i.e., about −0.5095). The day of kidney biopsy was designated as day 0 for all analyses and data from samples collected up to 365 days prior to biopsy is shown. Scores of metabolite signatures and the RNA signature in 159 urine samples matched to 159 No Rejection biopsies and 39 samples matched to 39 ACR biopsies (biopsy matched urine samples collected 3 days before to 1 day after the biopsy), 53 urine samples collected during 4 days to 30 days before the biopsy (36 samples from patients with future No Rejection biopsies and 17 from patients sample from patients with future ACR biopsies), 88 urine samples collected during 31 days to 90 days before the biopsy (68 samples from patients with future No Rejection biopsies and 20 samples from patients sample from patients with future ACR biopsies), and 196 urine samples collected during 91 days to 365 days before the biopsy (139 samples from patients with future No Rejection biopsies and 57 from patients sample from patients with future ACR biopsies) are shown as thin black lines in the one-dimensional scatter plot. The distribution of signature scores are represented by the density shape, and the average for each distribution is shown as a thick black horizontal line crossing the contour of the individual bean plot. The horizontal line across all bean plots (red in the original) indicates the Youden cut-off of the respective signature for the distinguishing ACR biopsies from No Rejection biopsies in biopsy matched urine samples. The Youden cut-off was used to calculate the sensitivity and the specificity of the signature for predicting ACR biopsies at the indicated time intervals. To present comparable datasets, urine samples with valid data for all five signatures in each of the time intervals analyzed were included to generate the bean plots.

FIG. 3A-3E show Bean plots illustrating the specificity of signature in sequential urine samples over time (days post-transplantation) from 40 clinically stable patients. In the parent CTOT-04 study, sequential urine samples were collected on post-transplant days 3, 7, 15 and 30 and in months 2, 3, 4, 5, 6, 9 and 12. FIG. 3A graphically illustrates the log ratio of 3SL/X over time. As illustrated, the cut-off between samples exhibiting acute cellular rejection and samples exhibiting no rejection is about 0.59836. FIG. 3B graphically illustrates the combined metabolite signature (i.e., the combination of log ratios of 3SL/X and quinolinate to X-16397). As illustrated, the cut-off between samples exhibiting acute cellular rejection and samples exhibiting no rejection is about 0.4271. FIG. 3C graphically illustrates the log score of the 18S rRNA normalized measures of CD3 mRNA, IP-10 mRNA and 18S rRNA signature (RNA signature) over time. As illustrated, the cut-off between samples exhibiting acute cellular rejection and samples exhibiting no rejection is about negative 1.563099 (i.e., −1.563099). FIG. 3D graphically illustrates a combination of the log ratio 3 SL/X and the log score of the RNA signature over time. As illustrated, the cut-off between samples exhibiting acute cellular rejection and samples exhibiting no rejection is about negative 0.8815479 (i.e., −0.8815479). FIG. 3E graphically illustrates the composite signature (i.e., a combination of log ratios of 3 SL/X and quinolinate to X-16397 and the log score of the RNA signature) over time. As illustrated, the cut-off between samples exhibiting acute cellular rejection and samples exhibiting no rejection is about negative 0.5095 (i.e., −0.5095). Criteria for classification as patients with stable graft function are listed in the legend to FIG. 1. The metabolite signature and the RNA signature scores from the 56 urine samples collected during the first week of transplantation (0 to 7 days), 29 urine samples collected during week two post-transplant (8 to 14 days), 45 urine samples collected during post-transplant weeks three to four (15 to 30 days), 77 urine samples collected during post-transplant months two and three (31 to 90 days), 103 urine samples collected during months four and six post-transplant (91 to 180 days), 36 urine samples collected during months seven and nine post-transplant (181 to 270 days), and 40 urine samples collected during months ten and twelve post-transplant (271 to 365 days) are shown as thin black lines in the one-dimensional scatter plot. The distribution of signature scores is represented by the density shape, and the average for each distribution is shown as a thick black horizontal line crossing the contour of the individual bean plot. The horizontal line (red in the original) across full breadth of each of the bean plots indicates the Youden cut-off of the respective signature for the distinguishing ACR biopsies from No Rejection biopsies in biopsy matched urine samples and the Youden cut-off was used to calculate specificity of the signatures.

### DETAILED DESCRIPTION

Methods and devices are described herein for detecting, monitoring, and predicting acute cellular rejection in a subject with a kidney transplant from levels of metabolites in the subject's urine. As described herein, noninvasive diagnosis of acute cellular rejection (ACR) of the kidney transplant can accurately be identified and/or predicted by measurement of just four metabolites. These methods and devices can be combined with a diagnostic method that involves quantification of just three RNA transcripts levels. The combination of measurement of just four metabolite levels and of just three RNA transcript levels in urine (estimated cost of the assay: $250 to 300) can reduce biopsy-associated costs and risks.

Described herein is the largest prospective study of metabolite profiling of urine from kidney graft recipients, and the first to investigate the diagnostic accuracy of a composite signature of metabolites and RNAs in urine. A combination of non-targeted LC-MS/MS and GC-MS based metabolomics platforms were used to analyze 1516 urine samples from 241 kidney allograft recipients. Metabolite signatures diagnostic of ACR were identified, including a metabolite signature relating to the ratio of 3-sialyllactose (3SL) to Xanthosine (X) and the ratio of quinolinate to X-16397 was complementary to the information content in the RNA signature.

A composite metabolite-RNA signature was diagnostic of ACR with high accuracy. The composite metabolite and RNA signature was diagnostic of ACR, for examples, in patients who underwent for-cause biopsies and in patients who underwent surveillance biopsies. Applied to urine samples taken four to 30 days collected prior to biopsy, the signatures developed using only biopsy matched urine samples predicted future ACR in pristine samples.

Multivariate machine learning techniques and non-linear fitting algorithms have been applied in biomarker searches. However, metabolites showing a strong association in a linear univariate model are generally the most robust candidate clinical biomarkers. Also, the more metabolites that enter a metabolomics-based signature, the higher are the chances that one of the measurements may fail (e.g., due to ion-suppression in the presence of other metabolites) and thereby invalidate the entire signature. Hence, more sophisticated biomarker discovery algorithms were not employed in the studies described herein.

Each year 15,000 or more kidney transplants are performed in USA alone, and with an estimated incidence of 0.4 biopsies/patient during the first year of transplantation, the charges for performing 7,000 or so biopsies in kidney graft recipients during the first year of transplantation alone can be estimated to be $21 million based the reported charge of $3,000 per biopsy. Collins et al., Am J Kidney Dis 59: A7, e1-420, 2012); Collins et al., Am J Kidney Dis 45: A5-7, S1-280 (2005).

### Metabolites

As described in the Examples, 749 different metabolites from 65 metabolic pathways were detected in 4300 urine samples collected from the 485 kidney graft recipients. Some of these metabolites are highly useful indicators for detection of transplant rejection even before clinically significant rejection occurs.

As described herein for the first time, 3-sialyllactose is linked to kidney allograft rejection. The 3-sialyllactose molecule has CAS number 35890-38-1; and it is also called α-NeuNAc-(2→3)-β-D-Gal-(1→4)-D-Glc, 3′-N-Acetylneuraminyl-D-lactose sodium salt, 3′-SL, 3′-Sialyl-D-lactose, and NANA-Lactose.

A structure for 2,3-sialylactose is shown below.

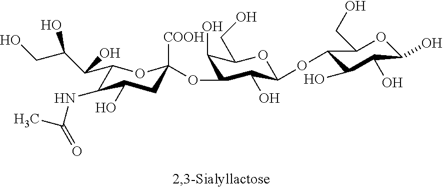

Although the 2,3-sialylactose form of 3-sialylactose is a major form, in some cases, the 3-sialyllactose compound can be linked to other positions of the lactose molecule. For example, the sialyl moiety can be linked via position 2 (as shown above), or via position 4, or via position 5, or via any of positions 7-9 of the sialyl moiety to the lactose molecule.

The 3-sialyllactose has an ion mass of 632.2 grams, and a retention index of 725.6 as detected under basic negative ion optimized conditions using a Waters ACQUITY UPLC and a Thermo-Finnigan LTQ mass spectrometer, having an electrospray ionization (ESI) source and linear ion-trap (LIT) mass analyzer.

Pro-inflammatory (e.g., stimulation of CD11c+ dendritic cells) as well as anti-inflammatory (e.g., inhibition of cholera toxin) properties have been ascribed to this sialylated component. Also, sialyllactose may represent a molecular recognition pattern for dendritic cell capture and contribute to alloantigen presentation and triggering of acute rejection. Their increased levels during ACR may represent aberrant membrane glycolipid metabolism in immune and/or kidney parenchymal cells.

Quinolinate has CAS number 89-00-9; and is also referred to as quinolinic acid, or pyridine-2,3-dicarboxylic acid. It is a dicarboxylic acid with a pyridine backbone, and is a colorless solid. It is the biosynthetic precursor to nicotine. The structure of quniolinate is shown below.

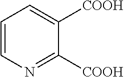 [SMILES: O=C(O)c1cccnc1C(=O)O]

Quinolinate has an ion mass of 296.1, and a retention index of 1697.7, when separating metabolites from urine sample using a Thermo-Finnigan Trace DSQ fast-scanning single-quadruple mass spectrometer using electron impact ionization and operated at unit mass resolving power (GC/MS).

Quinolinate is a product of tryptophan metabolism and is generated from kynurenine via a spontaneous, non-enzymatic reaction, and then oxidized by quinolinate phosphoribosyltransferase to nicotinic acid ribonucleotide, nicotinic acid adenine dinucleotide and nicotinamide adenine dinucleotide (NAD+). By serving as a precursor for the biosynthesis of NAD+, quinolinate may help meet the metabolic demands of activated immune cells contributing to ACR.

The X-16397 compound is readily identified by its ion mass, which is 248.1 g/mole, and its retention index, which is 2200.8 when separating metabolites from urine samples using acidic positive ion optimized conditions (Pos) and detection using a Waters ACQUITY UPLC and a Thermo-Finnigan LTQ mass spectrometer; having an electrospray ionization (ESI) source and linear ion-trap (LIT) mass analyzer (LC/MS Pos).

Xanthosine is also known as 9-[(2R,3R,4S,5R)-3,4-dihydroxy-5-(hydroxymethyl)oxolan-2-yl]-3H-purine-2,6-dione, xanthine riboside, bmse000128, 9-beta-D-ribofuranosylxanthine, beta-D-ribofuranoside, and xanthine-9. The structure of xanthosine is shown below.

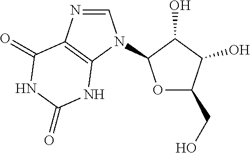 [SMILES: O=c1[nH]c(=O)c2ncn([C@@H]3O[C@H](CO)[C@@H](O)[C@H]3O)c2[nH]1]

Xanthosine has an ion mass of 285 g/mole and a retention index of 1785 when separating metabolites from urine samples using acidic positive ion optimized conditions (Pos) and detection a Waters ACQUITY UPLC and a Thermo-Finnigan LTQ mass spectrometer, having an electrospray ionization (ESI) source and linear ion-trap (LIT) mass analyzer (LC/MS Pos).

Xanthosine is a nucleoside derived from the purine base xanthine and ribose. Xanthine monophosphate (XMP) is produced by inosine monophosphate dehydrogenase (IMPDH) and facilitates proliferation of T and B cells. IMPDH inhibition by mycophenolic acid (MPA) is one mechanism by which MPA reduces the incidence of acute rejection. A potential mechanism for the lower level of xanthosine during ACR is inefficient inhibition of IMPDH leading to efficient conversion of IMP to guanosine and consequent lack of substrate for xanthine biosynthesis.

### Collection of Urine

Urine is collected from transplant recipients. Such transplant recipients can include any recipient of a transplanted organ. For example, in some instances the recipient received an HLA matched transplant organ from a living or deceased donor. Transplant organs can include kidneys, hearts, livers, lungs, pancreas, intestines, and combinations thereof. In some instances, the transplant is a kidney.

Urine is collected from a subject (e.g., fifty to 100 ml of midstream urine) in a sterile, sealed urine collection cup without addition of any preservatives. Urine can be processed within 1 hour of collection. If this was not possible, urine can be refrigerated at 4° C. for a maximum time of 4 hours. The urine sample can be centrifuged to separate urinary cells (that sediment) from metabolites (that do not sediment). For example, urine samples can be centrifuged at 2000 g at room temperature for 30 minutes in sterile disposable tubes. After centrifugation, both the supernatant and the pellet can be processed or stored at −80° C. The supernatant can be tested for metabolites of 3-sialyllactose (3SL), xanthosine (X), quinolinate and X-16397. The pellet can be evaluated for expression levels of 18S ribosomal RNA, CD3ε mRNA and interferon inducible protein-10 mRNA.

It can be useful to collect a series of urine samples, for example, to monitor the status of a transplant. For example, samples can be obtained daily, or every two days, or every three days, or every four days, or every five days, or every six days, or once a week, or twice a week, or three times a week, or every two weeks, or once every month, or once every two months, or at selected times that are convenient for the subject.

Urine samples can be de-salted, for example, using graphite. As illustrated herein, urinary samples can be de-salted and concentrated using a graphite-carbon (type D) mini-SPE cartridge from Agilent Technologies.

Urine samples (e.g., urine supernatants) can be concentrated or diluted in some cases. Because ratios of metabolites in a particular sample are the indicator of transplant rejection, dilution or concentration of a sample does not affect such ratios. For example, urine samples can be diluted in solvents conveniently employed for analysis by various chromatographic or spectroscopic equipment. Examples of solvents or diluents that can be employed include water, acetonitrile, and/or ammonium hydroxide.

### Metabolites Analysis

Urine samples can be evaluated for metabolites using gas chromatography, mass spectrometry, liquid chromatography, high pressure liquid chromatography, or combinations thereof. In some cases, a combination of gas chromatography and mass spectrometry (GC/MS) and/or a combination of liquid chromatography and mass spectrometry (LC/MS or LC/MS/MS) can be employed to determine the amounts of metabolites in samples. For example, in some cases a combination of a gas chromatography (GC) column such as a 5% diphenyl/95% dimethyl polysiloxane fused silica column and a Quadrupole Time-of-Flight (QTOF) mass spectrometer can be employed. For example, in some instances a fast-scanning single-quadruple mass spectrometer using electron impact ionization can be employed. In some cases, a Waters ACQUITY UPLC and a Thermo-Finnigan LTQ mass spectrometer, which consisted of an electrospray ionization (ESI) source and linear ion-trap (LIT) mass analyzer can be employed.

The ratios of 3-sialyllactose/xanthosine or quinolinate/X-16397 identifies transplant rejection in urinary samples of subjects undergoing transplant rejection or in urinary samples of subjects who will develop transplant rejection in about four to thirty days.

Use of ratios of two urine metabolites (e.g., 3-sialyllactose and xanthosine; or quinolinate and X-16397) eliminates the problem of normalization and thus renders these metabolite biomarkers more robust and easily applicable in the clinic. Ratios of metabolite concentrations can be developed efficiently, for example, into a targeted mass spectrometric assay as ratio of two metabolite-specific fragmentation patterns (MRMs), thereby obviating the need for measurement of an external calibration standards, such as urine creatinine or osmolality.

For example, the ratios of 3-sialyllactose/xanthosine (e.g., log₁₀(3-sialyllactose/xanthosine)) and quinolinate/X-16397 (e.g., log₁₀(quinolinate/X-16397)) are detectably different in urine samples of subjects who have ongoing transplant rejection, or who will develop transplant rejection than in urine samples of subjects who are not undergoing or will not develop transplant rejection for at least the next 4 days, or at least the next 10 days, or at least the next 30 days, or for at least 31-90 days. For example, transplant rejection (e.g., acute cellular rejection) can or will be ongoing in subjects where the subjects' urinary samples have log ratios of 3-sialyllactose/xanthosine and quinolinate/X-16397 that are detectably different by about 5%, or about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95%, or about 100%, or about 150% than these log ratios are in samples from subjects that are not undergoing transplant rejection for at least the next 4-30 days, or 31-90 days (e.g., No Rejection biopsy-matched subject samples).

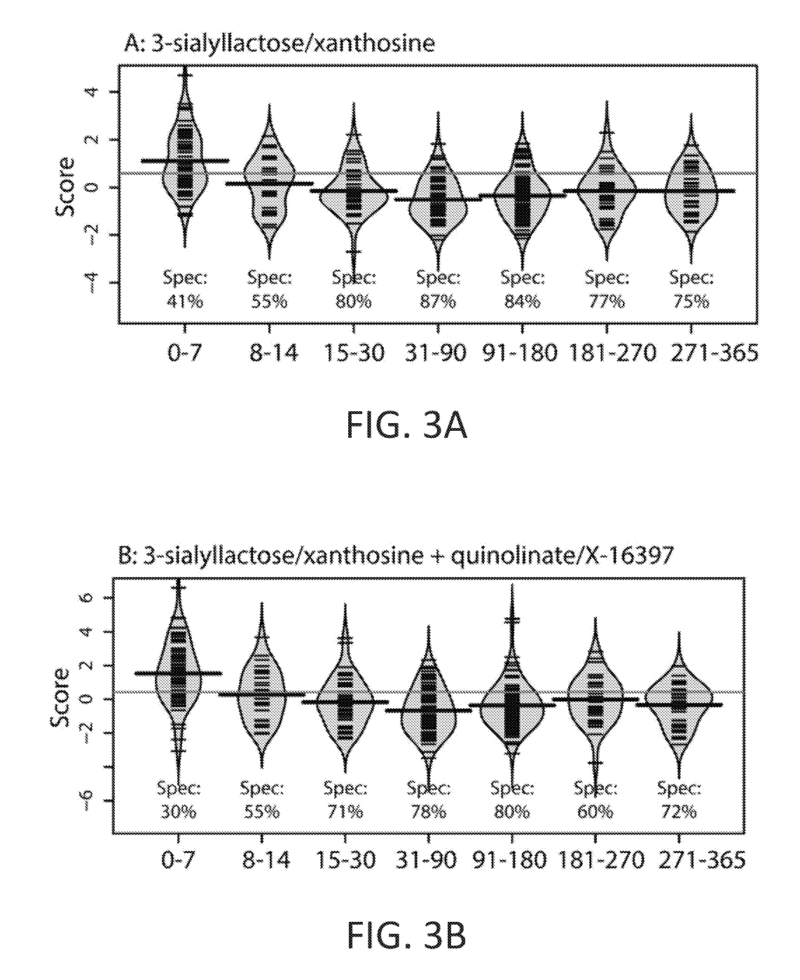

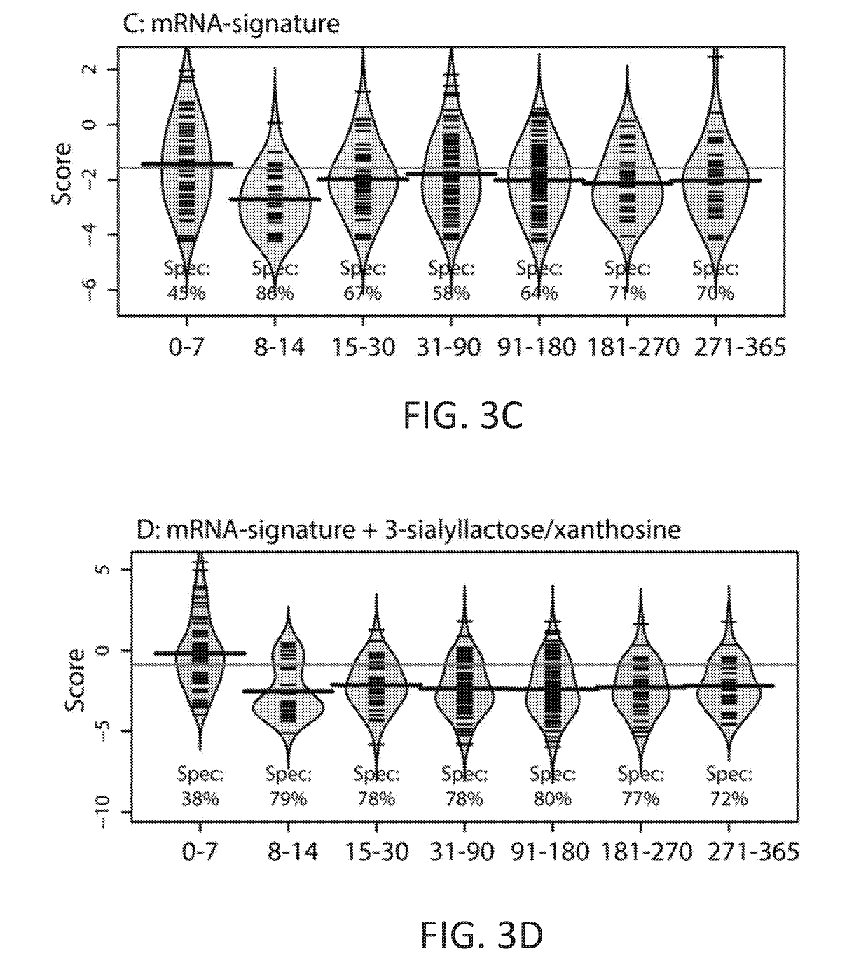

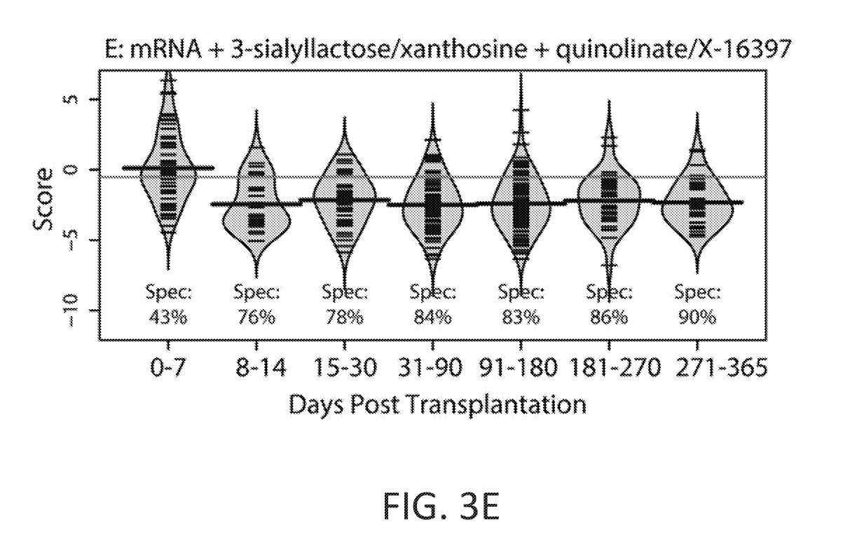

Analysis involving receiver-operating-characteristic (ROC) curves identified a specific cut-off value that identified and predicted acute cellular rejection with a sensitivity of 59% and a specificity of 76% by a ratio of 3-sialyllactose/xanthosine. In some cases, transplant rejection is occurring or will occur when urine sample is tested and identified as having a 3-sialyllactose/xanthosine log ratio higher than 0.5, or higher than 0.52, or higher than 0.55, or higher than 0.56, or higher than 0.58, or higher than 0.59, or higher than 0.59836, than these ratios are in samples from subjects that are not undergoing transplant rejection (e.g., No Rejection biopsy-matched subject samples from sample not undergoing transplant rejection for the next 4-30 days, or next 31-90 days). See, e.g., FIG. 3A-3E (especially FIG. 3A).

In some cases, the cut-off is 0.59836 for a log ratio of mean levels of 3-sialyllactose/xanthosine metabolites in urine samples, where subjects have or will have acute cellular rejection if their urine sample has a sialyllactose/xanthosine log ratio higher than 0.59836.

Analysis involving ROC curve also identified a cut-off value that identified or predicted acute cellular rejection with a sensitivity of 82% and a specificity of 71% when anyalyzing the combination of ratios of 3-sialyllactose/xanthosine and quinolinate/X-16397 metabolite levels using. Hence a combined metabolite diagnostic signature of metabolite levels was an excellent indicator of whether or not a subject is exhibiting symptoms of transplant rejection or will exhibit symptoms of transplant rejection. For example, the following combined metabolite signature can be employed to diagnose transplant rejection.

Log₁₀(3-sialyllactose/xanthosine)+0.9513*log₁₀(quinolinate/X-16397).

In some cases, transplant rejection is occurring or will occur when urine sample is tested and identified as having a combined metabolite signature higher than 0.2, or higher than 0.3, or higher than 0.35, or higher than 0.37, or higher than 0.38, or higher than 0.39, or higher than 0.4, or higher than 0.41, or higher than 0.42, or higher than 0.4271. Such a cut-off distinguishes subjects currently exhibiting symptoms of transplant rejection and subjects who will exhibit symptoms of transplant rejection in the next 4-30 days, or next 31-90 days from subjects that are not undergoing transplant rejection. See, e.g., FIG. 3A-3E (especially FIG. 3B).

In some cases, a reliable cut-off of such a combined metabolite signature in subjects that are or will undergo acute cellular rejection and a “No Rejection” group of subjects is 0.4271. For example, transplant rejection (e.g., acute cellular rejection) can or will be ongoing in subjects where the subjects' urinary samples have a combined metabolite signature that is above 0.4271.

The combined metabolite signature can also predict that transplant rejection will occur in subjects who do not currently have detectable transplant rejection symptoms. For example, the combined metabolite signature is detectably different in urine samples of subjects who are ongoing or will undergo transplant rejection than in urine samples of subjects who are not undergoing or will not undergo transplant rejection for at least the next 4-30 days, or for at least the next 31-90 days.

In some embodiments, the combined metabolite signature can be greater than about 0.4, or greater than 0.41, or greater than 0.42, or greater than 0.4271, or greater than 0.45 when transplant rejection is occurring or will occur.

The diagnostic signatures for such metabolite ratios were remarkably stable in sequential urine samples collected from clinically stable patients. Hence, these signatures can help reduce the need for surveillance biopsies in this patient population. The determination described herein that such metabolite signatures cross the diagnostic threshold during the month prior to an ACR biopsy shows that these signatures can help initiate preemptive anti-rejection therapy and treatment prior to changes in renal allograft function.

### RNA Signature as an Indicator of Transplant Rejection.

As described in Applicants' U.S. Ser. No. 14/170,132, which is specifically incorporated herein by reference in its entirety, acute cellular rejection can be noninvasively and accurately diagnosed using a 3-gene signature determined from quantified levels of CD3ε mRNA, IP-10 mRNA and 18S rRNA in urine samples.

For example, the 3-gene RNA diagnostic signature measured in urine specimens from clinically stable allograft patients can be used to monitor the likelihood of such a patient to subsequently develop acute cellular rejection. The studies reported in U.S. Ser. No. 14/170,132 demonstrate that the diagnostic signature obtained from measurements conducted on urine specimens from patients with normal allograft biopsies and in clinically stable patients was relatively flat and distinct compared to the progressive increase observed in specimens from those who later developed biopsy confirmed acute cellular rejection.

The 3-gene RNA diagnostic signature can also serve to direct the immunosuppressive therapy of a transplant patient. The levels of this signature reflect the potency of immunosuppressive regimens. For example, a marked rise in the RNA levels in the absence of clinical manifestations of acute cellular rejection indicates that preemptive anti-rejection therapy can be needed.

In addition, the gene signature can distinguish acute cellular rejection from antibody-mediated rejection (AMR), borderline and other changes.

The RNA expression levels correlated with development of acute cellular rejection (and other kidney problems) include 18S-normalized CD3ε mRNA, IP-10 mRNA and 18S rRNA expression levels. In some cases, a diagnostic signature of RNA expression levels can be employed to identify transplant rejection.

RNA signature=−6.1487+0.8534 log₁₀(CD3ε/18S)+0.6376 log₁₀(IP-10/18S)+1.6464 log₁₀(18S)
where:
   CD3ε is the absolute urinary CD3ε mRNA copy number per microgram of total RNA in the urine sample;
   IP-10 is the absolute urinary IP-10 mRNA copy number per microgram of total RNA in the urine sample; and
   18S is the an absolute urinary 18S rRNA copy number per microgram of total RNA in the urine sample times 10⁻⁶;
to thereby detect a developing or existing dysfunction or rejection of a kidney transplant in the subject.

The 3-gene RNA signature discriminated acute cellular rejection biopsies from biopsies without rejection. For example, the AUC was 0.85 (95% CI: 0.78-0.91, P<0.0001) by ROC curve analysis. The optimism-adjusted AUC was 0.83 by bootstrap re-sampling and the Hosmer-Lemeshow test indicated excellent fit (P=0.77). The calibration curve showed excellent calibration (Cox's intercept=−0.06; slope=0.92). In an external validation dataset, the AUC was 0.74 (95% CI: 0.61 to 0.86, P=0.0002) and not different from CTOT-04's AUC (P=0.13). The RNA signature distinguished ACR from acute antibody mediated rejection and borderline rejection (AUC=0.78, P<0.0001), patients induced with anti-IL-2 receptor antibodies from T cell depleting antibodies (P=0.0009), and diagnostic of ACR in both groups. Urinary tract infection did not impact the RNA signature (P=0.69). The average trajectory of the RNA signature in repeat urine samples remained below the threshold diagnostic of ACR in the group without an ACR whereas there was a sharp rise during the weeks prior to an ACR biopsy (P<0.0001).

Heretofore, robust yardsticks for defining the immune status of the transplant recipient have not been developed. The relatively flat trajectory of the 3-gene diagnostic RNA signature of those who do not manifest acute cellular rejection is in contrast to this signature's increasing trajectory in those who do develop acute cellular rejection. Thus, the 3-gene RNA diagnostic signature provides a tool not only for detecting acute cellular rejection but also for monitoring a subject's immune status and for titrating an appropriate immunosuppressive therapy. The finding that the RNA signature can reflect the potency of immunosuppressive therapy offers new opportunities for more precise treatment and reduced trauma to the patient.

Although acute cellular rejection is frequently treatable, it is a well-recognized precursor of chronic rejection and ultimate graft loss. Current preventive strategies include immunosuppression, initiated at the time of transplant with adjustments in medications made based on drug levels, toxicity and clinical events such as increased creatinine. The marked increase in the trajectory of the diagnostic RNA signature in 1-12 weeks preceding acute cellular rejection, in addition to foreshadowing the development of ACR, offers new opportunities for preemptive anti-rejection therapy, prior to irreversible tissue injury.

In sum, the well-calibrated, parsimonious diagnostic RNA signature described herein is determined from the RNA expression levels of three genes relevant to acute cellular rejection. The RNA signature provides both physician and patient with direct measures of risk (the predicted probability that a biopsy would reveal acute cellular rejection) and a means of assessing progress/decline over repeated assessments. The RNA signature provides a reliable method for discrimination and diagnosis and an exceptional tool for assessing the likelihood of acute cellular rejection in a given patient at any point following transplantation. The results of the CTOT-4 clinical trial study described herein show that, in addition to minimizing invasive biopsies, the urinary cell mRNA and rRNA profiling described here can direct preemptive anti-rejection therapy and personalized immunosuppression.

Diagnostic signature algorithms are provided herein that can be employed in a method for detecting, monitoring and diagnosing kidney function from a urine sample obtained from a subject.

A method for detecting developing or existing dysfunction or rejection of a kidney transplant in a subject from a urine sample obtained from the subject that includes consideration of RNA expression levels can involve:
(a) determining an absolute urinary CD3ε mRNA, and absolute urinary IP-10 mRNA copy numbers per microgram of total RNA in the urine sample;
(b) determining an absolute urinary 18S rRNA copy number per microgram of total RNA times 10⁻⁶ in the urine sample;
(c) ascertaining a diagnostic signature of developing or existing dysfunction or rejection of a kidney transplant in the subject with the following algorithm:

RNA signature=−6.1487+0.8534 log₁₀(CD3ε/18S)+0.6376 log₁₀(IP-10/18S)+1.6464 log₁₀(18S)

where:
CD3ε is the absolute urinary CD3ε mRNA copy number per microgram of total RNA in the urine sample;
IP-10 is the absolute urinary IP-10 mRNA copy number per microgram of total RNA in the urine sample; and
18S is the an absolute urinary 18S rRNA copy number per microgram of total RNA in the urine sample times 10⁻⁶;
to thereby detect a developing or existing dysfunction or rejection of a kidney transplant in the subject.

The RNA expression levels are determined as described herein, or using other procedures available to those of skill in the art.

An RNA diagnostic signature above −0.7, or above −0.8, or above −0.9, or above −1.0, or above −1.1, or above −1.2, or above −1.3, or above −1.4, or above −1.5, or above −1.6, or above −1.7, indicates that the subject can be undergoing transplant rejection or can develop transplant rejection in the next 8 days to 365 days. See FIG. 3C. For example, in some cases when the value of the RNA diagnostic signature is below a threshold of −1.563099 (or below −1.56) the subject is not prone to development of tissue rejection. However, when testing of a subject's urine sample yields an RNA signature above a threshold of −1.563099 (or above −1.56) treatment of the subject for tissue rejection is indicated.

Acute cellular rejection was defined as Banff Grade 1A or higher and No Rejection biopsies were those classified by the on-site pathologist as showing no histological features of rejection. From those models in which each predictor was significant at P<0.05, one with the greatest log-likelihood and greatest area under the receiver-operating-characteristic (ROC) curve was provisionally selected as the best-fitting model. The regression estimates from this model defined a diagnostic signature, and area under the curve (AUC), sensitivity, and specificity were used to evaluate the ability of this signature to discriminate ACR biopsies from No Rejection biopsies.

The model was validated in several ways. The generalizability of the fitted model to other data sets was evaluated using bootstrap re-sampling methods. Logistic regression with backwards elimination was used to identify the best subset of the RNA measures and 18S rRNA in each of 500 data sets obtained by sampling with replacement from the original data set. The best subset model was then fit to 500 additional bootstrap samples from which optimism-adjusted measures of discrimination (i.e., AUC) and model fit (i.e., Cox's intercept and slope) and a LOESS-smoothed calibration plot, were obtained (LOESS: locally estimated scatterplot smoothing).

ROC curve analysis showed that this 3-gene signature yielded an AUC of 0.85 (95% confidence interval [CI] 0.78 to 0.91, P<0.0001). Using the cut-off point of −1.563099 (or −1.56), which maximizes Youden's index, this diagnostic signature has 79% sensitivity and 78% specificity to discriminate ACR biopsies from No Rejection biopsies (see FIG. 3C; and U.S. Ser. No. 14/170,132, FIG. 3A).

The Hosmer-Lemeshow test indicated an excellent fit of this model to the data (chi-square χ²=4.84, with 8 df, and P=0.77). The 3-gene signature also discriminated between the group of patients with biopsy specimens showing acute cellular rejection and the group of patients with stable graft function who did not undergo biopsy ((see U.S. Ser. No. 14/170,132, FIG. 3B).

Bootstrap validation of this three-gene model yielded a cross-validated estimate of the AUC of 0.83, which is an estimate of the expected value of the AUC in independent samples (i.e., samples not used to derive the diagnostic signature). The calibration-curve intercept and slope of −0.06 and 0.92, respectively, revealed that the predicted probabilities of a biopsy showing acute cellular rejection, across the range of the diagnostic signature, tended to be only very slightly higher than the actual probabilities (see U.S. Ser. No. 14/170,132, FIG. 3C) and that the likelihood that the model was over-fitted was small. The loess-smoothed estimates of the unadjusted and cross-validated calibration curves were overlaid on a diagonal reference line representing perfect model calibration ((see U.S. Ser. No. 14/170,132, FIG. 3C). The close correspondence of the two curves to the reference line shows excellent fit and reflects the above interpretation of the intercept and slope estimates of the calibration curve.

The RNA signature can be combined with any of the metabolite signatures described herein. For example, the following combined signatures can be employed to discriminate kidney transplant subjects who are or will undergo transplant rejection from those who will not.

RNA signature+1.1164*log(3-sialyllactose/xanthosine)

RNA signature+0.8932*log(quinolinate/X-16397)

For example, when a 3-sialyllactose/xanthosine+RNA signature is used, a cut-off value that distinguishes subjects that can be undergoing transplant rejection or can develop transplant rejection, from subjects who are not and/or will not under transplant rejection can be about −0.88, or about −0.8815479. Thus, in some cases, patients undergoing transplant rejection or that can develop transplant rejection have a 3-sialyllactose/xanthosine+RNA signature above −0.87, or above −0.88, or above −0.8815479, or above −0.89, or above −0.9, or above −0.91 (where “above” means less negative).

A composite of the 3-sialyllactose/xanthosine, quinolinate/X-16397, and RNA signatures called a composite metabolite-RNA diagnostic signature has the following formula:

RNA-signature+1.1164*log(3-sialyllactose/xanthosine)+0.6937*log(quinolinate/X-16397).

FIG. 3A-3E show cut-off values for various diagnostic signatures. For example, samples with a composite metabolite-RNA diagnostic signature above 0, or above −0.1, or above −0.2, or above −0.3, or above −0.4, or above −0.5, or above −0.6 (where “above” means less negative), indicates that the subject can be undergoing transplant rejection or can develop transplant rejection in the next 8 days to 365 days. In some cases, −0.5095 is a reliable cut-off value that distinguishes subjects that can be undergoing transplant rejection or can develop transplant rejection from subjects who are not and/or will not under transplant rejection. See FIG. 3E.

Determination of CDR mRNA, IP-10 mRNA and 18S rRNA Expression Levels

Any procedure available to those of skill in the art can be employed to determine the expression levels of CD3ε mRNA, IP-10 mRNA and 18S rRNA. For example, probes, primers, and/or antibodies can be employed in quantitative nucleic acid amplification reactions (e.g., quantitative polymerase chain reaction (PCR)), primer extension, Northern blot, immunoassay, immunosorbent assay (ELISA), radioimmunoassay (RIA), immunofluorimetry, immunoprecipitation, equilibrium dialysis, immunodiffusion, immunoblotting, mass spectrometry and other techniques available to the skilled artisan.

In some embodiments, the expression levels of CD3ε mRNA, IP-10 mRNA and 18S rRNA are determined using probes or primers that can hybridize to the CD3ε mRNA, IP-10 mRNA or 18S rRNA. Sequences for CD3ε mRNA, IP-10 mRNA and 18S rRNA are readily available and can be used to make such probes and primers.

For example, the following sequence for a human 18S rRNA is available from the National Center for Biotechnology Information database (see website at ncbi.nlm.nih.gov) as accession number K03432 (SEQ ID NO:1).

| | |
|---|---|
| 1 | CGCTGCTCCT CCCGTCGCCG TCCGGGCCCG TCCGTCCGTC |
| 41 | CGTCCGTCGT CCTCCTCGCT NNNNCGGGGC GCCGGGCCCG |
| 61 | TCCTCACNGG CCCCCGNNNN NGTCCNGGCC CGTCGGGGCC |
| 121 | TCGCCGCGCT CTACCTTACC TACCTGGTTG ATCCTGCCAG |
| 161 | TAGCATATGC TTGTCTCAAA GATTAAGCCA TGCATGTCTA |
| 201 | AGTACGCACG GCCGGTACAG TGAAACTGCG AATGGCTCAT |
| 241 | TAAATCAGTT ATGGTTCCTT TGGTCGCTCG CTCCTCTCCT |
| 281 | ACTTGGATAA CTGTGGTAAT TCTAGAGCTA ATACATGCCG |
| 321 | ACGGGCGCTG ACCCCCTTCG CGGGGGGGAT GCGTGCATTT |
| 361 | ATCAGATCAA AACCAACCCG GTCAGCCCCT CTCCGGCCCC |
| 401 | GGCCGGGGGG CGGGCGCCGG CGGCTTTGGT GACTCTAGAT |
| 441 | AACCTCGGGC CGATCGCACG CCCCCCGTGG CGGCGACGAC |
| 481 | CCATTCGAAC GTCTGCCCTA TCAACTTTCG ATGGTAGTCG |
| 521 | CCGTGCCTAC CATGGTGACC ACGGGTGACG GGGAATCAGG |
| 561 | GTTCGATTCC GGAGAGGGAG CCTGAGAAAC GGCTACCACA |
| 601 | TCCAAGGAAG GCAGCAGGCG CGCAAATTAC CCACTCCCGA |
| 641 | CCCGGGGAGG TAGTGACGAA AAATAACAAT ACAGGACTCT |
| 681 | TTCGAGGCCC TGTAATTGGA ATGAGTCCAC TTTAAATCCT |
| 721 | TTAACGAGGA TCCATTGGAG GGCAAGTCTG GTGCCAGCAG |
| 761 | CCGCGGTAAT TCCAGCTCCA ATAGCGTATA TTAAAGTTGC |
| 801 | TGCAGTTAAA AAGCTCGTAG TTGGATCTTG GGAGCGGGCG |
| 841 | GGCGGTCCGC CGCGAGGCGA GCCACCGCCC GTCCCCGCCC |
| 881 | CTTGCCTCTC GGCGCCCCCT CGATGCTCTT AGCTGAGTGT |
| 921 | CCCGCGGGGC CCGAAGCGTT TACTTTGAAA AAATTAGAGT |
| 961 | GTTCAAAGCA GGCCCGAGCC GCCTGGATAC CGCAGCTAGG |
| 1001 | AATAATGGAA TAGGACCGCG GTTCTATTTT GTTGGTTTTC |
| 1041 | GGAACTGAGG CCATGATTAA GAGGGACGGC CGGGGGCATT |
| 1081 | CGTATTGCGC CGCTAGAGGT GAAATTCCTT GGACCGGCGC |
| 1121 | AAGACGGACC AGAGCGAAAG CATTTGCCAA GAATGTTTTC |
| 1161 | ATTAATCAAG AACGAAAGTC GGAGGTTCGA AGACGATCAG |
| 1201 | ATACCGTCGT AGTTCCGACC ATAAACGATG CCGACCGGCG |
| 1241 | ATGCGGCGGC GTTATTCCCA TGACCCGCCG GGCAGCTTCC |
| 1281 | GGGAAACCAA AGTCTTTGGG TTCCGGGGGG AGTATGGTTG |
| 1321 | CAAAGCTGAA ACTTAAAGGA ATTGACGGAA GGGCACCACC |
| 1361 | AGGAGTGGAG CCTGCGGCTT AATTTGACTC AACACGGGAA |
| 1401 | ACCTCACCCG GCCCGGACAC GGACAGGATT GACAGATTGA |
| 1441 | TAGCTCTTTC TCGATTCCGT GGGTGGTGGT GCATGGCCGT |
| 1481 | TCTTAGTTGG TGGAGCGATT TGTCTGGTTA ATTCCGATAA |
| 1521 | CGAACGAGAC TCTGGCATGC TAACTAGTTA CGCGACCCCC |
| 1561 | GAGCGGTCGG CGTCCCCCAA CTTCTTAGAG GGACAAGTGG |
| 1601 | CGTTCAGCCA CCCGAGATTG AGCAATAACA GGTCTGTGAT |
| 1641 | GCCCTTAGAT GTCCGGGGCT GCACGCGCGC TACACTGACT |
| 1681 | GGCTCAGCGT GTGCCTACCC TACGCCGGCA GGCGCGGGTA |
| 1721 | ACCCGTTGAA CCCCATTCGT GATGGGGATC GGGGATTGCA |
| 1761 | ATTATTCCCC ATGAACGAGG AATTCCCAGT AAGTGCGGGT |
| 1801 | CATAAGCTTG CGTTGATTAA GTCCCTGCCC TTTGTACACA |
| 1841 | CCGCCCGTCG CTACTACCGA TTGGATGGTT TAGTGAGGCC |
| 1881 | CTCGGATCGG CCCCGCCGGG GTCGGCCCAC GGCCCTGGCG |
| 1921 | GAGCGCTGAG AAGACGGTCG AACTTGACTA TCTAGAGGAA |
| 1961 | GTAAAAGTCG TAACAAGGTT TCCGTAGGTG AACCTGCGGA |
| 2001 | AGGATCATTA ACGGAGCCCG GACGGCGGCC CGCGGCGGCG |
| 2041 | CCGCGCCGCG CTTCCCTCCG CACACCCACC CCCCCACCGC |
| 2081 | GACGGCGCGT GCGGGCGGGG CCGTGCCCGT TCGTTCGCTC |
| 2121 | GCTCGTTCGT TCGCCGCCCG GCCCGGCCGC GAGAGCCGAG |
| 2161 | AACTCGGGAG GGAGACGGGG GAGAGAGAGA GAGAGAGAGA |
| 2201 | GAGAGAGAGA GAGAGAGAGA GAAAGAAGGG CGTGT |

A cDNA sequence for a human CD3ε is also available from the National Center for Biotechnology Information database as accession number NM_000733 (SEQ ID NO:2.

| | |
|---|---|
| 1 | TATTGTCAGA GTCCTCTTGT TTGGCCTTCT AGGAAGGCTG |
| 41 | TGGGACCCAG CTTTCTTCAA CCAGTCCAGG TGGAGGCCTC |
| 81 | TGCCTTGAAC GTTTCCAAGT GAGGTAAAAC CCGCAGGCCC |
| 121 | AGAGGCCTCT CTACTTCCTG TGTGGGGTTC AGAAACCCTC |
| 161 | CTCCCCTCCC AGCCTCAGGT GCCTGCTTCA GAAAATGAAG |
| 201 | TAGTAAGTCT GCTGGCCTCC GCCATCTTAG TAAAGTAACA |
| 241 | GTCCCATGAA ACAAAGATGC AGTCGGGCAC TCACTGGAGA |
| 281 | GTTCTGGGCC TCTGCCTCTT ATCAGTTGGC GTTTGGGGGC |
| 321 | AAGATGGTAA TGAAGAAATG GGTGGTATTA CACAGACACC |
| 361 | ATATAAAGTC TCCATCTCTG GAACCACAGT AATATTGACA |
| 401 | TGCCCTCAGT ATCCTGGATC TGAAATACTA TGGCAACACA |
| 441 | ATGATAAAAA CATAGGCGGT GATGAGGATG ATAAAAACAT |
| 481 | AGGCAGTGAT GAGGATCACC TGTCACTGAA GGAATTTTCA |
| 521 | GAATTGGAGC AAAGTGGTTA TTATGTCTGC TACCCCAGAG |
| 561 | GAAGCAAACC AGAAGATGCG AACTTTTATC TCTACCTGAG |
| 601 | GGCAAGAGTG TGTGAGAACT GCATGGAGAT GGATGTGATG |
| 641 | TCGGTGGCCA CAATTGTCAT AGTGGACATC TGCATCACTG |
| 681 | GGGGCTTGCT GCTGCTGGTT TACTACTGGA GCAAGAATAG |
| 721 | AAAGGCCAAG GCCAAGCCTG TGACACGAGG AGCGGGTGCT |
| 761 | GGCGGCAGGC AAAGGGGACA AAACAAGGAG AGGCCACCAC |
| 801 | CTGTTCCCAA CCCAGACTAT GAGCCCATCC GGAAAGGCCA |
| 841 | GCGGGACCTG TATTCTGGCC TGAATCAGAG ACGCATCTGA |
| 881 | CCCTCTGGAG AACACTGCCT CCCGCTGGCC CAGGTCTCCT |
| 921 | CTCCAGTCCC CCTGCGACTC CCTGTTTCCT GGGCTAGTCT |
| 961 | TGGACCCCAC GAGAGAGAAT CGTTCCTCAG CCTCATGGTG |
| 1001 | AACTCGCGCC CTCCAGCCTG ATCCCCCGCT CCCTCCTCCC |
| 1041 | TGCCTTCTCT GCTGGTACCC AGTCCTAAAA TATTGCTGCT |
| 1081 | TCCTCTTCCT TTGAAGCATC ATCAGTAGTC ACACCCTCAC |
| 1121 | AGCTGGCCTG CCCTCTTGCC AGGATATTTA TTTGTGCTAT |
| 1161 | TCACTCCCTT CCCTTTGGAT GTAACTTCTC CGTTCAGTTC |
| 1201 | CCTCCTTTTC TTGCATGTAA GTTGTCCCCC ATCCCAAAGT |
| 1241 | ATTCCATCTA CTTTTCTATC GCCGTCCCCT TTTGCAGCCC |
| 1281 | TCTCTGGGGA TGGACTGGGT AAATGTTGAC AGAGGCCCTG |
| 1321 | CCCCGTTCAC AGATCCTGGC CCTGAGCCAG CCCTGTGCTC |
| 1361 | CTCCCTCCCC CAACACTCCC TACCAACCCC CTAATCCCCT |
| 1401 | ACTCCCTCCA CCCCCCCTCC ACTGTAGGCC ACTGGATGGT |
| 1441 | CATTTGCATC TCCGTAAATG TGCTCTGCTC CTCAGCTGAG |
| 1481 | AGAGAAAAAA ATAAACTGTA TTTGGCTGCA AGAAAAAAAA |
| 1521 | AAAAAAAAAA AAAA |

The following cDNA sequence is also available for a human IP-10 from the National Center for Biotechnology Information database as accession number NM_001565.1 (GI:4504700) (SEQ ID NO:3).

| | |
|---|---|
| 1 | GAGACATTCC TCAATTGCTT AGACATATTC TGAGCCTACA |
| 41 | GCAGAGGAAC CTCCAGTCTC AGCACCATGA ATCAAACTGC |
| 81 | GATTCTGATT TGCTGCCTTA TCTTTCTGAC TCTAAGTGGC |
| 121 | ATTCAAGGAG TACCTCTCTC TAGAACCGTA CGCTGTACCT |
| 161 | GCATCAGCAT TAGTAATCAA CCTGTTAATC CAAGGTCTTT |
| 201 | AGAAAAACTT GAAATTATTC CTGCAAGCCA ATTTTGTCCA |
| 241 | CGTGTTGAGA TCATTGCTAC AATGAAAAAG AAGGGTGAGA |
| 281 | AGAGATGTCT GAATCCAGAA TCGAAGGCCA TCAAGAATTT |
| 321 | ACTGAAAGCA GTTAGCAAGG AAATGTCTAA AAGATCTCCT |
| 361 | TAAAACCAGA GGGGAGCAAA ATCGATGCAG TGCTTCCAAG |
| 401 | GATGGACCAC ACAGAGGCTG CCTCTCCCAT CACTTCCCTA |
| 441 | CATGGAGTAT ATGTCAAGCC ATAATTGTTC TTAGTTTGCA |
| 481 | GTTACACTAA AAGGTGACCA ATGATGGTCA CCAAATCAGC |
| 521 | TGCTACTACT CCTGTAGGAA GGTTAATGTT CATCATCCTA |
| 561 | AGCTATTCAG TAATAACTCT ACCCTGGCAC TATAATGTAA |
| 601 | GCTCTACTGA GGTGCTATGT TCTTAGTGGA TGTTCTGACC |
| 641 | CTGCTTCAAA TATTTCCCTC ACCTTTCCCA TCTTCCAAGG |
| 681 | GTACTAAGGA ATCTTTCTGC TTTGGGGTTT ATCAGAATTC |
| 721 | TCAGAATCTC AAATAACTAA AAGGTATGCA ATCAAATCTG |
| 761 | CTTTTTAAAG AATGCTCTTT ACTTCATGGA CTTCCACTGC |
| 801 | CATCCTCCCA AGGGGCCCAA ATTCTTTCAG TGGCTACCTA |
| 841 | CATACAATTC CAAACACATA CAGGAAGGTA GAAATATCTG |
| 881 | AAAATGTATG TGTAAGTATT CTTATTTAAT GAAAGACTGT |
| 921 | ACAAAGTATA AGTCTTAGAT GTATATATTT CCTATATTGT |
| 961 | TTTCAGTGTA CATGGAATAA CATGTAATTA AGTACTATGT |
| 1001 | ATCAATGAGT AACAGGAAAA TTTTAAAAAT ACAGATAGAT |
| 1041 | ATATGCTCTG CATGTTACAT AAGATAAATG TGCTGAATGG |
| 1081 | TTTTCAAATA AAAATGAGGT ACTCTCCTGG AAATATTAAG |
| 1121 | AAAGACTATC TAAATGTTGA AAGATCAAAA GGTTAATAAA |
| 1161 | GTAATTATAA CT |

The level of expression is determined for one or more genes in sample obtained from a subject. The sample can be a fluid sample such as a blood sample, a peripheral blood mononuclear cell (PBMC) sample, a urine sample, a sample of broncho-alveolar lavage fluid, a sample of bile, pleural fluid or peritoneal fluid, any other fluid secreted or excreted by a normally or abnormally functioning allograft, or any other fluid resulting from exudation or transudation through an allograft or in anatomic proximity to an allograft, or any fluid in fluid communication with the allograft. One convenient example of a sample for determination of the level of gene expression is a urine sample.

RNA can be isolated from the samples by procedures available in the art. Commercially available kits can be employed for such isolation. Alternatively, the urine sample can be treated to lyse any cells therein and the RNA expression levels can be determined with little or no RNA purification step.

For example, the CD3ε mRNA, IP-10 mRNA and 18S rRNA can be determined from a urinary cell sample from the recipient of an organ transplant. Any method known to those in the art can be employed for determining the level of CD3ε mRNA, IP-10 mRNA and 18S rRNA. For example, total RNA, which includes mRNA and rRNA, can be isolated from the sample by use of a commercial kit, such as the TRI Reagent® commercially available from Molecular Research Center, Inc. (Cincinnati, Ohio), can be used to isolate RNA.

Any method available to those in the art can be employed for determining the level of gene expression. Examples include Microarrays, TaqMan® Gene Expression Assays (Applied Biosystems), TaqMan® Low Density Array microfluidic cards (Applied Biosystems), molecular beacons, scorpions, SYBR Green, and/or RT-PCR. Such methods provide quantitative measurements of RNA levels. The Examples section describes quantitative data obtained by quantitative nucleic acid amplification (e.g., real-time RT-PCR) in a small reaction volume.

In another example, a microarray can be used. Microarrays are known in the art and consist of a surface to which probes that correspond in sequence to gene products (e.g. RNAs, polypeptides, fragments thereof etc.) can be specifically hybridized or bound to a known position. Hybridization intensity data detected by the scanner are automatically acquired and processed by the Affymetrix Microarray Suite (MAS5) software. Raw data is normalized to expression levels using a target intensity of 150.

The transcriptional state of a cell may be measured by other gene expression technologies known in the art. Several such technologies produce pools of restriction fragments of limited complexity for electrophoretic analysis, such as methods combining double restriction enzyme digestion with phasing primers (e.g. EP-Al-0534858), or methods selecting restriction fragments with sites closest to a defined RNA end (e.g. Prashar et al; Proc. Nat. Acad. Sci., 93, 659-663, 1996). Other methods statistically sample cDNA pools, such as by sequencing sufficient bases (e.g. 20-50 bases) in each multiple cDNAs to identify each cDNA, or by sequencing short tags (e.g. 9-10 bases) which are generated at known positions relative to a defined RNA end (e.g. Velculescu, Science, 270, 484-487, 1995) pathway pattern.

The quantification of CD3ε nRNA, IP-10 mRNA and 18S rRNA from the total RNA of a sample can be performed by any method known to those in the art. For example, kinetic, quantitative PCR involves reverse transcribing CD3ε mRNA, IP-10 mRNA and 18S rRNA by using reverse-transcriptase polymerase chain reaction (RT-PCR) to obtain CD3ε, IP-10, and 18S rRNA cDNA. The cDNA can then, for example, be amplified by PCR followed by quantitation using a suitable detection apparatus. Determination of CD3ε mRNA, IP-10 mRNA and 18S rRNA expression levels can involve a preamplification step followed by an amplification process. See Example 2 for exemplary methods for quantitation of CD3ε mRNA, IP-10 mRNA and 18S rRNA by kinetic, quantitative PCR.

Amplification systems utilizing, for example, PCR or RT-PCR methodologies are available to those skilled in the art. For a general overview of amplification technology, see, for example, Dieffenbach et al., PCR Primer: A Laboratory Manual, Cold Spring Harbor Laboratory Press, New York (1995).

An alternative method for determining the level of CD3ε mRNA, IP-10 mRNA and 18S rRNA includes the use of molecular beacons and other labeled probes useful in, for example multiplex PCR. In a multiplex PCR assay, the PCR mixture contains primers and probes directed to the CD3ε mRNA, IP-10 mRNA and 18S rRNA. Typically, a single fluorophore is used in the assay. The molecular beacon or probe is detected to determine the level of CD3ε mRNA, IP-10 mRNA and 18S rRNA. Molecular beacons are described, for example, by Tyagi and Kramer (Nature Biotechnology 14, 303-308, (1996)) and by Andrus and Nichols in U.S. Patent Application Publication No. 20040053284.

Another method includes, for instance, quantifying cDNA (obtained by reverse transcribing the CD3ε mRNA, IP-10 mRNA and 18S rRNA using a fluorescence based real-time detection method, such as the ABI PRISM 7500, 7700, or 7900 Sequence Detection System (TaqMan®) commercially available from Applied Biosystems, Foster City, Calif., or similar system as described by Heid et al., (Genome Res. 1996; 6:986-994) and Gibson et al. (Genome Res. 1996; 6:995-1001).

Ribosomal RNA constitutes about 80-85% of the total cellular RNA and is more stable compared to mRNA. The inventors used 5×10⁷ 18 S ribosomal RNA copies per microgram of total RNA as an additional cutoff point to qualify a urine specimen. A threshold of 5×10⁷ copies of rRNA ensured that 25,000 rRNA copies (rRNA from about 10 cells in view of data showing that 2501 copies of 18S rRNA are typically present in a single human peripheral blood mononuclear cell) were present in the 2.5 ul cDNA used in 1:2000 dilutions for measuring 18S rRNA abundance in the PCR assay.

In some embodiments, the expression level is determined using log-transformed levels of CD3ε mRNA, IP-10 mRNA and 18S rRNA in a urine cell sample from the patient. Such a method can include using a logistic regression model to evaluate CD3ε mRNA, IP-10 mRNA, and 18S rRNA expression levels, or a weighted combination of log transformed, normalized RNA levels of CD3ε mRNA, IP-10 mRNA, and 18S rRNA based on a logistic regression model. The log transformation or RNA levels substantially reduces a positive skew in the data.

A method that includes measuring RNA expression levels can include normalizing the determined amounts of CD3ε mRNA and IP-10 mRNA against the amount of 18S rRNA in the sample, to generate 18S rRNA-normalized CD3ε mRNA and 18S rRNA-normalized IP-10 mRNA. The level of gene expression can be determined using log-transformed RNA levels determined by normalizing mRNA levels to 18S rRNA using a logistic regression model of CD3ε mRNA, IP-10 mRNA and 18S rRNA or a weighted combination of log transformed, normalized RNA levels of CD3ε mRNA, IP-10 mRNA and 18S rRNA based on a logistic regression model. Logistic regression models are used for prediction of the probability of occurrence of acute rejection by fitting data to a logistic curve. It is a generalized linear model used for binomial regression.

In some embodiments, for interpretation of quantitative gene expression measurements, a normalizer may be used to correct expression data for differences in cellular input, RNA quality, and RT efficiency between samples. In some embodiments, to accurately assess whether increased RNA is significant, the gene expression can be normalized to accurately compare levels of expression between samples, for example, between a baseline (control) level and an expression level detected in a test sample. In quantitative assays, such as for example, quantitative real-time Reverse Transcriptase-PCR (RT-PCR) normalization can be performed using housekeeping genes (e.g., 18S rRNA) as references against the expression level of a gene under investigation. Normalization includes rendering the measurements of different arrays or PCR or in particular RT-PCR experiments comparable by reducing or removing the technical variability. Within these experiments there exists a multiplicity of sources capable of falsifying the measurements. Possible technical sources of interference are: different efficiency in reverse transcription, labeling or hybridization reactions, as well as problems with the arrays, batch effects in reagents, or lab-specific conditions. A more robust detection of gene expression can occur when normalization is employed.

Normalization can involve use of a “housekeeping gene” which is utilized as a reference, internal control or reference value in the quantification of gene expression. The housekeeping gene allows an identification and quantitative analysis of a gene whose activity is regulated differentially in different pathological conditions. A housekeeping gene exhibits minimum change of expression and transcription across different RNA samples and thus serves as a control, or reference, for the measurement of variable gene activities across different samples. Housekeeping genes for RNA detection include, for example, 2-Microglobulin 032M), Glucose-6-phosphate dehydrogenase (G6PDH), 5-aminolevulinate synthase (ALAS or ALAS 1) Hypoxanthinephophoribosyltransferase (HPRT), Porphobilinogen deaminase (PBGD), 18S rRNA, or the like. Various housekeeping genes and normalization reagents are available from many sources including Applied Biosystems, (Foster City, Calif.), and geNorm® kits Hoffmann-La Roche (Nutley, N.J.).

In some embodiments, 18S rRNA is used for normalization in gene expression analysis. For example, the values of 18S rRNA-normalized CD3c mRNA and 18S rRNA-normalized IP-10 mRNA can be used in the diagnostic signature provided herein.

The rationale for the use of 18S rRNA to normalize mRNA is as follows. The strategy of absolute quantification of mRNA abundance rather than relative quantification (wherein the reference gene abundance is integral to the calculation of target gene abundance) facilitated the identification that 18S rRNA levels are higher in urine specimens from the acute cellular rejection biopsy group compared to the No Rejection biopsy group or the Stable (no biopsy) group. One consequence of 18S rRNA levels being higher is that 18S rRNA normalization of the target leads to underestimation of the increase in the target gene abundance in urine specimens from the acute cellular rejection biopsy group. This was seen from the finding that that the levels of CD103 (P<0.0001), CXCR3 (P<0.0001), PI-9 (P=0.002) and TGF-β1 (P=0.0001) are all significantly different between the acute cellular rejection biopsy group and the No Rejection biopsy group when non-normalized levels are used (see, e.g., U.S. Ser. No. 14/170,132, Table 5B, analysis using non-normalized levels).

The reasons for the use of 18S rRNA levels to normalize target gene levels include: (i) to ensure that the differences in the levels of a target gene between the acute cellular rejection biopsy group and the No Rejection biopsy group or Stable (no biopsy) group are not due to reasons such as variations in RNA integrity and reverse transcription efficiency, with normalization serving as an internal control; and (ii) to demonstrate that increased levels of specific mRNAs (e.g., IP-10) in association with acute cellular rejection are above and beyond the increase in the transcriptional machinery of the cell from which the total RNA was isolated. The data provided in U.S. Ser. No. 14/170,132 (see, e.g., Table 5B) show that 18S rRNA levels are about 2-fold higher in the acute cellular rejection biopsy group compared to the No Rejection biopsy group or the Stable (no biopsy) group (similar to the increases seen in CD103, CXCR3, PI-9, and TGF-β1) whereas the levels of mRNA for CD3ε, granzyme B, perforin and IP-10 are 10-fold higher or more in the acute cellular rejection biopsy group compared to the No Rejection biopsy group or the Stable (no biopsy) group. Also, scholarly guidelines on the reporting of real-time PCR experiments consider normalization to be an “essential component of a reliable PCR assay” (The MIQE Guidelines: Minimum Information for Publication of Quantitative Real-Time PCR Experiments; Bustin et al., Clin Chem 55:611-22 (2009)).

The basis for 18S rRNA levels being about 2-fold higher in the acute cellular rejection biopsy group compared to the No Rejection biopsy group or the Stable (no biopsy) group may reflect more than cell activation alone. It is however unlikely that the increase is due to a higher number of cells since irrespective of the RNA yield from a given cell pellet, reverse transcription of RNA to cDNA is adjusted to result in a final concentration of 1.0 microgram of cDNA in 100 μl solution prior to measurement of transcript abundance. This normalization of RNA yield from different urinary cell pellets, that is one urine cell pellet containing 1×10⁶ urinary cells yielding a higher amount of RNA compared to another urinary cell pellet containing 1×10⁵ urinary cells, would tend to minimize the cell number dependent differences in 18S rRNA abundance and for that matter all of the mRNAs measured in this study. On the other hand, differences in the types of cells contributing to the cell pellet could contribute to differences in transcript abundance. It is known that 18S rRNA abundance is lower in highly differentiated cells compared to less differentiated cells (Lodish, Annual Review of Biochemistry 45:39-72 (1976)), and a urine cell pellet that contains mostly highly differentiated renal tubular cells compared to a cell pellet that contains mostly activated lymphocytes would be expected to have lesser amounts of 18S rRNA.

Generally, the level of CD3ε mRNA, IP-10 mRNA and 18S rRNA in a sample is upregulated if the gene expression of CD3ε mRNA, IP-10 mRNA and 18S rRNA is increased. In some embodiments, upregulation includes increases above a control or baseline level of 10%, 15%, 20%, 25%, 30%, 35%, 40%, 45%, 50%, 55%, 60%, 65%, 70%, 75%, 80%, 85%, 90%, 95%, 100% or higher.

For example, a discriminatory level for upregulated gene expression (e.g., the baseline magnitude of gene expression) of CD3ε mRNA, IP-10 mRNA and 18S rRNA includes the mean+95% confidence interval of a group of values observed in non-rejecting transplants (e.g., baseline levels or control levels). Upregulation of CD3ε mRNA, IP-10 mRNA and 18S rRNA expression is considered to be significantly greater if the value is greater than the mean+95% confidence interval of a group of values observed in non-rejecting transplants. Similarly, the level of CD3ε mRNA, IP-10 mRNA and 18S rRNA in the sample is considered to be significantly lower if the amount of CD3ε mRNA, IP-10 mRNA and 18S rRNA detected is lower than the mean±95% confidence interval of the amount detected in non-rejecting transplants.

The method described in U.S. Ser. No. 14/170,132 for detecting or predicting kidney dysfunction based on RNA expression levels can be used herein in conjunction with the method for detecting or predicting transplant rejection based on urinary metabolite levels. A method that involves evaluation of RNA expression levels can include steps of measuring amounts of CD3ε mRNA, IP-10 mRNA and 18S rRNA in a sample; and comparing the amount of CD3ε mRNA, IP-10 mRNA and 18S rRNA in the sample to a control or baseline amount of CD3ε mRNA, IP-10 mRNA and 18S rRNA, wherein increases between the amount of CD3ε mRNA, IP-10 mRNA and 18S rRNA in the sample relative to the control indicates that the subject can have or can develop kidney dysfunction (e.g., acute cellular rejection).

### Prediction of Transplant Rejection

Methods described herein can detect or predict kidney dysfunction (e.g., acute cellular rejection) a number of days prior to acute rejection. For example, the methods can detect or predict kidney dysfunction (e.g., acute cellular rejection) about 15 to 90 days before transplant rejection. In some case, the methods can detect or predict kidney dysfunction (e.g., acute cellular rejection) 90 to 60 days before confirmation by biopsy, or 59 to 30 days before confirmation by biopsy, or 29 to 16 days before confirmation by biopsy, or 4 to 30 days before confirmation by biopsy. Thus, kidney transplant dysfunction such as acute cellular rejection can be predicted about 3 months to about four days before it happens. Such RNA profile analysis can be a new non-invasive “gold standard” to replace and/or supplement an invasive allograft biopsy.

In some embodiments, the method for predicting acute rejection employs log-transformed RNA values determined from an urine sample and that are determined by combinations of log transformed and/or normalized RNA values using a logistic regression model of CD3ε mRNA, IP-10 mRNA, and 18S rRNA expression levels, which predict acute rejection of the transplanted organ within about 90 to about 60 days after the urine sample is tested. In some embodiments; the log-transformed RNA levels of the urine sample are determined by combinations of log transformed, normalized RNA levels using a logistic regression model of CD3ε mRNA, IP-10 mRNA, and 18S rRNA expression levels that predicts acute rejection of the transplanted organ in about 59 to about 30 days after the urine sample is tested. In some embodiments, the log-transformed RNA levels of the urine sample are determined by combinations of log transformed, normalized CD3ε mRNA, IP-10 mRNA, and 18S rRNA expression levels that predict acute rejection of the transplanted organ in about 29 to about 15 days after the urine sample is tested.

Quantified expression levels of 18S rRNA-normalized CD3ε mRNA, 18S rRNA-normalized IP-10 mRNA, and 18S rRNA can be used in the diagnostic signature algorithm provided herein. In some instances, the values of 18S-normalized CD3ε mRNA, 18S-normalized IP-10 mRNA and 18S rRNA can deviate from a normal distribution (P<0.001). Log₁₀-transformation of these values can substantially reduce this deviation.

Moreover, 18S rRNA can be expressed at higher levels than CD3ε mRNA and IP-10 mRNA. Hence, the quantified amount of 18S rRNA in the diagnostic signature algorithm can be adjusted by a factor of 10⁻⁶ to allow better relationship between the expression levels of these three genes.

### Serum Creatinine

The method can further comprise determining the patient's serum creatinine protein level. The determination of the level of serum creatinine can be made by any method known to those skilled in the art. The next step in this embodiment can include correlating the level of serum creatinine in peripheral blood with predicting acute rejection and eventual loss of the transplanted organ. A significantly greater level of serum creatinine in peripheral blood and increased levels of CD3ε mRNA, IP-10 mRNA, and 18S rRNA in urine correlates with acute rejection and may also increase risk of loss of the transplanted kidney.

Generally, the level of serum creatinine in peripheral blood is considered to be significantly greater if the level is at least about 25% greater than the level of creatinine in a control sample. Commercial kits can be utilized to test creatinine. An example of a commercial kit for determining creatinine level is the QuantiChrom® Creatinine Assay Kit from BioAssay Systems (Hayward, Calif.).

A control sample can be the level of serum creatinine in peripheral blood of a healthy person or a person with a well-functioning (e.g., stable) transplant. For example, the normal level of serum creatinine in a healthy person or a person with a well-functioning transplant is generally about 0.8-1.6 milligrams/deciliter. In either case, the person may be the patient or a person different from the patient.

It is not necessary to determine the level of creatinine in a control sample every time the method is conducted. For example, the serum creatinine level from the patient can be compared to that of one or more previously determined control samples or to a level recognized by the physician or clinician conducting the method, or by a consensus of medical and/or clinical practitioners.

### Treatment

The methods can further include informing medical personnel or the patient about the test results. Information about whether the patient will have acute rejection can also be communicated.

If a difference in 3-sialyllactose/xanthosine ratios, quinolinate/X-16397 ratios, CD3ε mRNA levels, IP-10 mRNA levels, and/or 18S rRNA levels is determined, the patient can be informed that there is increased risk of developing transplant rejection. Similarly, if any of the methods described herein that include a diagnostic signature algorithm indicate that a subject's urine sample is different from a baseline or control value, the patient can be informed that there is increased risk of developing transplant rejection. The increased risk varies in different patients, and the organ transplanted. Generally, the increased risk for developing acute rejection is at least about 25%, at least about 50%, at least about 75%, or at least about 90%, or at least about 99% or at least about 100%.

If the patient is likely to develop kidney dysfunction, the patient can be prescribed and/or administered a treatment to delay or obviate rejection of the transplanted organ. Such treatment can include increased or decreased dose of an anti-rejection agent or an anti-rejection agent can be added. Anti-rejection agents, include for example, azathioprine, cyclosporine, FK506, tacrolimus, mycophenolate mofetil, anti-CD25 antibody, antithymocyte globulin, rapamycin, ACE inhibitors, perillyl alcohol, anti-CTLA4 antibody, anti-CD40L antibody, anti-thrombin III, tissue plasminogen activator, antioxidants, anti-CD 154, anti-CD3 antibody, thymoglobin, OKT3, corticosteroid, or a combination thereof.

For example, if acute rejection is predicted or ongoing, a steroid pulse therapy can be started and may include the administration for three to six days of a high dose corticosteroid (e.g., greater than 100 mg). An antibody can be added. An example of an antibody therapy includes the administration for seven to fourteen days of the polyclonal antibody Thymoglobin or the monoclonal antibody, OT3.

Another example of a treatment that can be administered is plasmapheresis. Plasmapheresis is a process in which the fluid part of the blood (i.e., plasma) is removed from blood cells. Typically, the plasma is removed by a device known as a cell separator. The cells are generally returned to the person undergoing treatment, while the plasma, which contains antibodies, is discarded.

The implications of immunosuppressive therapy relating to the diagnostic signatures disclosed herein are as follows. The lower trajectory (lower diagnostic score) in those induced with T cell depleting antibodies compared to subjects induced with non-depleting antibodies is consistent with T cell depleting antibodies inducing a greater degree of immunosuppression and being associated with a lower incidence of acute rejection as compared to IL-2 receptor antagonists. The findings described herein and U.S. Ser. No. 14/170,132, in addition to suggesting a mechanistic basis for the lower incidence of acute rejection for induction with depleting antibodies vs. non-depleting antibodies, indicate that the signature can serve as an accurate indicator of the kidney graft recipient's immune status and help personalize immunosuppressive therapy.

The impact of anti-rejection therapy on the diagnostic signature is as follows. A comparison of the diagnostic signature values showed a significant decrease following treatment of an episode of acute cellular rejection. While this decrease in the mean value of the diagnostic signature remained statistically significant in the subset of patients who responded to therapy, almost 30% of the responders did not show a numerical decrease in the diagnostic signature. (The number of non-responders to acute cellular rejection treatment [N=4] was too small to yield meaningful results.) These findings indicate that a successful clinical response is associated with a reduction or normalization of the signature in many, but not in all cases. This lack of complete resolution of the molecular signature is reminiscent of the previously published finding wherein almost two-thirds of kidney graft recipients with clinical reversal of acute rejection had residual inflammation in their follow-up graft biopsies (Gaber et al., Kidney Int 55:2415-22 (1999)). In the U.S. double blind, randomized, multicenter, phase III clinical trial of Thymoglobulin versus ATGAM in the treatment of acute graft rejection episodes after renal transplantation, a subset of patients (n=38) had both a baseline inclusion biopsy and a protocol biopsy one to two weeks following the end of therapy and it was found that “treatment was clinically successful in 94% of patients receiving Thymoglobulin and in 79% of patients receiving ATGAM, but only 35% and 33% of the respective biopsies demonstrated total resolution of the rejection on the repeat biopsies” (Gaber et al., Kidney Int 55:2415-22 (1999)).

The implications of infections upon the diagnostic signatures described herein are as follows. The data demonstrate that urinary tract infection, blood infection and CMV are not associated with the diagnostic score, giving strong assurance that these types of infections will not impact the diagnostic accuracy of the signature. Thus, these infections will not increase the diagnostic signature score and result in a false positive acute cellular rejection diagnosis. On the other hand, the association of BKV infection with the signature suggests that the presence or absence of BKV infections needs to be confirmed prior to ascribing an elevated diagnostic signature score to acute cellular rejection. In the clinical setting, this can and should be accomplished by screening for BKV in blood or urine, a routine clinical practice in many transplant centers.

### Kits

The methods can also be performed by use of kits that are described herein. In general, kits can include a detection reagent that is suitable for detecting the presence of a metabolite or an RNA of interest.

The kits can include a panel of probe and/or primer sets. Such probe and/or primer sets are designed to detect expression of one or more genes and provide information about the rejection of a graft. Preferred probe sets comprise probes or primers that can be labeled (e.g., fluorescer, quencher, etc.). Unlabeled probes or primers can also be provided in the kits.

The probes and primers are useful for detection of CDR mRNA, IP-10 mRNA, and 18S rRNA. The probe and/or primer sets are targeted at the detection of gene transcripts that are informative about acute rejection. Probe and/or primer sets may also comprise a large or small number of probes or primers that detect gene transcripts that are not informative about transplant rejection. Such probes and primers are useful as controls and for normalization. Probe and/or primer sets can be provided in the kits as a dry material or dissolved in solution. In some embodiments, probe and/or primer sets can be affixed to a solid substrate to form an array of probes. Probe and/or primer sets can be configured for multiplex PCR. The probes and/or primers can be nucleic acids (e.g., DNA, RNA, chemically modified forms of DNA and RNA), LNA, or PNA, or any other polymeric compound capable of specifically interacting with the desired nucleic acid sequences.

The kits can include components for isolating and/or detecting mRNA in essentially any sample (e.g., urine, blood, etc.), and a wide variety of reagents and methods are, in view of this specification, known in the art. Hence, the kits can include vials, swabs, needles, syringes, labels, pens, pencils, or combinations thereof.

Commercially available components can also be included in the kits.

For example, the kit can include components from QIAGEN, which manufactures a number of components for RNA isolation, including RNEASY, a Total RNA System (involving binding total RNA to a silica-gel-based membrane and spinning the RNA); OLIGOTEX® for isolation of RNA utilizing spherical latex particles; and QIAGEN total RNA kit for In Vitro Transcripts and RNA clean-up.

The kits can include components for a fluorescence based real-time detection method. For example, the kits can include primers for generating cDNA and/or for amplification of mRNA and rRNA. The kits can include components for 5′ nuclease assays employ oligonucleotide probes labeled with at least one fluorescer and at least one quencher. Prior to cleavage of the probe, the fluorescer excites the quencher(s) rather than producing a detectable fluorescence emission. The oligonucleotide probe hybridizes to a target oligonucleotide sequence for amplification in PCR. The nuclease activity of the polymerase used to catalyze the amplification of the primers of the target sequence serves to cleave the probe, thereby causing at least one fluorescer to be spatially separated from the quencher so that the signal from the fluorescer is no longer quenched. A change in fluorescence of the fluorescer and/or a change in fluorescence of the quencher due to the oligonucleotide probe being digested can be used to indicate the amplification of the target oligonucleotide sequence. Although some primers and probes are described in Table 4, other suitable primers and probes can be employed. Probes and primers can be designed using techniques available to those of skill in the art.

The kits can also include any of the following components: materials for obtaining a sample, enzymes, buffers, probes, primers for generating cDNA, primers for amplifying RNA or cDNA, materials for labeling nucleic acids, microarrays, one or more microarray reader, competitor nucleic acids, probes and/or primers for a housekeeping gene for normalization, control nucleic acids, and antibodies.

In further embodiments, kits can include a urine collection system. Urine collection systems can include essentially any material useful for obtaining and/or holding a urine sample. Urine collection systems may include, for example, tubing, a beaker, a flask, a vial, a test tube, a container, and/or a lid for a vial, test tube or container (e.g., a plastic container with a snap-on or screw top lid).

In certain embodiments, kits can also include a urine presentation system. A urine presentation system can include essentially any material that is useful for presenting the urine to be contacted with the appropriate detection or purification reagents. A urine presentation system may comprise, for example, a sample well, which may be part of a multi-well plate, a petri dish, a filter (e.g., paper, nylon, nitrocellulose, PVDF, cellulose, silica, phosphocellulose, or other solid or fibrous surface), a microchannel (which may be part of a microchannel array or a microfluidics device), a small tube such as a thin-walled PCR tube or a 1.5 ml plastic tube, a microarray to which urine or material obtained from urine may be applied, a capillary tube or a flat or curved surface with detection reagent adhered thereto, or a flat or curved surface with material that adheres to proteins or nucleic acids present in the urine sample.

Kits can include probes that may be affixed to a solid surface to form a customized array.

Kits may also comprise a sample preparation system. A sample preparation system comprises, generally, any materials or substances that are useful in preparing the urine sample to be contacted with the detection reagents. For example, a sample preparation system may comprise materials for separating urine sediments from the fluids, such as centrifuge tube, a microcentrifuge, or a filter (optionally fitted to a tube designed to permit a pressure gradient to be established across the filter). One example of a filter that can be used is a filter within a syringe, such as those available from Zymo Research (see website at zymoresearch.com/columns-plastics/column-filter-assemblies/zrc-gf-filter; e.g., ZRC-GF Filter™). Other components that can be included in the kit include buffers, precipitating agents for precipitating either wanted or unwanted materials, chelators, cell lysis reagents, RNase inhibitors etc.

Collection, presentation and preparation systems can accomplished in various ways. For example, a filter can be used to separate urine sediments from the fluids, and the filter may be coated with antibodies suitable for specifically detecting the desired proteins. One of skill in the art would, in view of this specification, readily understand many combinations of components that a kit of the invention may comprise.

### Definitions

An “anti-rejection agent” is any substance administered to a subject for the purpose of preventing or ameliorating a rejection state. Anti-rejection agents include, but are not limited to, azathioprine, cyclosporine, FK506, tacrolimus, mycophenolate mofetil, anti-CD25 antibody, antithymocyte globulin, rapamycin, ACE inhibitors, perillyl alcohol, anti-CTLA4 antibody, anti-CD40L antibody, anti-thrombin III, tissue plasminogen activator, antioxidants, anti-CD 154, anti-CD3 antibody, thymoglobin, OKT3, corticosteroid, or a combination thereof. “Baseline therapeutic regimen” is understood to include those anti-rejection agents being administered at a baseline time, subsequent to the transplant. The baseline therapeutic regimen may be modified by the temporary or long-term addition of other anti-rejection agents, or by a temporary or long-term increase or decrease in the dose of one, or all, of the baseline anti-rejection agents.

The term “biopsy” refers to a specimen obtained by removing tissue from living patients for diagnostic examination. The term includes aspiration biopsies, brush biopsies, chorionic villus biopsies, endoscopic biopsies, excision biopsies, needle biopsies (specimens obtained by removal by aspiration through an appropriate needle or trocar that pierces the skin, or the external surface of an organ, and into the underlying tissue to be examined), open biopsies, punch biopsies (trephine), shave biopsies, sponge biopsies, and wedge biopsies. Biopsies also include a fine needle aspiration biopsy, a minicore needle biopsy, and/or a conventional percutaneous core needle biopsy.

A “sample” includes fluid samples obtained from a subject. A sample can contain metabolites, cells, proteins, nucleic acids or other cellular matter. A sample for analysis of metabolites may be the liquid phase of a body fluid from which sedimentary materials have been substantially removed. A sample for analysis of RNA may be the sedimentary materials from centrifugation of a sample. Exemplary samples include, but are not limited to, blood samples containing peripheral blood mononuclear cells (PBMCs), urine samples containing urinary cells, urine “supernatant” that is substantially free of cells, a sample of bronchoalveolar lavage fluid, a sample of bile, pleural fluid or peritoneal fluid, or any other fluid secreted or excreted by a normally or abnormally functioning allograft, or any other fluid resulting from exudation or transudation through an allograft or in anatomic proximity to an allograft, or any fluid in fluid communication with the allograft. A sample can be directly obtained from a subject or it can be obtained indirectly, for example, after retrieval, transport and/or storage of the sample by another. A sample may also be obtained from essentially any body fluid including: urine, blood (including peripheral blood), lymphatic fluid, sweat, peritoneal fluid, pleural fluid, bronchoalveolar lavage fluid, pericardial fluid, gastrointestinal juice, bile, feces, tissue fluid or swelling fluid, joint fluid, cerebrospinal fluid, or any other named or unnamed fluid gathered from the anatomic area in proximity to the allograft or gathered from a fluid conduit in fluid communication with the allograft.

A “post-transplantation sample” refers to a sample obtained from a subject after the transplantation has been performed.

“Baseline level of gene expression level” includes the particular gene expression level of a healthy subject or a subject with a well-functioning transplant. The baseline level of gene expression includes the gene expression level of a subject without acute rejection. The baseline level of gene expression can be a number on paper or the baseline level of gene expression from a control sample of a healthy subject or a subject with a well-functioning transplant.

The term “determining” is used herein to mean testing, assaying, and/or physically manipulating a sample to ascertain what the sample contains. In some cases, “determining” can also include quantifying a component of a sample.

The term “diagnosis” is used herein to refer to the identification or classification of a molecular or pathological state, disease or condition. For example, “diagnosis” may refer to identification of a particular type of acute rejection, e.g., acute cellular rejection.

The term “aiding diagnosis” is used herein to refer to methods that assist in making a clinical determination regarding the presence, degree or other nature, of a particular type of symptom or condition of acute rejection.

The term “prediction” or “predicting” is used herein to refer to the likelihood that a patient will develop acute rejection. Thus, prediction also includes the time period without acute rejection.

A “probe or primer” as used herein refers to a group of nucleic acids that may be used to detect one or more genes (e.g., 18S rRNA, CD3ε, and IP-10). Detection may be, for example, through amplification as in PCR, QPCR, RT-PCR, or primer extension. Detection can also be through hybridization, or through selective destruction and protection, as in assays based on the selective enzymatic degradation of single or double stranded nucleic acids, or by detecting RNA affixed to a solid surface (e.g., a blot). Probes and/or primers may be labeled with one or more fluorescent labels, radioactive labels, fluorescent quenchers, enzymatic labels, or other detectable moieties. Probes may be any size so long as the probe is sufficiently large to selectively detect the desired nucleic acid or to serve as a primer for amplification.

Primers can be polynucleotides or oligonucleotides capable of being extended in a primer extension reaction at their 3′ end. In order for an oligonucleotide to serve as a primer, it typically needs only be sufficiently complementary in sequence to be capable of forming a double-stranded structure with the template, or target, under the conditions employed. Establishing such conditions typically involves selection of solvent and salt concentration, incubation temperatures, incubation times, assay reagents and stabilization factors known to those in the art. The term primer or primer oligonucleotide refers to an oligonucleotide as defined herein, which is capable of acting as a point of initiation of synthesis when employed under conditions in which synthesis of a primer extension product that is complementary to a nucleic acid strand is induced, as, for example, in a DNA replication reaction such as a PCR reaction. Like non-primer oligonucleotides, primer oligonucleotides may be labeled according to any technique known in the art, such as with radioactive atoms, fluorescent labels, enzymatic labels, proteins, haptens, antibodies, sequence tags, mass label or the like. Such labels may be employed by associating them, for example, with the 5′ terminus of a primer by a plurality of techniques known in the art. Such labels may also act as capture moieties. A probe or primer may be in solution, as would be typical for multiplex PCR, or a probe or primer may be adhered to a solid surface, as in an array or microarray. It is well known that compounds such as PNAs may be used instead of nucleic acids to hybridize to genes. In addition, probes may contain rare or unnatural nucleic acids such as inosine.

As used herein, the term polynucleotide or nucleic acid includes nucleotide polymers of any number. The term polynucleotide includes a molecule comprising any number of nucleotides, preferably, less than about 200 nucleotides. More preferably, polynucleotides are between 5 and 100 nucleotides in length. Most preferably, polynucleotides are 15 to 100 nucleotides in length. The exact length of a particular polynucleotide, however, will depend on many factors, which in turn depend on its ultimate function or use. Some factors affecting the length of a polynucleotide are, for example, the sequence of the polynucleotide, the assay conditions in terms of such variables as salt concentrations and temperatures used during the assay, and whether or not the polynucleotide is modified at the 5′ terminus to include additional bases for the purposes of modifying the mass: charge ratio of the polynucleotide, or providing a tag capture sequence which may be used to geographically separate a polynucleotide to a specific hybridization location on a DNA chip, for example.

As used herein, the term “transplantation” refers to the process of taking a cell, tissue, or organ, called a “transplant” or “graft” from one individual and placing it or them into a (usually) different individual. The individual who provides the transplant is called the “donor” and the individual who received the transplant is called the “recipient” (or “host”). An organ, or graft, transplanted between two genetically different individuals of the same species is called an “allograft.” A graft transplanted between individuals of different species is called a “xenograft.”

As used herein, “transplant rejection” or “allograft rejection” refers to a functional and structural deterioration of the organ due to an active immune response expressed by the recipient, and independent of non-immunologic causes of organ dysfunction. Acute transplant rejection can result from the activation of recipient's T cells and/or B cells; the rejection primarily due to T cells is classified as T cell mediated acute rejection or acute cellular rejection (ACR) and the rejection in which B cells are primarily responsible is classified as antibody mediated acute rejection (AMR). In some embodiments, the methods and compositions provided can detect and/or predict acute cellular rejection.

As used herein, “subject” means a mammal. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; or the like. The term “subject” does not denote a particular age or sex. Preferably the subject is a human patient. In some embodiments, the subject is a human who has received an organ transplant.

The term “up-regulation,” “up-regulated,” “increased expression,” and “higher expression” are used interchangeably herein and refer to the increase or elevation in the amount of a target mRNA or a target protein. In some embodiments, up-regulation,” “up-regulated,” “increased expression,” and “higher expression” includes increases above a baseline (e.g., a control, or reference) level of 10%, 15%, 20%, 25%, 30%, 35%, 40%, 45%, 50%, 55%, 60%, 65%, 70%, 75%, 80%, 85%, 90%. 95%, 100% or higher.

The term “hybridization” includes a reaction in which one or more nucleic acids or polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, primer extension reaction, or the enzymatic cleavage of a polynucleotide by a ribozyme.

As used herein, the terms “hybridize” and “hybridization” refer to the annealing of a complementary sequence to the target nucleic acid, i.e., the ability of two polymers of nucleic acid (polynucleotides) containing complementary sequences to anneal through base pairing. The terms “annealed” and “hybridized” are used interchangeably throughout, and are intended to encompass any specific and reproducible interaction between a complementary sequence and a target nucleic acid, including binding of regions having only partial complementarity. Certain bases not commonly found in natural nucleic acids may be included in the nucleic acids of the present invention and include, for example, inosine and 7-deazaguanine. Those skilled in the art of nucleic acid technology can determine duplex stability empirically considering a number of variables including, for example, the length of the complementary sequence, base composition and sequence of the oligonucleotide, ionic strength and incidence of mismatched base pairs. The stability of a nucleic acid duplex is measured by the melting temperature, or “Tₘ”. The Tₘ of a particular nucleic acid duplex under specified conditions is the temperature at which on average half of the base pairs have disassociated.

Hybridization reactions can be performed under conditions of different “stringency”. The stringency of a hybridization reaction includes the difficulty with which any two nucleic acid molecules will hybridize to one another. Under stringent conditions, nucleic acid molecules at least 60%, 65%, 70%, 75% identical to each other remain hybridized to each other, whereas molecules with low percent identity cannot remain hybridized. A preferred, non-limiting example of highly stringent hybridization conditions are hybridization in 6×sodium chloride/sodium citrate (SSC) at about 45° C., followed by one or more washes in 0.2×SSC, 0.1% SDS at 50° C., preferably at 55° C., more preferably at 60° C., and even more preferably at 65° C. When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide if hybridization can occur between one of the strands of the first polynucleotide and the second polynucleotide.

“Complementarity” or “homology” is quantifiable in terms of the proportion of bases in opposing strands that are expected to hydrogen bond with each other, according to generally accepted base-pairing rules.

The term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “medium” or “low” stringency are often required when it is desired that nucleic acids which are not completely complementary to one another be hybridized or annealed together. The art knows well that numerous equivalent conditions can be employed to comprise medium or low stringency conditions. The choice of hybridization conditions is generally evident to one skilled in the art and is usually guided by the purpose of the hybridization, the type of hybridization (DNA-DNA or DNA-RNA), and the level of desired relatedness between the sequences (e.g., Sambrook et al. (1989); Nucleic Acid Hybridization, A Practical Approach, IRL Press, Washington D.C. 1985, for a general discussion of the methods).

The stability of nucleic acid duplexes is known to decrease with an increased number of mismatched bases, and further to be decreased to a greater or lesser degree depending on the relative positions of mismatches in the hybrid duplexes. Thus, the stringency of hybridization can be used to maximize or minimize stability of such duplexes. Hybridization stringency can be altered by: adjusting the temperature of hybridization; adjusting the percentage of helix destabilizing agents, such as formamide, in the hybridization mix; and adjusting the temperature and/or salt concentration of the wash solutions. For filter hybridizations, the final stringency of hybridizations often is determined by the salt concentration and/or temperature used for the post-hybridization washes.

“High stringency conditions” when used in reference to nucleic acid hybridization include conditions equivalent to binding or hybridization at 42° C. in a solution consisting of 5×SSPE (43.8 g/l NaCl, 6.9 g/1NaH₂PO₄ H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed. In general, the stringency of hybridization is determined by the wash step. Hence, a wash step involving 0.1×SSPE, 1.0% SDS at a temperature of at least 42° C. can yield a high stringency hybridization product. In some instances the high stringency hybridization conditions include a wash in 1×SSPE, 1.0% SDS at a temperature of at least 50° C., or at about 65° C.

“Medium stringency conditions” when used in reference to nucleic acid hybridization include conditions equivalent to binding or hybridization at 42 □° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed. Hence, a wash step involving 1.0×SSPE, 1.0% SDS at a temperature of 42° C. can yield a medium stringency hybridization product.

“Low stringency conditions” include conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharmacia), 5 g BSA (Fraction V; Sigma)] and 100 g/ml denatured salmon sperm DNA followed by washing in a solution comprising 5×SSPE, 0.1% SDS at 42° C. when a probe of about 500 nucleotides in length is employed. Hence, a wash step involving 5×SSPE, 1.0% SDS at a temperature of 42° C. can yield low stringency hybridization product.

A “gene product” includes a peptide, polypeptide, or structural RNA generated when a gene is transcribed and/or translated. While an mRNA encoding a peptide or polypeptide can be translated to generate the peptide or polypeptide, a structural RNA (e.g., an rRNA) is not translated. In some embodiments, the target gene expresses 18S rRNA, CD3c, and IP-10.

The term “level of gene expression” as used herein refers to quantifying gene expression. In some embodiments, to accurately assess whether increased mRNA or rRNA is significant, it is preferable to “normalize” gene expression to accurately compare levels of expression between samples, i.e., it is a baseline level against which gene expression is compared. Quantification of gene expression can be accomplished by methods known in the art, such as, for example, reverse transcription polymerase chain reaction (RT-PCR), TAQMAN® assays or the like. Gene expression can also be quantified by detecting a protein, peptide or structural RNA gene product directly, in a variety of assay formats known to those of ordinary skill in the art. For example, proteins and peptides can be detected by an assay such as an enzyme linked immunosorbent assay (ELISA), radioimmunoassay (RIA), immunofluorimetry, immunoprecipitation, equilibrium dialysis, immunodiffusion, immunoblotting, mass spectrometry and other techniques. See, e.g., Harlow and Lane, Antibodies: A Laboratory Manual, Cold Spring Harbor Laboratory, 1 88; Weir, D. M., Handbook of Experimental Immunology, 1986, Blackwell Scientific, Boston.

As used herein, the term “biomarker” includes a polynucleotide or polypeptide molecule which is present or increased in quantity or activity in subjects having acute rejection or where the acute rejection is anticipated.

As used herein, the term “panel of biomarkers” includes a group of markers, the quantity or activity of each member of which is correlated with subjects having acute rejection or where the acute rejection is anticipated. In certain embodiments, a panel of markers may include only those markers which are either increased in quantity or activity in those subjects. In some embodiments, the panel of markers include 18S rRNA, CD3-epsilon, and IP-10.

The following non-limiting Examples describe some of the experiments performed in the development of the invention.

### Example 1: Materials and Methods

This Example describes some of the materials and methods employed in various experiments.

### Urine Sample Collection

Four hundred and eighty five patients with end stage renal disease were enrolled in the parent CTOT-04 multicenter, prospective observational study (Suthanthiran et al., N Engl J Med 369: 20-31 (2013)). Five clinical sites enrolled the CTOT-04 study participants.

Fifty to 100 ml of midstream urine were collected in a sterile, sealed urine collection cup without addition of any preservatives. Urine was processed within 1 hour of collection and if this was not possible, urine was refrigerated at 4° C. for a maximum time of 4 hours. The urine sample was centrifuged at 2000 g at room temperature for 30 minutes in sterile disposable tubes. After centrifugation, 6 ml of the supernatant was aspirated using a pipette without disturbing the cell pellet and transferred to four (4) 1.5 ml microcentrifuge polypropylene tubes.

The supernatants and the cell pellets were stored as specified by the NIH-sponsored Statistical Analysis and Clinical Coordinating Center. In brief, the supernatants and the cell pellets from the urine specimens were first stored at the clinical site at −80° C. and shipped in a Styrofoam container with dry ice. The samples from the −80° C. freezer were transferred into the middle of the dry ice to avoid any thawing of the samples. The samples were then covered with more dry ice so that the samples were completely surrounded by at least 10 cm of dry ice on all sides. The Styrofoam container was covered with a well-fitting cover and the box was sealed with duct tape. The samples were shipped to Weill Cornell Medical College Core Laboratory and stored at −80° C.

Sequential urine samples were collected from the study participants on post-transplant days 3, 7, 15 and 30 and in months 2, 3, 4, 5, 6, 9 and 12 and at the time of biopsy. A total of 4300 urine samples were prospectively collected from 485 kidney graft recipients (patients), and urine pellet and cell free supernatants were prepared at each clinical site using the standard protocol summarized above. Non-targeted metabolomics and targeted metabolite measurements were performed on aliquots of supernatants that were never thawed prior to metabolite analysis.

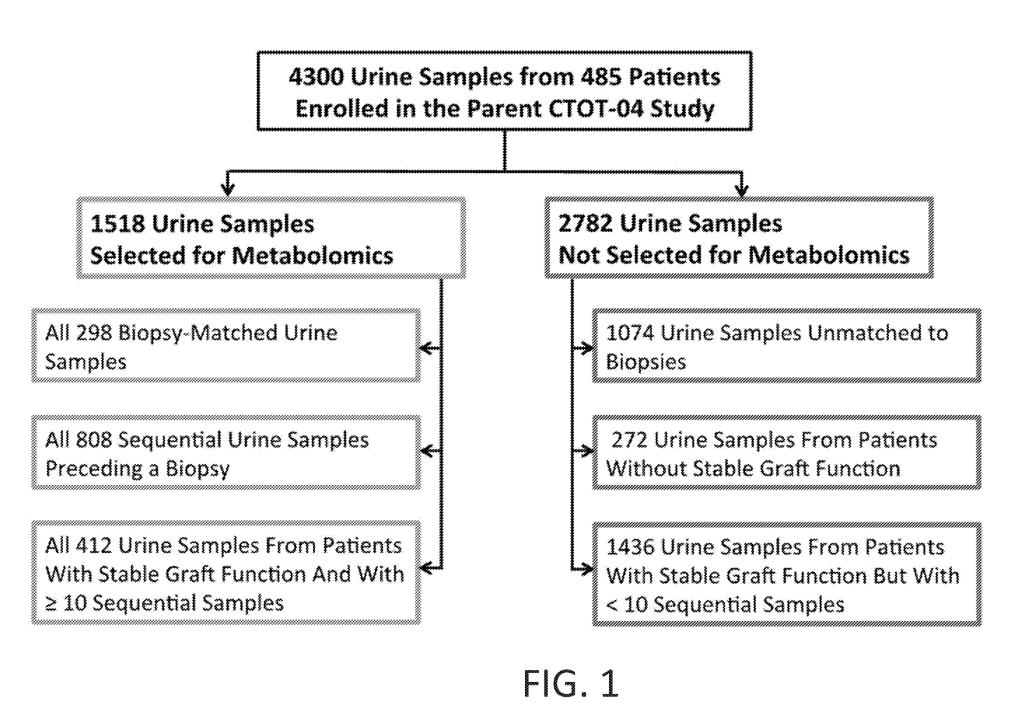

FIG. 1 provides a schematic diagram illustrating sample selection for metabolite analysis. Table 1 is a summary of the characteristics of the patients included in the analysis to develop the metabolite signatures and the composite metabolite and RNA signature.

TABLE 1
Characteristics of CTOT-04 kidney transplant recipients
included in the analysis to develop metabolite/RNA signatures
discriminating ACR biopsies from No Rejection biopsies

| | | Patients | | |
|---|---|---|---|---|
| | | with | Patients with | |
| | | ACR | No Rejection | |
| | Total* | Biopsies | Biopsies | |
| | (N = 185) | (N = 36) | (N = 149) | p-value |
| Recipient | | | | |
| Characteristics | | | | |
| Age, years | | | | |
| Mean (SD) | 46.9 (12.8) | 45.1 (12.3) | 47.3 (12.9) | 0.20 |
| Median | 46 | 42 | 47 | |
| Min, Max | 0.1, 76 | 24, 72 | 0.1, 77 | |
| Gender, N (%) | | | | |
| Female | 58 (31.4)) | 9 (25.0) | 49 (32.9) | 0.87 |
| Male | 127 (68.6) | 27 (75.0) | 100 (67.1) | |
| Ethnicity, N (%) | | | | |
| Hispanic or | 28 (15.1) | 3 (8.3) | 25 (16.8) | 0.18 |
| Latino | | | | |
| Not Hispanic | 149 (80.5) | 31 (86.1) | 118 (79.2) | |
| or Latino | | | | |
| Unknown or | 8 (4.3) | 2 (5.6) | 6 (4.0) | |
| Not Reported | | | | |
| Race, N (%) | | | | |
| Black or | 68 (36.8) | 14 (38.9) | 54 (36.2) | 0.54 |
| African | | | | |
| American | | | | |
| White | 99 (53.5) | 21 (58.3) | 78 (52.4) | |
| Asian | 11 (6.0)) | 1 (2.8) | 10 (6.7) | |
| American | 0 | 0 | 0 | |
| Indian or | | | | |
| Alaskan | | | | |
| Native | | | | |
| Other | 2 (1.1) | 0 | 2 (1.1) | |
| More Than | 0 | 0 | 0 | |
| One Race | | | | |
| Unknown or | 5 (2.7) | 0 | 5 (3.4) | |
| Not Reported | | | | |
| Induction | | | | |
| Therapy, N (%) | | | | |
| IL-2 Receptor | 23 (13.1) | 7 (19.4) | 16 (11.4) | 0.83 |
| Antibody | | | | |
| CAMPATH- | 72 (40.9) | 11 (30.6) | 61 (43.6) | |
| IH | | | | |
| Thymo- | 69 (39.2) | 15 (41.7) | 54 (38.6) | |
| globulin | | | | |
| More than one | 9 (5.1) | 2 (5.6) | 7 (5.0) | |
| induction | | | | |
| therapy | | | | |
| No induction | 3 (1.7) | 1 (2.8) | 2 (1.4) | |
| Therapy | | | | |
| Missing | 9 | 0 | 9 | |
| information, N | | | | |
| BMI | | | | |
| Mean (SD) | 28.8 (6.3) | 30.1 (6.5) | 28.4 (6.2) | 0.09 |
| Median | 27.5 | 28.6 | 27.5 | |
| Min, Max | 17, 45 | 20, 43 | 17, 45 | |
| <18.5, N (%) | 55 (31.6) | 11 (33.3) | 44 (31.2) | 0.29 |
| 18.5-24.9, N | 53 (30.5) | 7 (21.2) | 46 (32.6) | |
| (%) | | | | |
| 25.0-29.9, N | 3 (1.7) | 0 | 3 (2.1) | |
| (%) | | | | |
| ≥30.0, N (%) | 63 (36.2) | 15 (45.5) | 48 (34.0) | |
| Missing | 11 | 3 | 8 | |
| eGFR | | | | |
| (mL/min)** | | | | |
| Number of | 183 | 37 | 146 | |
| Measurements | | | | |
| Mean (SD) | | 30 (17) | 43 (23) | 0.003 |
| Median | | 27 | 40 | |
| Min, Max | | 4.7, 72.3 | 3.4, 107.7 | |
| Serum | | | | |
| Creatinine | | | | |
| (mg/dL) | | | | |
| Number of | 183 | 37 | 146 | |
| Measurements | | | | |
| Mean (SD) | | 3.7 (2.9) | 2.5 (2.3) | 0.0003 |
| Median | | 2.8 | 1.9 | |
| Min, Max 1 | | .1, 13.3 | 0.7, 21.2 | |
| Donor | | | | |
| Characteristics | | | | |
| Age | | | | |
| Mean (SD) | 40.8 (14.4) | 42.4 (11.5) | 40.4 (15.1) | 0.28 |
| Median | 41 | 40 | 41 | |
| Min, Max | 0.4, 66 | 24, 65 | 0.4, 66 | |
| Missing | 3 | 0 | 3 | |
| Gender, N (%) | | | | |
| Female | 88 (47.6) | 18 (50.0) | 70 (47.0) | 0.54 |
| Male | 97 (52.4) | 18 (50.0) | 79 (53.0) | |
| Ethnicity, N (%) | | | | |
| Hispanic or | 29 (15.7) | 4 (11.1) | 25 (16.8) | 0.27 |
| Latino | | | | |
| Not Hispanic | 138 (74.6) | 29 (80.6) | 109 (58.9) | |
| or Latino | | | | |
| Unknown or | 18 (9.7) | 3 (8.3) | 15 (10.1) | |
| Not Reported | | | | |
| Race, N (%) | | | | |
| Black or | 47 (25.4) | 10 (27.8) | 37 (24.8) | 0.78 |
| African | | | | |
| American | | | | |
| White | 122 (66.0) | 24 (66.7) | 98 (65.8) | |
| Asian | 5 (2.7) | 1 (2.8) | 4 (2.7) | |
| American | 0 | 0 | 0 | |
| Indian or | | | | |
| Alaskan | | | | |
| Native | | | | |
| Other | 1 (0.5) | 0 | 1 (0.7) | |
| Unknown or | 10 (5.4) | 1 (2.8) | 9 (6.0) | |
| Not Reported | | | | |
| Source of | | | | |
| Donor, N (%) | | | | |
| Deceased | 86 (46.5) | 17 (47.2) | 69 (46.3) | 0.43 |
| Living, related | 53 (28.7) | 9 (25.0) | 44 (29.5) | |
| Living, | 46 (24.9) | 10 (27.8) | 36 (24.2) | |
| unrelated | | | | |

### Urine Sample Selection for Metabolomics.

From a total of 4300 urine samples prospectively collected from the 485-kidney allograft recipients (patients) enrolled in the parent CTOT-04 study, 1518 urine samples were selected for metabolite analysis to include the following urine samples: (1) all biopsy-matched urine samples, 298 samples matched to 298 kidney allograft biopsies performed in 190 patients (biopsy matched urine samples=urine samples collected from 3 days before to 1 day after the biopsy); (2) all 808 sequential samples from 112 patients that preceded a first biopsy classified using Banff classification schema2 as acute cellular rejection, antibody-mediated rejection, borderline changes, or other, and sequential samples that preceded No Rejection biopsies; and (3) all 412 sequential samples from 40 patients with stable graft function and who had at least 10 sequential samples collected in the first 400 days of transplantation and with sufficient RNA for urinary cell RNA profiling. The kidney allograft recipients designated as patients with stable graft function did not undergo biopsy during the first 400 days of transplantation and met the following additional criteria: (i) average serum creatinine less than or equal to 2.0 mg per deciliter [180 micromol per liter] at 6, 9 and 12 months following transplantation, (ii) no treatment for acute rejection, and (iii) no evidence of cytomegalovirus (CMV) or polyomavirus type BK (BKV) infection.

The number of patients in the three categories listed under the metabolomics (FIG. 1) exceeded 241 unique patients because several patients had multiple urine samples and contributed urine samples to more than one category, that is the same patient contributing biopsy matched urine sample as well as sequential urine samples and being counted in both categories.

Two of the 1,518 samples were excluded from all analyses: one biopsy-matched urine sample (sample ID: 4202) from a patient (patient ID: 10137) with a No Rejection biopsy result was excluded due to failed osmolality measurements and only 80 metabolites being measured in that sample; and one of the sequential, non-biopsy associated samples from a patient with other biopsy findings (urine sample ID:0988; patient ID: 10014) did not contain sufficient cell free supernatant for non-targeted metabolite analysis. After exclusion of these two samples, high-quality metabolite data from 1516 samples collected from 241 kidney patients were available for data analysis.

Among the 298 biopsy matched urine samples, all 50 ACR biopsy matched urine samples from 36 patients and all 198 No Rejection biopsy urine samples from 149 patients were included in the analysis to determine whether metabolite profiles are diagnostic of acute cellular rejection. Urine samples matched to biopsies classified as borderline changes (27 samples from 25 patients), AMR (13 samples from 11 patients) and other findings (9 samples from 8 patients) were all also analyzed but excluded from the analyses to generate the diagnostic signature distinguishing ACR biopsies from No Rejection biopsies. The sequential samples preceding borderline changes, AMR and other findings were also not included in the analysis to determine whether the signature distinguishes patients who developed ACR from those whose biopsies did not show rejection changes (No Rejection biopsies).

A total of 2782 urine samples collected during the parent CTOT-04 study were excluded from non-targeted metabolite analysis because: (1) the urine samples were not matched to biopsy specimens or were collected following a biopsy (1074 specimens from 187 patients), (2) had unstable allograft function and no biopsy or were lost to follow up (272 urine samples from 63 patients) or (3) had stable graft function but had less than 10 sequential samples collected in the first 400 days of transplantation; urine samples collected after 400 days and with insufficient RNA for RNA profiling are also included in this group of 1436 specimens from 180 patients. The number of patients in the three categories excluded from metabolomics exceeds 243 unique patients because several patients had multiple urine samples and contributed urine samples to more than one category and thereby counted in each category.

### Metabolomics Data Acquisition.

The extracted samples were split into equal parts for analysis on the GC/MS and LC/MS/MS platforms; also included were several technical replicate samples created from a homogeneous pool containing a small amount of all study samples (“Client Matrix”). Instrument variability was determined by calculating the median relative standard deviation (RSD) for the internal standards that were added to each sample prior to injection into the mass spectrometers. Overall process variability was determined by calculating the median RSD for all endogenous metabolites (i.e., non-instrument standards) present in 100% of the Client Matrix samples, which are technical replicates of pooled client samples. Metabolites of known structural identity (named biochemicals) as well as metabolites of unknown structural identity (unnamed biochemicals) detected in the samples were detected.

Non-targeted metabolomics of urine was performed at Metabolon Inc. (Durham). Following receipt of samples by Metabolon, samples were inventoried and immediately stored at −80° C. Samples were maintained at −80° C. until processed. Sample preparation was carried out using an automated system (MicroLab STAR, Hamilton Robotics). Recovery standards were added prior to the first step in the extraction process for QC purposes. At the time of analysis, samples were extracted and prepared for analysis using Metabolon's standard solvent extraction method. The extracted samples were split into equal parts for analysis on the GC/MS and LC/MS/MS platforms. Sample preparation was conducted using a proprietary series of organic and aqueous extractions to remove the protein fraction while allowing maximum recovery of small molecules. The resulting extract was divided into two fractions; one for analysis by LC and one for analysis by GC.

The samples were placed briefly on a TurboVap (Zymark) to remove the organic solvent. Each sample was then frozen, dried under vacuum, and prepared for the appropriate instrument, either LC/MS or GC/MS. For QA/QC purposes, a number of additional samples were included with each day's analysis. Furthermore, a selection of QC metabolites was added to every sample, including those under test. These metabolites were carefully chosen so as not to interfere with the measurement of the endogenous metabolites.

### Liquid Chromatography/Mass Spectrometry (LC/MS, LC/MS2) for Metabolite Profiling.

The LC/MS portion of the platform was based on a Waters ACQUITY UPLC and a Thermo-Finnigan LTQ mass spectrometer, which consisted of an electrospray ionization (ESI) source and linear ion-trap (LIT) mass analyzer. The dried sample extract was split into two aliquots, and then reconstituted in acidic or basic LC-compatible solvents, each of which contained 11 or more injection standards at fixed concentrations. One aliquot was analyzed using acidic positive ion optimized conditions and the other using basic negative ion optimized conditions in two independent injections using separate dedicated columns (Waters UPLC BEH C18-2.1×100 mm, 1.7 μm). Extracts reconstituted in acidic conditions were gradient eluted using water and methanol both containing 0.1% formic acid, while the basic extracts, which also used water/methanol, contained 6.5 mM ammonium bicarbonate. The MS analysis alternated between MS and data-dependent MS2 scans, using dynamic exclusion, and the scan range was from 80-1000 m/z. For ions with counts greater than 2 million, an accurate mass measurement could be performed. Accurate mass measurements were made for the parent ion as well as fragments. The typical mass error was less than 5 ppm. Ions with less than two million counts require a greater amount of effort to characterize. Fragmentation spectra (MS/MS) were typically generated in a data dependent manner, but if necessary, targeted MS/MS was employed, such as in the case of lower level signals.

### Gas Chromatography/Mass Spectrometry (GC/MS) for Metabolite Profiling.

The samples destined for GC/MS analysis were re-dried under vacuum desiccation for a minimum of 24 hours prior to being derivatized under dried nitrogen using bistrimethyl-silyl-trifluoroacetamide (BSTFA). The GC column was 5% diphenyl/95% dimethyl polysiloxane fused silica column (20 m×0.18 mm ID; 0.18 urn film thickness) with helium as the carrier gas and a temperature ramp from 60° to 340° C. over a 17.5-min period. Samples were analyzed on a Thermo-Finnigan Trace DSQ fast-scanning single-quadruple mass spectrometer using electron impact ionization and operated at unit mass resolving power. The scan range was 50-750 m/z. The instrument was tuned and calibrated for mass resolution and mass accuracy on a daily basis.

### Metabolite Identification

The informatics system consisted of four major components, the Metabolon Laboratory Information Management System, the data extraction and peak-identification software, data processing tools for QC and metabolite identification, and a collection of information interpretation and visualization tools for use by data analysts. The data extraction of the raw mass spectroscopy data files yielded information that was loaded into a relational database. Once in the database the information was examined and appropriate QC limits were imposed, peaks were identified using Metabolon's proprietary peak integration software; and component parts were stored in a separate, specifically designed complex data structure.

Metabolites were identified by comparison to library entries of purified standards or recurrent unknown entities. More than 3,500 commercially available purified standard compounds have been acquired and registered into the LIMS for distribution to both the LC and GC platforms for determination of their analytical characteristics. The combination of chromatographic properties and mass spectra provided a match to a specific compound or an isobaric entity.

Using the Metabolon platform, metabolite data (ion-counts) was obtained for a total of 1516 urine samples. In total, 749 different metabolites from 65 metabolic pathways, including 368 metabolites of unknown identity, were identified on at least one of the three mass spectrometry based metabolomics platforms. A first data normalization step was performed to correct for variation resulting from instrument inter-day tuning differences. Essentially, each compound was corrected in run-day blocks by registering the medians to equal one and normalizing each data point proportionately. Values were then divided by urine osmolality and log₁₀-scaled. Outliers of more than four standard deviations (S.D.) from the mean were removed. Mean and S.D. values were determined on the 5% to 95% range of the data for this purpose. It was rare that more than two outlier values were removed from a metabolite. Finally, the data were z-scaled (mean=0, s.d.=1).

### Metabolomics Data.

In total, 749 different metabolites from 65 metabolic pathways, including 368 metabolites of unknown identity, were identified on at least one of the three mass spectrometry based metabolomics platforms. 358 metabolites were reported from the LC/MS platform running in negative ionization mode, 245 using LC/MS in positive mode, and 146 using GC/MS. The median number of different metabolites detected in any single sample was 616. The median technical error as determined from technical replicates on pooled sample material (s.d./mean) was 10.2%, while the median experimental variance computed from data on all patient samples was 113.9%, showing overall excellent signal-to-noise performance.

### Stability of Metabolites in Urine.

The stability of metabolites in stored samples is can be an issue; urine supernatants stored for different durations were not analyzed. The data obtained indicates that urine supernatants stored over several years yield robust metabolite signatures diagnostic of acute cellular rejection. The first urine specimen included the 1516 samples analyzed for metabolites was collected in 2006 and the last urine specimen was collected in 2009. Non-targeted metabolite analysis of the stored urine supernatants was performed in year 2012 at Metabolon Inc. and showed that the metabolite profiles including the ratio of 3-sialyllactose to xanthosine (3SL/X) are diagnostic of acute cellular rejection. Rapid Fire-QTOF MS/MS assay for the measurement of 3SL/X ratio was developed and the assays demonstrated that the ratio of 3SL/X is diagnostic of acute cellular rejection. Importantly, the observed correlation between the 3SL/X ratio calculated from the Metabolon data and the 3SL/X ratio measured using RapidFire-QTOF assay was 0.65 (Pearson R), and the Bland-Altman method for comparison showed that only 12 samples (6%) were beyond the 95% limit of agreement.

### Statistical Analyses

R and R-studio (R-project, versions 2.12.1 and 3.0.1) and SPSS (IBM, version 21) were used for statistical analysis. Linear models were computed using the lm-package, logistic models using the glm-package (family binomial), and ROC curves using the ROC-package. Linear regression [Mᵢ=f(ACR status)] and logistic regression [ACR=f(Mᵢ)] were computed for all 749 log-normalized and then z-scored metabolite data vectors (Mᵢ) for all samples with valid data taken at the time of a biopsy diagnosed as either ACR (coded 1) or “No rejection” (coded 0). Full association data from these analyses are provided in Tables 2 and 3.

For Table 2, the numerator refers to a Metabolon compound identifier in the numerator; the denominator refers to a Metabolon compound identifier in the denominator (where ‘one’ indicates ‘no ratio’); the term ncases refers to the number of data points used in the statistics; the term beta refers to the differences in mean levels of metabolites between ACR and No Rejection groups; the p-value refers to the p-value of the association with ACR; the p-gain refers to the p-gain of the association with ACR (when using ratios).

As shown, the difference in mean levels of metabolites quinolinate/X-16397 between ACR and No Rejection groups is 0.89. The difference in mean levels of metabolites 3-sialyllactose/xanthosine is 0.86.

TABLE 2
Linear regression [Mi = f(ACR)]
using all samples with matching biopsy

| numerator | denominator | ncases | beta | p-value | p-gain |
|---|---|---|---|---|---|
| quinolinate | X-16397 | 248 | 0.89 | 7.3E−09 | 25,031 |
| quinolinate | 4-hydroxy | 248 | 0.88 | 1.1E−08 | 16,954 |
| | mandelate | | | | |
| neopterin | xanthosine | 234 | 0.90 | 2.0E−08 | 72,220 |
| quinolinate | X-16570 | 242 | 0.87 | 2.6E−08 | 7,103 |
| proline | X-13723 | 233 | 0.87 | 2.8E−08 | 1,749 |
| quinolinate | xylitol | 247 | 0.86 | 3.1E−08 | 5,796 |
| quinolinate | X-12748 | 248 | 0.85 | 3.5E−08 | 5,259 |
| 3-sialyllactose | xanthosine | 242 | 0.86 | 5.0E−08 | 201,100 |
| X-12117 | X-13723 | 229 | 0.86 | 6.6E−08 | 5,568 |
| quinolinate | cis-aconitate | 246 | 0.83 | 9.0E−08 | 2,012 |
| quinolinate | ribulose | 241 | 0.85 | 9.2E−08 | 1,984 |
| neopterin | X-16570 | 235 | 0.84 | 9.7E−08 | 14,493 |
| neopterin | N1-methyl- | 238 | 0.83 | 1.2E−07 | 11,361 |
| | guanosine | | | | |
| quinolinate | homo- | 247 | 0.82 | 1.4E−07 | 1,273 |
| | vanillate | | | | |
| | (HVA) | | | | |
| quinolinate | X-13723 | 235 | 0.83 | 1.6E−07 | 1,155 |
| X-12690 | xanthosine | 242 | 0.83 | 1.6E−07 | 61,644 |
| X-12117 | xanthosine | 236 | 0.83 | 2.0E−07 | 2,374 |
| 3-sialyllactose | X-16397 | 247 | 0.77 | 5.9E−07 | 26,062 |
| fucitol | xylitol | 244 | 0.77 | 6.1E−07 | 4,288 |
| neopterin | X-12748 | 240 | 0.77 | 9.9E−07 | 1,433 |
| kynurenate | xanthosine | 241 | 0.78 | 1.0E−06 | 1,262 |
| neopterin | xylitol | 239 | 0.77 | 1.2E−06 | 1,165 |
| neopterin | X-16674 | 224 | 0.78 | 1.4E−06 | 1,002 |
| neopterin | N2-methyl- | 236 | 0.77 | 1.6E−06 | 910 |
| | guanosine | | | | |
| X-12690 | N2-methyl- | 242 | 0.76 | 1.6E−06 | 3,297 |
| | guanosine | | | | |
| fucitol | N2-methyl- | 239 | 0.76 | 1.6E−06 | 3,259 |
| | guanosine | | | | |
| X-17366 | N2-methyl- | 237 | 0.75 | 2.9E−06 | 1,830 |
| | guanosine | | | | |
| 3-sialyllactose | X-16570 | 242 | 0.73 | 3.1E−06 | 1,115 |
| fucitol | 4-hydroxy- | 244 | 0.72 | 3.7E−06 | 1,493 |
| | mandelate | | | | |
| X-17366 | xanthosine | 237 | 0.74 | 3.7E−06 | 2,732 |
| X-18486 | N2-methyl- | 243 | 0.74 | 3.9E−06 | 1,338 |
| | guanosine | | | | |
| N-acetyl- | N2-methyl- | 242 | 0.74 | 4.0E−06 | 1,325 |
| neuraminate | guanosine | | | | |
| X-18486 | xanthosine | 242 | 0.73 | 4.7E−06 | 2,172 |
| X-12715 | 4-hydroxy- | 247 | 0.71 | 5.1E−06 | 814 |
| | phenyl- | | | | |
| | acetate | | | | |
| hydantoin-5- | xanthosine | 223 | 0.74 | 5.4E−06 | 1,894 |
| propionic acid | | | | | |
| 5-oxoproline | xanthosine | 242 | 0.70 | 1.1E−05 | 898 |
| X-11945 | xanthosine | 242 | 0.70 | 1.3E−05 | 788 |
| X-11491 | 4-imidazole- | 132 | 1.07 | 1.4E−05 | 3,701 |
| | acetate | | | | |
| fucitol | isocitrate | 243 | 0.67 | 1.7E−05 | 3,699 |
| fucitol | ribitol | 243 | 0.66 | 2.3E−05 | 886 |
| X-11491 | 7-methyl- | 128 | 1.00 | 2.6E−05 | 1,977 |
| | xanthine | | | | |
| fucitol | xylulose | 244 | 0.64 | 4.5E−05 | 899 |
| proline | one | 245 | 0.63 | 4.9E−05 | 1 |
| fucitol | ribulose | 238 | 0.64 | 6.3E−05 | 835 |
| fucitol | 5-amino- | 233 | 0.64 | 7.4E−05 | 849 |
| | levulinate | | | | |
| X-11491 | X-12267 | 150 | 0.86 | 8.7E−05 | 1,547 |
| 3-hydroxy- | X-13724 | 165 | 0.77 | 9.1E−05 | 825 |
| butyrate | | | | | |
| (BHBA) | | | | | |
| diglycerol | X-12747 | 91 | 1.09 | 9.7E−05 | 947 |
| quinolinate | one | 247 | 0.59 | 1.8E−04 | 1 |
| X-12832 | X-11452 | 202 | 0.64 | 2.0E−04 | 811 |
| isoleucine | one | 244 | 0.58 | 2.1E−04 | 1 |
| X-13723 | one | 235 | −0.57 | 3.7E−04 | 1 |
| X-12117 | one | 242 | 0.55 | 4.9E−04 | 1 |
| X-16940 | one | 241 | −0.55 | 5.8E−04 | 1 |
| leucine | one | 246 | 0.52 | 9.9E−04 | 1 |
| pipecolate | one | 239 | 0.52 | 1.1E−03 | 1 |
| paraxanthine | one | 158 | −0.63 | 1.1E−03 | 1 |
| 1,5-anhydro- | one | 197 | 0.57 | 1.1E−03 | 1 |
| glucitol | | | | | |
| (1,5-AG) | | | | | |
| X-19434 | one | 126 | 0.77 | 1.3E−03 | 1 |
| kynurenate | one | 245 | 0.51 | 1.3E−03 | 1 |
| neopterin | one | 240 | 0.51 | 1.4E−03 | 1 |
| myo-inositol | one | 248 | 0.49 | 1.7E−03 | 1 |
| gentisate | one | 243 | −0.50 | 1.8E−03 | 1 |
| valine | one | 243 | 0.49 | 1.9E−03 | 1 |
| N-methyl- | one | 175 | 0.58 | 2.5E−03 | 1 |
| acetaminophen | | | | | |
| sulfate 1* | | | | | |
| xylitol | one | 248 | −0.47 | 2.6E−03 | 1 |
| homovanillate | one | 247 | −0.47 | 3.1E−03 | 1 |
| (HVA) | | | | | |
| uracil | one | 248 | −0.47 | 3.1E−03 | 1 |
| trigonelline | one | 247 | −0.46 | 3.3E−03 | 1 |
| (N′-methyl- | | | | | |
| nicotinate) | | | | | |
| X-16570 | one | 242 | −0.47 | 3.5E−03 | 1 |
| 4-hydroxy- | one | 248 | −0.45 | 4.2E−03 | 1 |
| phenylacetate | | | | | |
| phenylalanine | one | 245 | 0.45 | 4.2E−03 | 1 |
| X-12230 | one | 212 | −0.50 | 4.6E−03 | 1 |
| N2-methyl- | one | 244 | −0.45 | 5.3E−03 | 1 |
| guanosine | | | | | |
| 4-hydroxy- | one | 248 | −0.44 | 5.5E−03 | 1 |
| mandelate | | | | | |
| xanthine | one | 247 | −0.43 | 5.8E−03 | 1 |
| 3-(N-acetyl-L- | one | 147 | 0.55 | 5.9E−03 | 1 |
| cystein-S-yl) | | | | | |
| acetaminophen* | | | | | |
| kynurenine | one | 245 | 0.43 | 6.4E−03 | 1 |
| phosphate | one | 248 | 0.43 | 6.6E−03 | 1 |
| X-14625 | one | 239 | 0.42 | 8.9E−03 | 1 |
| stachydrine | one | 248 | 0.41 | 9.3E−03 | 1 |
| X-17320 | one | 154 | −0.51 | 9.9E−03 | 1 |
| acetylcarnitine | one | 248 | 0.41 | 9.9E−03 | 1 |
| xanthosine | one | 242 | −0.42 | 1.0E−02 | 1 |
| X-17366 | one | 240 | 0.40 | 1.2E−02 | 1 |
| 2-methoxy- | one | 124 | 0.53 | 1.2E−02 | 1 |
| acetaminophen | | | | | |
| sulfate* | | | | | |
| X-17185 | one | 194 | −0.48 | 1.4E−02 | 1 |
| 4-vinylphenol | one | 239 | −0.39 | 1.4E−02 | 1 |
| sulfate | | | | | |
| X-12114 | one | 247 | 0.39 | 1.5E−02 | 1 |
| 3-sialyllactose | one | 248 | 0.38 | 1.5E−02 | 1 |
| X-17370 | one | 161 | 0.48 | 1.6E−02 | 1 |
| X-12221 | one | 234 | −0.39 | 1.6E−02 | 1 |
| urate | one | 248 | 0.38 | 1.6E−02 | 1 |
| lysine | one | 246 | 0.38 | 1.7E−02 | 1 |
| 3-hydroxy- | one | 162 | 0.43 | 1.7E−02 | 1 |
| kynurenine | | | | | |
| p-acetamido- | one | 170 | 0.46 | 1.7E−02 | 1 |
| phenyl- | | | | | |
| glucuronide | | | | | |
| hydantoin-5- | one | 229 | 0.38 | 1.7E−02 | 1 |
| propionic acid | | | | | |
| 1-(3-amino- | one | 235 | 0.38 | 1.7E−02 | 1 |
| propyl)-2- | | | | | |
| pyrrolidone | | | | | |
| citrate | one | 248 | −0.37 | 1.8E−02 | 1 |
| X-17735 | one | 232 | −0.39 | 1.8E−02 | 1 |
| 1-methyl- | one | 222 | −0.41 | 1.8E−02 | 1 |
| xanthine | | | | | |
| X-18945 | one | 232 | 0.38 | 1.8E−02 | 1 |
| 3-(3-hydroxy- | one | 170 | −0.45 | 1.9E−02 | 1 |
| phenyl)propionate | | | | | |
| arginine | one | 229 | 0.39 | 1.9E−02 | 1 |
| propionyl- | one | 233 | 0.37 | 1.9E−02 | 1 |
| carnitine | | | | | |
| 5-hydroxy- | one | 238 | −0.38 | 2.0E−02 | 1 |
| methyl-2-furoic | | | | | |
| acid | | | | | |
| ribitol | one | 247 | −0.37 | 2.1E−02 | 1 |
| X-12690 | one | 247 | 0.37 | 2.1E−02 | 1 |
| 3,4-dihydroxy- | one | 244 | −0.37 | 2.1E−02 | 1 |
| phenylacetate | | | | | |
| X-18887 | one | 246 | 0.36 | 2.1E−02 | 1 |
| 2-methyl- | one | 239 | 0.37 | 2.2E−02 | 1 |
| butyryl-carnitine | | | | | |
| (C5) | | | | | |
| imidazole | one | 245 | −0.36 | 2.3E−02 | 1 |
| lactate | | | | | |
| X-16947 | one | 245 | 0.36 | 2.3E−02 | 1 |
| X-11843 | one | 161 | 0.45 | 2.4E−02 | 1 |
| 3,5-dihydroxy- | one | 233 | −0.37 | 2.4E−02 | 1 |
| benzoic acid | | | | | |
| glucose | one | 248 | 0.35 | 2.5E−02 | 1 |
| X-15503 | one | 248 | 0.35 | 2.6E−02 | 1 |
| X-16674 | one | 232 | −0.36 | 2.6E−02 | 1 |
| pantothenate | one | 244 | −0.35 | 2.8E−02 | 1 |
| X-12379 | one | 205 | 0.38 | 2.9E−02 | 1 |
| X-12738 | one | 155 | −0.44 | 2.9E−02 | 1 |
| 1,7- | one | 223 | −0.37 | 2.9E−02 | 1 |
| dimethylurate | | | | | |
| X-17369 | one | 93 | 0.55 | 2.9E−02 | 1 |
| 3- | one | 248 | −0.34 | 3.1E−02 | 1 |
| aminoisobutyrate | | | | | |
| X-12306 | one | 133 | 0.61 | 3.1E−02 | 1 |
| catechol sulfate | one | 247 | −0.34 | 3.1E−02 | 1 |
| X-18486 | one | 248 | 0.34 | 3.1E−02 | 1 |
| 5alpha- | one | 97 | 0.55 | 3.2E−02 | 1 |
| androstan- | | | | | |
| 3beta,17beta-diol | | | | | |
| disulfate | | | | | |
| X-12107 | one | 229 | −0.37 | 3.3E−02 | 1 |
| X-12039 | one | 246 | −0.34 | 3.3E−02 | 1 |
| X-12685 | one | 225 | −0.35 | 3.3E−02 | 1 |
| androsterone | one | 233 | 0.34 | 3.4E−02 | 1 |
| sulfate | | | | | |
| X-12364 | one | 228 | 0.36 | 3.4E−02 | 1 |
| homocitrulline | one | 244 | 0.34 | 3.4E−02 | 1 |
| X-12840 | one | 183 | 0.39 | 3.5E−02 | 1 |
| 3-(cystein-S-yl)- | one | 133 | 0.47 | 3.5E−02 | 1 |
| acetaminophen* | | | | | |
| X-17398 | one | 178 | −0.40 | 3.6E−02 | 1 |
| 1,6- | one | 248 | 0.33 | 3.6E−02 | 1 |
| anhydroglucose | | | | | |
| X-13726 | one | 240 | 0.34 | 3.7E−02 | 1 |
| 4-acetaminophen | one | 203 | 0.36 | 3.9E−02 | 1 |
| sulfate | | | | | |
| hypoxanthine | one | 246 | −0.33 | 3.9E−02 | 1 |
| X-17350 | one | 211 | 0.35 | 3.9E−02 | 1 |
| azelate | one | 220 | −0.34 | 4.1E−02 | 1 |
| (nonanedioate) | | | | | |
| xylulose | one | 247 | −0.32 | 4.1E−02 | 1 |
| cystine | one | 188 | 0.37 | 4.2E−02 | 1 |
| methionine | one | 246 | 0.32 | 4.2E−02 | 1 |
| X-17300 | one | 130 | −0.45 | 4.4E−02 | 1 |
| X-12125 | one | 247 | 0.31 | 4.8E−02 | 1 |
| X-12855 | one | 211 | 0.34 | 4.8E−02 | 1 |

For Table 3, the numerator refers to a Metabolon compound identifier in the numerator; the denominator refers to a Metabolon compound identifier in the denominator (where ‘one’ indicates ‘no ratio’); the term ncases refers to the number of data points used in the statistics; the term OR refers to the odds ratio of the association with ACR, per 1 standard deviation change in predictor; the p-value refers to the p-value of the association with ACR; and the p-gain refers to the p-gain of the association with ACR (when using ratios).

TABLE 3
Logistic regression [ACR = f(Mi)] using
all samples with matching biopsy

| Numerator | denominator | ncases | OR | p-value | p-gain |
|---|---|---|---|---|---|
| Quinolinate | X-16397 | 248 | 2.59 | 1.9E−07 | 1,769 |
| neopterin | xanthosine | 234 | 2.54 | 6.0E−07 | 3,658 |
| 3-sialyllactose | xanthosine | 242 | 2.47 | 9.9E−07 | 11,463 |
| neopterin | X-16570 | 235 | 2.46 | 1.3E−06 | 1,642 |
| X-12690 | xanthosine | 242 | 2.54 | 1.7E−06 | 6,590 |
| neopterin | N1-methyl- | 238 | 2.36 | 1.9E−06 | 1,126 |
| | guanosine | | | | |
| fucitol | xylitol | 244 | 2.33 | 4.1E−06 | 833 |
| 3-sialyllactose | X-16397 | 247 | 2.24 | 4.5E−06 | 3,778 |
| fucitol | isocitrate | 243 | 2.02 | 4.9E−05 | 1,324 |
| proline | one | 245 | 1.79 | 1.4E−04 | 1 |
| quinolinate | one | 247 | 1.81 | 3.4E−04 | 1 |
| isoleucine | one | 244 | 1.77 | 4.5E−04 | 1 |
| X-13723 | one | 235 | 0.52 | 6.1E−04 | 1 |
| X-12117 | one | 242 | 1.70 | 8.5E−04 | 1 |
| X-16940 | one | 241 | 0.57 | 9.0E−04 | 1 |
| pipecolate | one | 239 | 1.63 | 1.6E−03 | 1 |
| leucine | one | 246 | 1.62 | 1.7E−03 | 1 |
| kynurenate | one | 245 | 1.63 | 2.0E−03 | 1 |
| neopterin | one | 240 | 1.61 | 2.2E−03 | 1 |
| myo-inositol | one | 248 | 1.70 | 2.3E−03 | 1 |
| gentisate | one | 243 | 0.59 | 2.4E−03 | 1 |
| valine | one | 243 | 1.63 | 2.5E−03 | 1 |
| xylitol | one | 248 | 0.62 | 3.4E−03 | 1 |
| uracil | one | 248 | 0.62 | 3.9E−03 | 1 |
| trigonelline (N′- | one | 247 | 0.62 | 4.2E−03 | 1 |
| methylnicotinate) | | | | | |
| X-16570 | one | 242 | 0.62 | 4.3E−03 | 1 |
| homovanillate | one | 247 | 0.64 | 4.7E−03 | 1 |
| (HVA) | | | | | |
| 4-hydroxy | one | 248 | 0.62 | 5.1E−03 | 1 |
| phenylacetate | | | | | |
| phenyl- | one | 245 | 1.55 | 5.3E−03 | 1 |
| alanine | | | | | |
| X-12230 | one | 212 | 0.59 | 5.7E−03 | 1 |
| N2-methyl- | one | 244 | 0.63 | 6.2E−03 | 1 |
| guanosine | | | | | |
| 4-hydroxy- | one | 248 | 0.64 | 6.6E−03 | 1 |
| mandelate | | | | | |
| xanthine | one | 247 | 0.64 | 6.7E−03 | 1 |
| phosphate | one | 248 | 1.64 | 7.2E−03 | 1 |
| kynurenine | one | 245 | 1.56 | 7.5E−03 | 1 |
| X-14625 | one | 239 | 1.52 | 1.0E−02 | 1 |
| stachydrine | one | 248 | 1.54 | 1.0E−02 | 1 |
| xanthosine | one | 242 | 0.65 | 1.1E−02 | 1 |
| acetylcarnitine | one | 248 | 1.51 | 1.1E−02 | 1 |
| X-17366 | one | 240 | 1.49 | 1.4E−02 | 1 |
| 4-vinylphenol | one | 239 | 0.67 | 1.5E−02 | 1 |
| sulfate | | | | | |
| X-12114 | one | 247 | 1.49 | 1.6E−02 | 1 |
| 3-sialyllactose | one | 248 | 1.44 | 1.7E−02 | 1 |
| X-12221 | one | 234 | 0.67 | 1.7E−02 | 1 |
| urate | one | 248 | 1.49 | 1.8E−02 | 1 |
| lysine | one | 246 | 1.46 | 1.8E−02 | 1 |
| hydantoin-5- | one | 229 | 1.44 | 1.9E−02 | 1 |
| propionic acid | | | | | |
| 1-(3-aminopropyl)- | one | 235 | 1.45 | 1.9E−02 | 1 |
| 2-pyrrolidone | | | | | |
| citrate | one | 248 | 0.69 | 2.0E−02 | 1 |
| X-17735 | one | 232 | 0.66 | 2.0E−02 | 1 |
| 1-methylxanthine | one | 222 | 0.65 | 2.0E−02 | 1 |
| X-18945 | one | 232 | 1.47 | 2.0E−02 | 1 |
| arginine | one | 229 | 1.46 | 2.1E−02 | 1 |
| propionyl- | one | 233 | 1.47 | 2.1E−02 | 1 |
| carnitine | | | | | |
| 5-hydroxymethyl- | one | 238 | 0.67 | 2.1E−02 | 1 |
| 2-furoic acid | | | | | |
| X-18887 | one | 246 | 1.46 | 2.2E−02 | 1 |
| ribitol | one | 247 | 0.70 | 2.3E−02 | 1 |
| X-12690 | one | 247 | 1.44 | 2.3E−02 | 1 |
| 3,4-dihydroxy- | one | 244 | 0.70 | 2.4E−02 | 1 |
| phenylacetate | | | | | |
| 2-methylbutyryl- | one | 239 | 1.48 | 2.4E−02 | 1 |
| carnitine (C5) | | | | | |
| imidazole lactate | one | 245 | 0.69 | 2.4E−02 | 1 |
| X-16947 | one | 245 | 1.43 | 2.5E−02 | 1 |
| 3,5-dihydroxy- | one | 233 | 0.66 | 2.6E−02 | 1 |
| benzoic acid | | | | | |
| X-15503 | one | 248 | 1.43 | 2.7E−02 | 1 |
| glucose | one | 248 | 1.40 | 2.7E−02 | 1 |
| X-16674 | one | 232 | 0.68 | 2.7E−02 | 1 |
| pantothenate | one | 244 | 0.70 | 2.9E−02 | 1 |
| 1,7-dimethylurate | one | 223 | 0.70 | 3.1E−02 | 1 |
| X-12379 | one | 205 | 1.45 | 3.1E−02 | 1 |
| 3-aminoisobutyrate | one | 248 | 0.70 | 3.2E−02 | 1 |
| X-18486 | one | 248 | 1.44 | 3.3E−02 | 1 |
| catechol sulfate | one | 247 | 0.72 | 3.4E−02 | 1 |
| X-12107 | one | 229 | 0.67 | 3.4E−02 | 1 |
| X-12039 | one | 246 | 0.71 | 3.5E−02 | 1 |
| X-12685 | one | 225 | 0.68 | 3.5E−02 | 1 |
| X-12364 | one | 228 | 1.46 | 3.5E−02 | 1 |
| androsterone | one | 233 | 1.42 | 3.6E−02 | 1 |
| sulfate | | | | | |
| homocitrulline | one | 244 | 1.40 | 3.6E−02 | 1 |
| 1,6-anhydro- | one | 248 | 1.40 | 3.8E−02 | 1 |
| glucose | | | | | |
| X-13726 | one | 240 | 1.42 | 3.9E−02 | 1 |
| hypoxanthine | one | 246 | 0.71 | 4.0E−02 | 1 |
| 4-acetaminophen | one | 203 | 1.44 | 4.1E−02 | 1 |
| sulfate | | | | | |
| X-17350 | one | 211 | 1.41 | 4.1E−02 | 1 |
| azelate | one | 220 | 0.69 | 4.3E−02 | 1 |
| (nonanedioate) | | | | | |
| xylulose | one | 247 | 0.73 | 4.3E−02 | 1 |
| methionine | one | 246 | 1.36 | 4.5E−02 | 1 |
| X-12125 | one | 247 | 1.36 | 4.9E−02 | 1 |

Similarly, linear and logistic regressions were also computed limiting the sample set further to samples with both metabolite data and successful RNA quantification, that is, samples with available RNA signatures. Full association data from these analyses are provided in Tables 4, 5, and 6.

For Table 4, the numerator refers to a Metabolon compound identifier in the numerator; the denominator refers to a Metabolon compound identifier in the denominator (where ‘one’ indicates ‘no ratio’); the term ncases refers to the number of data points used in the statistics; the term beta refers to the differences in mean levels of metabolites between ACR and No Rejection groups; the p-value refers to the p-value of the association with ACR; the p-gain refers to the p-gain of the association with ACR (when using ratios).

TABLE 4
Linear regression [Mi = f(ACR)] using all samples
with matching biopsy and available RNA signature

| numerator | denominator | ncases | beta | p-value | p-gain |
|---|---|---|---|---|---|
| quinolinate | 4-hydroxy- | 206 | 0.96 | 6.7E−09 | 59,573 |
| | mandelate | | | | |
| quinolinate | xylitol | 205 | 0.93 | 2.3E−08 | 17,578 |
| 3- | xanthosine | 201 | 0.93 | 4.5E−08 | 191,212 |
| sialyllactose | | | | | |
| proline | 4-hydroxy- | 205 | 0.90 | 5.1E−08 | 823 |
| | phenylacetate | | | | |
| quinolinate | X-16397 | 206 | 0.89 | 8.2E−08 | 4,854 |
| neopterin | xanthosine | 195 | 0.92 | 1.0E−07 | 50,721 |
| quinolinate | xylulose | 206 | 0.86 | 2.2E−07 | 1,773 |
| quinolinate | Homovanillate | 205 | 0.87 | 2.3E−07 | 1,692 |
| | (HVA) | | | | |
| pipecolate | 4-hydroxy- | 199 | 0.87 | 2.4E−07 | 1,763 |
| | phenylacetate | | | | |
| X-12117 | xanthosine | 196 | 0.89 | 2.7E−07 | 2,971 |
| neopterin | N1-methyl- | 198 | 0.87 | 3.0E−07 | 17,759 |
| | guanosine | | | | |
| quinolinate | arabitol | 206 | 0.85 | 3.4E−07 | 1,158 |
| X-17366 | xanthosine | 197 | 0.88 | 3.5E−07 | 24,658 |
| X-12117 | X-13723 | 193 | 0.87 | 4.0E−07 | 2,048 |
| X-18486 | xanthosine | 201 | 0.87 | 4.7E−07 | 18,221 |
| quinolinate | X-16570 | 203 | 0.85 | 4.9E−07 | 802 |
| neopterin | 4-hydroxy- | 200 | 0.85 | 5.3E−07 | 796 |
| | phenylacetate | | | | |
| pipecolate | xanthosine | 194 | 0.87 | 6.2E−07 | 912 |
| X-12117 | xylonate | 201 | 0.84 | 6.3E−07 | 1,292 |
| kynurenate | xanthosine | 201 | 0.85 | 7.1E−07 | 12,163 |
| X-12117 | galactose | 200 | 0.84 | 7.2E−07 | 1,126 |
| X-12114 | 4-hydroxy- | 204 | 0.82 | 8.2E−07 | 1,364 |
| | mandelate | | | | |
| X-12117 | N2-methyl- | 195 | 0.86 | 8.4E−07 | 961 |
| | guanosine | | | | |
| neopterin | xylitol | 199 | 0.84 | 8.4E−07 | 816 |
| X-12117 | 4-hydroxy- | 201 | 0.83 | 8.8E−07 | 917 |
| | mandelate | | | | |
| X-12117 | aconate | 198 | 0.83 | 1.1E−06 | 751 |
| | (methyl- | | | | |
| | fumarate) | | | | |
| 2-methyl- | xanthine | 199 | 0.82 | 1.3E−06 | 865 |
| butyryl- | | | | | |
| carnitine | | | | | |
| (C5) | | | | | |
| X-11491 | X-13723 | 126 | 1.09 | 1.3E−06 | 929 |
| neopterin | X-16570 | 197 | 0.82 | 1.5E−06 | 3,384 |
| X-12690 | xanthosine | 201 | 0.82 | 1.8E−06 | 4,760 |
| neopterin | X-16674 | 186 | 0.83 | 2.0E−06 | 2,612 |
| 3-sialyl- | X-16397 | 206 | 0.79 | 2.3E−06 | 11,237 |
| lactose | | | | | |
| methionine | xanthosine | 199 | 0.82 | 2.4E−06 | 1,764 |
| X-17366 | N2-methyl- | 197 | 0.82 | 2.4E−06 | 3,487 |
| | guanosine | | | | |
| neopterin | aconate | 197 | 0.80 | 2.7E−06 | 1,928 |
| | (methyl- | | | | |
| | fumarate) | | | | |
| stachydrine | elline | 206 | 0.78 | 2.8E−06 | 770 |
| | (N′-methyl- | | | | |
| | nicot | | | | |
| X-11491 | 4-imidazole- | 114 | 1.21 | 2.8E−06 | 16,735 |
| | acetate | | | | |
| neopterin | X-12748 | 200 | 0.79 | 3.5E−06 | 1,514 |
| X-18486 | N2-methyl- | 201 | 0.80 | 4.1E−06 | 2,026 |
| | guanosine | | | | |
| neopterin | trans-urocanate | 199 | 0.78 | 4.1E−06 | 1,268 |
| neopterin | hylarginine | 200 | 0.77 | 5.2E−06 | 1,005 |
| | (SDMA + A | | | | |
| X-11491 | X-12267 | 126 | 1.02 | 1.0E−05 | 4,717 |
| 3-sialyl- | X-12748 | 205 | 0.73 | 1.2E−05 | 2,059 |
| lactose | | | | | |
| X-11491 | 7-methyl- | 109 | 1.05 | 1.6E−05 | 2,946 |
| | xanthine | | | | |
| glucose | melate | 185 | 0.77 | 1.7E−05 | 784 |
| | (heptanedioate) | | | | |
| 3-sialyl- | aconate | 203 | 0.71 | 2.4E−05 | 1,055 |
| lactose | (methyl- | | | | |
| | fumarate) | | | | |
| 3-sialyl- | N1-methyl- | 205 | 0.71 | 2.6E−05 | 985 |
| lactose | guanosine | | | | |
| 5-oxoproline | aconate | 203 | 0.71 | 2.8E−05 | 1,303 |
| | (methyl- | | | | |
| | fumarate) | | | | |
| pregnendiol | Droxy- | 163 | 0.83 | 3.5E−05 | 3,508 |
| disulfate* | pregnenolone | | | | |
| | disulfate | | | | |
| proline | one | 204 | 0.69 | 4.2E−05 | 1 |
| leucine | one | 203 | 0.69 | 4.4E−05 | 1 |
| X-11491 | X-12712 | 128 | 0.94 | 5.1E−05 | 919 |
| X-11491 | X-17349 | 66 | 1.16 | 5.2E−05 | 899 |
| X-11491 | 1-methyl- | 120 | 1.00 | 5.4E−05 | 868 |
| | xanthine | | | | |
| X-12637 | X-17699 | 165 | 0.79 | 5.5E−05 | 994 |
| 1,5-anhydro- | one | 165 | 0.74 | 7.0E−05 | 1 |
| glucitol | | | | | |
| (1,5-AG) | | | | | |
| X-17327 | X-12819 | 201 | 0.66 | 1.1E−04 | 1,256 |
| X-12116 | galactose | 184 | 0.70 | 1.4E−04 | 882 |
| 3-hydroxy- | X-13724 | 136 | 0.80 | 1.6E−04 | 759 |
| butyrate | | | | | |
| (BHBA) | | | | | |
| X-12742 | X-12819 | 202 | 0.64 | 1.8E−04 | 771 |
| fucitol | allo-threonine | 203 | 0.63 | 2.1E−04 | 935 |
| isoleucine | one | 202 | 0.62 | 2.8E−04 | 1 |
| N-methyl | one | 145 | 0.74 | 3.4E−04 | 1 |
| Acetaminophen | | | | | |
| sulfate 1* | | | | | |
| fucitol | arabitol | 204 | 0.61 | 3.5E−04 | 779 |
| quinolinate | one | 205 | 0.60 | 4.0E−04 | 1 |
| 4-hydroxy- | one | 206 | −0.60 | 4.3E−04 | 1 |
| phenylacetate | | | | | |
| X-16940 | one | 201 | −0.60 | 5.5E−04 | 1 |
| pipecolate | one | 199 | 0.59 | 5.6E−04 | 1 |
| xylitol | one | 206 | −0.58 | 6.9E−04 | 1 |
| X-12117 | one | 201 | 0.58 | 8.1E−04 | 1 |
| Homo- | one | 205 | −0.57 | 8.2E−04 | 1 |
| vanillate | | | | | |
| (HVA) | | | | | |
| 3-(N-acetyl-L- | one | 119 | 0.71 | 1.1E−03 | 1 |
| cysteine-S-yl) | | | | | |
| acetamin- | | | | | |
| ophen* | | | | | |
| gentisate | one | 201 | −0.57 | 1.1E−03 | 1 |
| 4-hydroxy- | one | 205 | −0.55 | 1.1E−03 | 1 |
| mandelate | | | | | |
| xanthine | one | 206 | −0.55 | 1.1E−03 | 1 |
| X-13723 | one | 198 | −0.56 | 1.3E−03 | 1 |
| uracil | one | 206 | −0.55 | 1.3E−03 | 1 |
| phenylalanine | one | 204 | 0.53 | 1.8E−03 | 1 |
| stachydrine | one | 206 | 0.52 | 2.2E−03 | 1 |
| valine | one | 203 | 0.52 | 2.4E−03 | 1 |
| trigonelline | one | 206 | −0.51 | 2.5E−03 | 1 |
| (N′-methyl- | | | | | |
| nicotinate) | | | | | |
| X-12855 | one | 174 | 0.56 | 2.8E−03 | 1 |
| lysine | one | 204 | 0.51 | 2.8E−03 | 1 |
| X-17320 | one | 128 | −0.63 | 3.1E−03 | 1 |
| paraxanthine | one | 131 | −0.62 | 3.3E−03 | 1 |
| X-12230 | one | 177 | −0.56 | 3.4E−03 | 1 |
| 3,4- | one | 203 | −0.50 | 3.7E−03 | 1 |
| dihydroxy- | | | | | |
| phenylacetate | | | | | |
| 2-methoxy- | one | 101 | 0.67 | 3.8E−03 | 1 |
| Acetaminophen | | | | | |
| sulfate* | | | | | |
| methionine | one | 204 | 0.49 | 4.2E−03 | 1 |
| kynurenine | one | 204 | 0.48 | 4.8E−03 | 1 |
| X-16570 | one | 203 | −0.48 | 5.1E−03 | 1 |
| X-19434 | one | 110 | 0.71 | 5.1E−03 | 1 |
| neopterin | one | 200 | 0.48 | 5.2E−03 | 1 |
| X-12221 | one | 195 | −0.48 | 6.0E−03 | 1 |
| p-acetamido- | one | 137 | 0.55 | 7.8E−03 | 1 |
| phenyl- | | | | | |
| glucuronide | | | | | |
| 5-hydroxy- | one | 199 | −0.46 | 8.2E−03 | 1 |
| methyl- | | | | | |
| 2-furoic acid | | | | | |
| N2-methyl- | one | 202 | −0.46 | 8.4E−03 | 1 |
| guanosine | | | | | |
| acetyl- | one | 206 | 0.45 | 8.6E−03 | 1 |
| carnitine | | | | | |
| xanthosine | one | 201 | −0.46 | 8.6E−03 | 1 |
| kynurenate | one | 204 | 0.45 | 9.0E−03 | 1 |
| X-17366 | one | 200 | 0.45 | 9.3E−03 | 1 |
| ornithine | one | 204 | 0.44 | 9.4E−03 | 1 |
| X-14625 | one | 200 | 0.45 | 9.6E−03 | 1 |
| propionyl- | one | 193 | 0.45 | 1.0E−02 | 1 |
| carnitine | | | | | |
| 2-methyl- | one | 198 | 0.45 | 1.0E−02 | 1 |
| Butyryl- | | | | | |
| carnitine (C5) | | | | | |
| isobutyryl- | one | 200 | −0.45 | 1.1E−02 | 1 |
| glycine | | | | | |
| myoinositol | one | 206 | 0.43 | 1.2E−02 | 1 |
| imidazole | one | 203 | −0.43 | 1.2E−02 | 1 |
| lactate | | | | | |
| isovaleryl- | one | 203 | −0.43 | 1.3E−02 | 1 |
| glycine | | | | | |
| urate | one | 206 | 0.42 | 1.3E−02 | 1 |
| glucose | one | 206 | 0.42 | 1.3E−02 | 1 |
| mannose | one | 206 | 0.42 | 1.4E−02 | 1 |
| 5alpha- | one | 83 | 0.68 | 1.4E−02 | 1 |
| androstan- | | | | | |
| 3beta,17- | | | | | |
| beta-diol | | | | | |
| disulfate | | | | | |
| 3-hydroxy- | one | 133 | 0.48 | 1.5E−02 | 1 |
| kynurenine | | | | | |
| X-12039 | one | 205 | −0.42 | 1.5E−02 | 1 |
| isocitrate | one | 206 | −0.41 | 1.6E−02 | 1 |
| X-12107 | one | 187 | −0.46 | 1.6E−02 | 1 |
| X-11843 | one | 130 | 0.53 | 1.6E−02 | 1 |
| ribitol | one | 205 | −0.41 | 1.6E−02 | 1 |
| arginine | one | 191 | 0.42 | 1.8E−02 | 1 |
| 4-hydroxy- | one | 206 | −0.40 | 1.8E−02 | 1 |
| benzoate | | | | | |
| 3-(cystein- | one | 106 | 0.57 | 1.8E−02 | 1 |
| S-yl) | | | | | |
| Acetamin- | | | | | |
| ophen* | | | | | |
| X-17369 | one | 74 | 0.65 | 2.0E−02 | 1 |
| X-12685 | one | 187 | −0.42 | 2.0E−02 | 1 |
| X-17735 | one | 194 | −0.42 | 2.0E−02 | 1 |
| 3-amino- | one | 206 | −0.40 | 2.0E−02 | 1 |
| isobutyrate | | | | | |
| X-12193 | one | 204 | −0.40 | 2.1E−02 | 1 |
| X-16674 | one | 192 | −0.41 | 2.1E−02 | 1 |
| allantoin | one | 197 | 0.40 | 2.2E−02 | 1 |
| X-17185 | one | 161 | −0.49 | 2.2E−02 | 1 |
| X-12114 | one | 204 | 0.39 | 2.3E−02 | 1 |
| hydantoin- | one | 190 | 0.40 | 2.3E−02 | 1 |
| 5-propionic | | | | | |
| acid | | | | | |
| xylulose | one | 205 | −0.39 | 2.4E−02 | 1 |
| lactate | one | 202 | 0.39 | 2.4E−02 | 1 |
| 1,7-dimethyl- | one | 187 | −0.41 | 2.5E−02 | 1 |
| urate | | | | | |
| X-12849 | one | 206 | −0.38 | 2.5E−02 | 1 |
| trans- | one | 204 | −0.38 | 2.5E−02 | 1 |
| urocanate | | | | | |
| tyrosine | one | 204 | 0.38 | 2.5E−02 | 1 |
| 3-sialyl- | one | 206 | 0.38 | 2.6E−02 | 1 |
| lactose | | | | | |
| 3-(3-hydroxy- | one | 141 | −0.46 | 2.6E−02 | 1 |
| phenyl) | | | | | |
| propionate | | | | | |
| X-15461 | one | 200 | −0.39 | 2.6E−02 | 1 |
| citrate | one | 206 | −0.38 | 2.6E−02 | 1 |
| cystine | one | 164 | 0.42 | 2.8E−02 | 1 |
| pantothenate | one | 203 | −0.38 | 2.8E−02 | 1 |
| X-18965 | one | 201 | −0.38 | 2.9E−02 | 1 |
| X-12726 | one | 204 | −0.38 | 3.1E−02 | 1 |
| hypoxanthine | one | 206 | −0.37 | 3.2E−02 | 1 |
| X-17335 | one | 205 | −0.37 | 3.3E−02 | 1 |
| X-18486 | one | 206 | 0.36 | 3.4E−02 | 1 |
| 3,5-dihydroxy- | one | 194 | −0.38 | 3.4E−02 | 1 |
| benzoic acid | | | | | |
| X-15484 | one | 142 | 0.43 | 3.4E−02 | 1 |
| X-18887 | one | 205 | 0.36 | 3.5E−02 | 1 |
| X-11668 | one | 199 | −0.37 | 3.5E−02 | 1 |
| X-13850 | one | 139 | 0.43 | 3.6E−02 | 1 |
| N-(2-furoyl)- | one | 206 | −0.36 | 3.6E−02 | 1 |
| glycine | | | | | |
| guanine | one | 198 | −0.37 | 3.6E−02 | 1 |
| mesaconate | one | 202 | −0.36 | 3.7E−02 | 1 |
| (methyl- | | | | | |
| fumarate) | | | | | |
| glutamine | one | 204 | 0.36 | 3.7E−02 | 1 |
| X-17361 | one | 192 | −0.37 | 3.8E−02 | 1 |
| taurine | one | 193 | −0.38 | 4.2E−02 | 1 |
| X-17398 | one | 153 | −0.42 | 4.2E−02 | 1 |
| X-12747 | one | 132 | −0.43 | 4.4E−02 | 1 |
| 4-acetamino- | one | 169 | 0.38 | 4.5E−02 | 1 |
| phenyl sulfate | | | | | |
| X-12738 | one | 128 | −0.46 | 4.5E−02 | 1 |
| X-18945 | one | 195 | 0.35 | 4.5E−02 | 1 |
| 2-hydroxy- | one | 143 | 0.40 | 4.6E−02 | 1 |
| Acetaminophen | | | | | |
| sulfate* | | | | | |
| phosphate | one | 206 | 0.34 | 4.7E−02 | 1 |
| X-11491 | one | 127 | 0.48 | 4.7E−02 | 1 |
| Dimethyl- | one | 206 | −0.34 | 4.8E−02 | 1 |
| arginine | | | | | |
| (SDMA + | | | | | |
| ADMA) | | | | | |
| catechol | one | 206 | −0.34 | 5.0E−02 | 1 |
| sulfate | | | | | |

Table 5 is shown below. For Table 5, the numerator refers to a Metabolon compound identifier in the numerator; the denominator refers to a Metabolon compound identifier in the denominator (where ‘one’ indicates ‘no ratio’); the term ncases refers to the number of data points used in the statistics; the term OR refers to the odds ratio of the association with ACR, per 1 standard deviation change in predictor; the p-value refers to the p-value of the association with ACR; and the p-gain refers to the p-gain of the association with ACR (when using ratios)

TABLE 5
Logistic regression [ACR = f(Mᵢ)] using all samples
with matching biopsy and available RNA signature

| numerator | denominator | ncases | OR | p-value | p-gain |
|---|---|---|---|---|---|
| quinolinate | 4-hydroxy- | 206 | 2.67 | 7.3E−07 | 1,139 |
| | mandelate | | | | |
| quinolinate | xylitol | 205 | 2.56 | 9.1E−07 | 909 |
| 3-sialyl- | xanthosine | 201 | 2.74 | 1.7E−06 | 5,814 |
| lactose | | | | | |
| neopterin | xanthosine | 195 | 2.65 | 2.7E−06 | 2,631 |
| X-17366 | xanthosine | 197 | 2.59 | 4.5E−06 | 2,194 |
| neopterin | N1-methyl- | 198 | 2.47 | 5.4E−06 | 1,315 |
| | guanosine | | | | |
| X-18486 | xanthosine | 201 | 2.61 | 6.1E−06 | 1,626 |
| kynurenate | xanthosine | 201 | 2.62 | 7.1E−06 | 1,392 |
| X-12690 | xanthosine | 201 | 2.51 | 1.2E−05 | 818 |
| 3-sialyl- | X-16397 | 206 | 2.31 | 1.9E−05 | 1,499 |
| lactose | | | | | |
| pregnen- | droxypregnenolone | 163 | 2.39 | 1.2E−04 | 992 |
| diol | disulfate | | | | |
| disulfate* | | | | | |
| proline | one | 204 | 1.89 | 1.5E−04 | 1 |
| leucine | one | 203 | 1.97 | 1.8E−04 | 1 |
| 1,5-anhydro- | one | 165 | 2.30 | 2.1E−04 | 1 |
| glucitol | | | | | |
| (1,5-AG) | | | | | |
| isoleucine | one | 202 | 1.87 | 5.6E−04 | 1 |
| 4-hydroxy- | one | 206 | 0.53 | 7.7E−04 | 1 |
| phenylacetate | | | | | |
| quinolinate | one | 205 | 1.79 | 8.3E−04 | 1 |
| N-methyl- | one | 145 | 2.07 | 8.8E−04 | 1 |
| acetaminophen | | | | | |
| sulfate 1* | | | | | |
| X-16940 | one | 201 | 0.55 | 9.3E−04 | 1 |
| pipecolate | one | 199 | 1.77 | 1.0E−03 | 1 |
| xylitol | one | 206 | 0.55 | 1.2E−03 | 1 |
| X-12117 | one | 201 | 1.72 | 1.4E−03 | 1 |
| gentisate | one | 201 | 0.55 | 1.6E−03 | 1 |
| xanthine | one | 206 | 0.55 | 1.7E−03 | 1 |
| X-13723 | one | 198 | 0.52 | 1.9E−03 | 1 |
| uracil | one | 206 | 0.57 | 1.9E−03 | 1 |
| 4-hydroxy- | one | 205 | 0.58 | 1.9E−03 | 1 |
| mandelate | | | | | |
| homovanillate | one | 205 | 0.58 | 2.0E−03 | 1 |
| (HVA) | | | | | |
| 3-(N-acetyl- | one | 119 | 2.25 | 2.3E−03 | 1 |
| L-cystein-S-yl) | | | | | |
| acetaminophen* | | | | | |
| phenylalanine | one | 204 | 1.67 | 2.8E−03 | 1 |
| stachydrine | one | 206 | 1.77 | 2.8E−03 | 1 |
| valine | one | 203 | 1.67 | 3.3E−03 | 1 |
| trigonelline | one | 206 | 0.59 | 3.5E−03 | 1 |
| (N′-methyl- | | | | | |
| nicotinate) | | | | | |
| lysine | one | 204 | 1.67 | 3.8E−03 | 1 |
| X-12855 | one | 174 | 1.76 | 4.1E−03 | 1 |
| X-17320 | one | 128 | 0.49 | 4.5E−03 | 1 |
| X-12230 | one | 177 | 0.54 | 4.5E−03 | 1 |
| paraxanthine | one | 131 | 0.51 | 5.1E−03 | 1 |
| methionine | one | 204 | 1.61 | 5.8E−03 | 1 |
| kynurenine | one | 204 | 1.67 | 5.8E−03 | 1 |
| 3,4-dihydroxy- | one | 203 | 0.62 | 6.1E−03 | 1 |
| phenylacetate | | | | | |
| X-16570 | one | 203 | 0.60 | 6.2E−03 | 1 |
| 2-methoxy- | one | 101 | 2.09 | 7.1E−03 | 1 |
| acetaminophen | | | | | |
| sulfate* | | | | | |
| neopterin | one | 200 | 1.57 | 7.1E−03 | 1 |
| X-12221 | one | 195 | 0.61 | 7.3E−03 | 1 |
| X-19434 | one | 110 | 2.07 | 8.2E−03 | 1 |
| 5-hydroxy- | one | 199 | 0.61 | 9.6E−03 | 1 |
| methyl- | | | | | |
| 2-furoic acid | | | | | |
| N2-methyl- | one | 202 | 0.62 | 9.8E−03 | 1 |
| guanosine | | | | | |
| xanthosine | one | 201 | 0.61 | 9.9E−03 | 1 |
| p-acetamido- | one | 137 | 1.83 | 1.0E−02 | 1 |
| phenyl- | | | | | |
| glucuronide | | | | | |
| Acetyl- | one | 206 | 1.57 | 1.0E−02 | 1 |
| carnitine | | | | | |
| X-17366 | one | 200 | 1.58 | 1.1E−02 | 1 |
| kynurenate | one | 204 | 1.55 | 1.1E−02 | 1 |
| ornithine | one | 204 | 1.54 | 1.1E−02 | 1 |
| X-14625 | one | 200 | 1.57 | 1.1E−02 | 1 |
| Propionyl- | one | 193 | 1.59 | 1.2E−02 | 1 |
| carnitine | | | | | |
| 2-methyl- | one | 198 | 1.62 | 1.2E−02 | 1 |
| Butyryl- | | | | | |
| carnitine (C5) | | | | | |
| Isobutyryl- | one | 200 | 0.63 | 1.2E−02 | 1 |
| glycine | | | | | |
| myo-inositol | one | 206 | 1.58 | 1.3E−02 | 1 |
| Imidazole | one | 203 | 0.64 | 1.4E−02 | 1 |
| lactate | | | | | |
| urate | one | 206 | 1.58 | 1.4E−02 | 1 |
| Isovaleryl- | one | 203 | 0.64 | 1.4E−02 | 1 |
| glycine | | | | | |
| glucose | one | 206 | 1.50 | 1.5E−02 | 1 |
| mannose | one | 206 | 1.50 | 1.6E−02 | 1 |
| X-12039 | one | 205 | 0.65 | 1.7E−02 | 1 |
| X-12107 | one | 187 | 0.61 | 1.8E−02 | 1 |
| 3-hydroxy- | one | 133 | 1.60 | 1.8E−02 | 1 |
| kynurenine | | | | | |
| ribitol | one | 205 | 0.67 | 1.9E−02 | 1 |
| X-11843 | one | 130 | 1.70 | 1.9E−02 | 1 |
| 4-hydroxy- | one | 206 | 0.63 | 2.0E−02 | 1 |
| benzoate | | | | | |
| arginine | one | 191 | 1.51 | 2.0E−02 | 1 |
| 5 alpha-androstan- | one | 83 | 1.94 | 2.1E−02 | 1 |
| 3-beta,17-beta- | | | | | |
| diol disulfate | | | | | |
| isocitrate | one | 206 | 0.68 | 2.1E−02 | 1 |
| X-12685 | one | 187 | 0.62 | 2.2E−02 | 1 |
| 3-(cystein- | one | 106 | 1.80 | 2.2E−02 | 1 |
| S-yl)acetamino- | | | | | |
| phen* | | | | | |
| 3- | one | 206 | 0.66 | 2.2E−02 | 1 |
| aminoisobutyrate | | | | | |
| X-17735 | one | 194 | 0.64 | 2.2E−02 | 1 |
| X-12193 | one | 204 | 0.66 | 2.3E−02 | 1 |
| X-16674 | one | 192 | 0.65 | 2.3E−02 | 1 |
| allantoin | one | 197 | 1.52 | 2.4E−02 | 1 |
| X-17185 | one | 161 | 0.54 | 2.4E−02 | 1 |
| X-12114 | one | 204 | 1.51 | 2.5E−02 | 1 |
| X-17369 | one | 74 | 2.10 | 2.5E−02 | 1 |
| hydantoin- | one | 190 | 1.47 | 2.6E−02 | 1 |
| 5-propionic | | | | | |
| acid | | | | | |
| trans- | one | 204 | 0.68 | 2.7E−02 | 1 |
| urocanate | | | | | |
| xylulose | one | 205 | 0.68 | 2.7E−02 | 1 |
| 1,7-dimethyl- | one | 187 | 0.67 | 2.7E−02 | 1 |
| urate | | | | | |
| X-12849 | one | 206 | 0.69 | 2.8E−02 | 1 |
| lactate | one | 202 | 1.44 | 2.8E−02 | 1 |
| tyrosine | one | 204 | 1.47 | 2.8E−02 | 1 |
| X-15461 | one | 200 | 0.67 | 2.8E−02 | 1 |
| 3-(3-hydroxy- | one | 141 | 0.62 | 2.8E−02 | 1 |
| phenyl)propionate | | | | | |
| 3-sialyllactose | one | 206 | 1.44 | 2.8E−02 | 1 |
| citrate | one | 206 | 0.68 | 2.8E−02 | 1 |
| pantothenate | one | 203 | 0.68 | 3.1E−02 | 1 |
| cystine | one | 164 | 1.54 | 3.1E−02 | 1 |
| X-18965 | one | 201 | 0.68 | 3.1E−02 | 1 |
| X-12726 | one | 204 | 0.68 | 3.4E−02 | 1 |
| hypoxanthine | one | 206 | 0.68 | 3.4E−02 | 1 |
| X-17335 | one | 205 | 0.69 | 3.5E−02 | 1 |
| X-18486 | one | 206 | 1.48 | 3.6E−02 | 1 |
| 3,5-dihydroxy- | one | 194 | 0.65 | 3.6E−02 | 1 |
| benzoic acid | | | | | |
| X-15484 | one | 142 | 1.58 | 3.7E−02 | 1 |
| X-18887 | one | 205 | 1.45 | 3.7E−02 | 1 |
| N-(2-furoyl)- | one | 206 | 0.69 | 3.8E−02 | 1 |
| glycine | | | | | |
| X-11668 | one | 199 | 0.70 | 3.8E−02 | 1 |
| X-13850 | one | 139 | 1.53 | 3.9E−02 | 1 |
| guanine | one | 198 | 0.69 | 3.9E−02 | 1 |
| glutamine | one | 204 | 1.44 | 3.9E−02 | 1 |
| mesaconate | one | 202 | 0.69 | 3.9E−02 | 1 |
| (methyl- | | | | | |
| fumarate) | | | | | |
| X-17361 | one | 192 | 0.68 | 4.0E−02 | 1 |
| taurine | one | 193 | 0.69 | 4.4E−02 | 1 |
| X-17398 | one | 153 | 0.62 | 4.4E−02 | 1 |
| 4-acetamino- | one | 169 | 1.45 | 4.7E−02 | 1 |
| phen sulfate | | | | | |
| X-18945 | one | 195 | 1.42 | 4.8E−02 | 1 |
| X-12738 | one | 128 | 0.62 | 4.9E−02 | 1 |
| 2-hydroxy- | one | 143 | 1.52 | 4.9E−02 | 1 |
| acetaminophen | | | | | |
| sulfate* | | | | | |
| phosphate | one | 206 | 1.43 | 4.9E−02 | 1 |
| X-12747 | one | 132 | 0.67 | 5.0E−02 | 1 |

Table 6 is shown below. For Table 6, the numerator refers to a Metabolon compound identifier in the numerator; the denominator refers to a Metabolon compound identifier in the denominator (where ‘one’ indicates ‘no ratio’); the term ncases refers to the number of data points used in the statistics; the term OR(metabolite) refers to the odds ratio of the association with ACR, per 1 standard deviation change in predictor; the p-value(metabolite) refers to the p-value for a metabolite; the p-gain(metabolite refers to the p-gain of the metabolite (when using ratios); the term OR(RNA signature) refers to the odds ratio of the RNA signature; and the p-value(RNA signature) refers to the p-value of the RNA signature.

TABLE 6
Logistic regression [ACR = f(Mi, RNA_signature)] using all
samples with matching biopsy and available RNA signature

| | | | Metabolite | | | RNA signature | |
|---|---|---|---|---|---|---|---|
| numerator | denominator | ncases | OR | p-value | p-gain | OR | p-value |
| 3-sialyl- | xanthosine | 200 | 3.81 | 1.6E−06 | 3,702 | 3.25 | 3.8E−08 |
| lactose | | | | | | | |
| X-12690 | xanthosine | 200 | 3.72 | 4.3E−06 | 1,024 | 3.17 | 1.0E−08 |
| X-12715 | 4-hydroxy- | 205 | 3.02 | 1.0E−05 | 1,199 | 3.42 | 4.7E−09 |
| | phenyl- | | | | | | |
| | acetate | | | | | | |
| X-12225 | homo- | 202 | 3.13 | 1.1E−05 | 1,255 | 3.38 | 2.5E−09 |
| | vanillate | | | | | | |
| | (HVA) | | | | | | |
| X-11334 | xanthosine | 200 | 2.81 | 3.8E−05 | 1,189 | 3.17 | 3.0E−09 |
| X-11945 | xanthosine | 200 | 2.95 | 3.9E−05 | 784 | 3.02 | 1.0E−08 |
| X-12117 | one | 200 | 2.66 | 6.9E−05 | 1 | 3.25 | 1.7E−09 |
| leucine | one | 202 | 2.14 | 5.4E−04 | 1 | 2.81 | 7.2E−09 |
| quinolinate | one | 204 | 2.16 | 7.9E−04 | 1 | 2.91 | 1.8E−09 |
| stachydrine | one | 205 | 2.21 | 9.4E−04 | 1 | 3.00 | 1.7E−09 |
| X-16940 | one | 200 | 0.50 | 1.3E−03 | 1 | 2.83 | 1.5E−08 |
| Phenyl- | one | 203 | 2.03 | 1.4E−03 | 1 | 2.86 | 1.8E−09 |
| alanine | | | | | | | |
| X-17366 | one | 199 | 2.07 | 1.8E−03 | 1 | 3.00 | 1.3E−09 |
| proline | one | 203 | 1.95 | 1.9E−03 | 1 | 2.69 | 4.9E−09 |
| methionine | one | 203 | 2.04 | 1.9E−03 | 1 | 2.90 | 1.4E−09 |
| lysine | one | 203 | 1.92 | 2.1E−03 | 1 | 2.84 | 1.8E−09 |
| trigonelline | one | 205 | 0.50 | 2.7E−03 | 1 | 2.85 | 1.8E−09 |
| (N′-methyl- | | | | | | | |
| nicotinate) | | | | | | | |
| 1,5-anhydro- | one | 165 | 2.44 | 2.8E−03 | 1 | 3.43 | 6.5E−08 |
| glucitol | | | | | | | |
| (1,5-AG) | | | | | | | |
| X-18887 | one | 204 | 1.93 | 3.3E−03 | 1 | 2.99 | 9.8E−10 |
| neopterin | one | 199 | 1.81 | 3.4E−03 | 1 | 2.79 | 2.3E−09 |
| lactate | one | 201 | 1.88 | 3.9E−03 | 1 | 2.89 | 2.3E−09 |
| X-12690 | one | 204 | 1.90 | 4.4E−03 | 1 | 3.00 | 6.6E−10 |
| isoleucine | one | 201 | 1.95 | 4.5E−03 | 1 | 2.74 | 6.3E−09 |
| X-18486 | one | 205 | 2.06 | 5.1E−03 | 1 | 2.93 | 7.3E−10 |
| kynurenine | one | 203 | 1.87 | 5.4E−03 | 1 | 2.86 | 3.4E−09 |
| 3-sialyl- | one | 205 | 1.78 | 5.8E−03 | 1 | 2.93 | 8.6E−10 |
| lactose | | | | | | | |
| gentisate | one | 200 | 0.52 | 5.9E−03 | 1 | 2.67 | 1.1E−08 |
| myo- | one | 205 | 1.94 | 6.0E−03 | 1 | 2.94 | 1.6E−09 |
| inositol | | | | | | | |
| 5-oxopro- | one | 204 | 1.86 | 6.8E−03 | 1 | 3.03 | 6.4E−10 |
| line | | | | | | | |
| xanthine | one | 205 | 0.54 | 7.1E−03 | 1 | 2.79 | 4.4E−09 |
| kynurenate | one | 203 | 1.80 | 7.4E−03 | 1 | 2.79 | 2.4E−09 |
| ornithine | one | 203 | 1.79 | 7.6E−03 | 1 | 2.84 | 2.1E−09 |
| X-12364 | one | 188 | 1.92 | 8.4E−03 | 1 | 2.99 | 1.7E−08 |
| X-12125 | one | 204 | 1.81 | 9.4E−03 | 1 | 2.98 | 1.1E−09 |
| X-12100 | one | 202 | 1.87 | 9.5E−03 | 1 | 3.15 | 6.5E−10 |
| X-12637 | one | 203 | 1.76 | 1.0E−02 | 1 | 2.98 | 1.5E−09 |
| X-12855 | one | 173 | 1.92 | 1.0E−02 | 1 | 2.58 | 1.7E−07 |
| valine | one | 202 | 1.75 | 1.1E−02 | 1 | 2.72 | 4.2E−09 |
| X-13737 | one | 203 | 1.76 | 1.2E−02 | 1 | 2.96 | 9.7E−10 |
| arginine | one | 190 | 1.78 | 1.2E−02 | 1 | 2.75 | 5.5E−09 |
| 4-hydroxy- | one | 205 | 0.57 | 1.2E−02 | 1 | 2.69 | 5.0E−09 |
| phenyl- | | | | | | | |
| acetate | | | | | | | |
| pipecolate | one | 198 | 1.72 | 1.3E−02 | 1 | 2.69 | 4.1E−09 |
| 1-(3-amino- | one | 195 | 1.81 | 1.3E−02 | 1 | 2.90 | 2.0E−09 |
| propyl)-2- | | | | | | | |
| pyrrolidone | | | | | | | |
| X-12221 | one | 194 | 0.57 | 1.3E−02 | 1 | 2.67 | 9.0E−09 |
| homo- | one | 204 | 0.57 | 1.3E−02 | 1 | 2.67 | 5.1E−09 |
| vanillate | | | | | | | |
| (HVA) | | | | | | | |
| X-13723 | one | 197 | 0.54 | 1.4E−02 | 1 | 2.63 | 7.6E−09 |
| X-12707 | one | 205 | 1.76 | 1.4E−02 | 1 | 3.10 | 5.2E−10 |
| X-12039 | one | 204 | 0.59 | 1.4E−02 | 1 | 2.80 | 1.6E−09 |
| phosphate | one | 205 | 1.75 | 1.5E−02 | 1 | 2.90 | 1.1E−09 |
| X-16947 | one | 202 | 1.67 | 1.6E−02 | 1 | 2.84 | 8.0E−10 |
| cysteine | one | 197 | 1.65 | 1.6E−02 | 1 | 3.02 | 1.1E−09 |
| X-18475 | one | 194 | 1.76 | 1.7E−02 | 1 | 2.87 | 3.3E−09 |
| X-14625 | one | 199 | 1.64 | 1.8E−02 | 1 | 2.74 | 1.6E−09 |
| urate | one | 205 | 1.73 | 1.9E−02 | 1 | 2.77 | 1.5E−09 |
| X-12906 | one | 203 | 1.70 | 2.2E−02 | 1 | 3.04 | 1.6E−09 |
| uracil | one | 205 | 0.61 | 2.2E−02 | 1 | 2.62 | 3.2E−09 |
| X-12685 | one | 186 | 0.56 | 2.2E−02 | 1 | 2.64 | 1.1E−08 |
| Vanillyl- | one | 203 | 1.65 | 2.4E−02 | 1 | 3.12 | 1.1E−09 |
| mandelate | | | | | | | |
| (VMA) | | | | | | | |
| X-12225 | one | 203 | 1.60 | 2.6E−02 | 1 | 2.98 | 9.7E−10 |
| Acetyl- | one | 205 | 1.61 | 2.6E−02 | 1 | 2.77 | 3.1E−09 |
| carnitine | | | | | | | |
| Homo- | one | 201 | 1.55 | 2.7E−02 | 1 | 2.85 | 1.8E−09 |
| citrulline | | | | | | | |
| glutamine | one | 203 | 1.67 | 2.7E−02 | 1 | 2.77 | 1.5E−09 |

The level of significance for association of a metabolite after Bonferroni correction at a nominal level of significance of 0.05 is a P<6.7×10⁻⁵ (=0.05/749). Some studies suggest that testing all possible combinations of ratios between metabolite concentrations may reveal new and biologically relevant associations in an unbiased approach (Dehaven et al., J Cheminform 2: 9 (2010). Therefore, all pairs of metabolite ratios were included in the association tests. For the ratios, the significance level after Bonferroni correction was P<1.8×10⁻⁷ (=0.05/(749*748/2)). Note that since the data are log-scaled, the symmetry log(a/b)=−log(b/a) halves the multiple testing burden. A ratio between two metabolites where one already shows a strong association signal may suggest false positive implication of the other in the association. Therefore, the inventors also required that the P-gain statistic to be significant after Bonferroni correction. The P-gain is defined as the change in the P values of the association of the two single metabolites when compared to their ratio. An association of a ratio is thus considered significant if P-gain>7,490 (=10*749) and P-value<1.8×10⁻⁷. Due to the inherent correlations among metabolic traits, the less conservative false discovery rate (FDR), introduced by Benjamini-Hochberg (Benjamini et al., J R Stat Soc Series B Methodol 57: 289-300 (1995)) is generally used in metabolomics studies. In order not to miss metabolites of potential biological interest we report associations that are significant at the FDR level of 5% in Table 1, and for all nominal associations in Tables 2, 3, 4, 5, and 6.

In summary, the urine metabolomics study discovered that the ratio of 3-sialyllactose to xanthosine and the ratio of quinolinate to X-16397 were best associated with ACR biopsy diagnosis.

### Example 2: Urine Samples for Metabolomics

From a total of 4300 urine samples prospectively collected from the 485 kidney graft recipients (patients) enrolled in the parent CTOT-04 study, 1518 urine samples were selected from 242 patients for metabolomics (FIG. 1) to include: (i) all 298 urine samples matched to 298 kidney allograft biopsies (urine samples collected from 3 days before to 1 day after the biopsy); (ii) all 808 sequential urine samples preceding a biopsy diagnosis; and (iii) all 412 urine samples from clinically stable patients who provided >10 sequential samples in the first 400 days of transplantation. High-quality data was obtained for 1516 urine samples from 241 kidney allograft recipients after exclusion of one patient and two samples (Example 1) regarding 749 different metabolites from 65 metabolic pathways, including 368 metabolites of unknown identity.

### Example 3: Urine Metabolites and ACR

Metabolite data from 50 urine samples matched to 50 ACR biopsies from 36 patients and 198 urine samples matched to 198 No rejection biopsies from 149 patients were analyzed to determine whether metabolite profiles distinguish ACR biopsies from No Rejection biopsies.

Table 1 lists transplant recipient's characteristics such as age, gender, ethnicity, race and BMI. Among the 50 ACR biopsies graded using the Banff schema, 23 were graded as ACR grade IA, 11 were graded as ACR grade IB, 12 were graded as ACR grade IIA, 3 were graded as ACR grade IIB and 1 was graded as ACR grade III.

The 198 biopsies classified as No Rejection biopsies did not show histologic features of ACR, AMR, Borderline, bacterial infection/pyelonephritis, CMV, BKV/polyoma nephropathy or post-transplant lymphoproliferative disease. However, several of the No Rejection biopsies displayed histological changes consistent with ATN (n=79), tubular atrophy (n=75), interstitial fibrosis (n=67), glomerulosclerosis (n=30), vascular narrowing (n=20), calcineurin inhibitor toxicity (n=18), and/or recurrent disease (n=2). Also, several of the biopsies showed more than one histological abnormality such as the presence of both interstitial fibrosis and tubular atrophy.

Kidney allograft function, measured at the time of biopsy, showed that the graft function was significantly inferior in the ACR biopsy group compared to the No Rejection biopsy group. As summarized in Table 1, the mean (+SD) serum creatinine level at the time of kidney allograft biopsy in the ACR biopsy group was 3.7+2.9 mg/dl (number of measurements=37) and was 2.5+2.3 mg/dl (number of measurements=146) in the No Rejection biopsy group (Mann Whitney P=0.0003). The mean (+SD) estimated glomerular filtration rate (eGFR) in the ACR biopsy group was 30+17 mL/min (number of measurements=37) compared to 43+23 mL/min (number of measurements=146) in the No Rejection biopsy group (Mann Whitney P=0.003).

Metabolite data was also analyzed from urine samples matched to antibody-mediated rejection, borderline changes, BKV nephropathy, or other biopsy findings. Due to the small group sizes and the resulting lack of statistical power, results from these analyses are not included herein.

Table 7 lists all metabolites and ratios of metabolites in urine that distinguished ACR biopsies from No Rejection biopsies at a false discovery rate of 5% (Benjamini et al., J R Stat Soc Series B Methodol 57: 289-300 (1995).

TABLE 7
Metabolite ratios and metabolite concentrations distinguishing
acute cellular rejection biopsies from no rejection biopsies
at a false discovery rate of 5%.

| Metabolite Ratio or Metabolite | N^{a} | beta^{b} | P-value^{c} | P-gain^{c} |
|---|---|---|---|---|
| 3-Sialyllactose/Xanthosine | 242 | 0.86 | 5.0 × 10⁻⁸ | 201,100 |
| Neopterin/Xanthosine | 234 | 0.90 | 2.0 × 10⁻⁸ | 72,220 |
| 3-(3-aminocarboxypropyl) | 242 | 0.83 | 1.6 × 10⁻⁷ | 61,644 |
| Uridine/Xanthosine | | | | |
| 3-Sialyllactose/X-16397^{d} | 247 | 0.77 | 5.9 × 10⁻⁷ | 26,062 |
| Quinolinate/X-16397^{d} | 248 | 0.89 | 7.3 × 10⁻⁹ | 25,031 |
| Quinolinate/4-hydroxy- | 248 | 0.88 | 1.1 × 10⁻⁸ | 16,954 |
| mandelate | | | | |
| Neopterin/X-16570^{d} | 235 | 0.84 | 9.7 × 10⁻⁸ | 14,493 |
| Neopterin/N1-Methyl- | 238 | 0.83 | 1.2 × 10⁻⁷ | 11,361 |
| guanosine | | | | |
| Proline | 245 | 0.63 | 4.9 × 10⁻⁵ | — |
| Quinolinate | 247 | 0.59 | 1.8 × 10⁻⁴ | — |
| Isoleucine | 244 | 0.58 | 2.1 × 10⁻⁴ | — |
| X-13723^{d} | 235 | −0.57 | 3.7 × 10⁻⁴ | — |
| X-12117^{d} | 242 | 0.55 | 4.9 × 10⁻⁴ | — |
| 1,2,3 Benzenetriol | 241 | −0.55 | 5.8 × 10⁻⁴ | — |
| sulfate | | | | |
| Leucine | 246 | 0.52 | 9.9 × 10⁻⁴ | — |
| Pipecolate | 239 | 0.52 | 1.1 × 10⁻³ | — |
| Paraxanthine | 158 | −0.63 | 1.1 × 10⁻³ | — |
| 1,5-Anhydroglucitol | 197 | 0.57 | 1.1 × 10⁻³ | — |
| X-19434^{d} | 126 | 0.77 | 1.3 × 10⁻³ | — |
| Kynurenate | 245 | 0.51 | 1.3 × 10⁻³ | — |
| Neopterin | 240 | 0.51 | 1.4 × 10⁻³ | — |
| Myo-inositol | 248 | 0.49 | 1.7 × 10⁻³ | — |
| Gentisate | 243 | −0.50 | 1.8 × 10⁻³ | — |
| Valine | 243 | 0.49 | 1.9 × 10⁻³ | — |
| N-Methyl-acetaminophen | | | | |
| sulfate 1 | 175 | 0.58 | 2.5 × 10⁻³ | — |
| Xylitol | 248 | −0.47 | 2.6 × 10⁻³ | — |

^{a}N is the number of urine samples with valid metabolite data following analysis of 248 urine samples from 185 patients matched to either ACR biopsies (number of urine samples = 50; number of patients = 36) or No rejection biopsies (number of urine samples = 198; number of patients = 149). One No Rejection biopsy associated sample contained insufficient material for metabolomics, and reduced the number of urine samples from 199 to 198, and the number of patients from 150 to 149, reported in the parent CTOT-04 study (Suthanthiran et al., N Engl J Med 369: 20-31 (2013)). Urine samples matched to biopsies classified as Borderline changes (number of urine samples = 27; number of patients = 25), AMR (number of urine samples = 13: number of patients = 11) or Other biopsies (number of urine samples = 9; number of patients = 8) were also excluded from data analysis since the objective was to determine whether urine metabolite profiles distinguish ACR biopsies from No Rejection biopsies. The number of patients with a biopsy diagnosis exceeds the 190 patients providing 298 biopsy matched urine samples since several patients had multiple biopsies with different biopsy diagnoses.
^{b}beta is the slope of the linear model fitted to log₁₀-transformed and z-scored metabolite data (ion-counts, coding of the independent variable: 0 = No Rejection, 1 = ACR).
^{c}P-value and P-gain refer to the linear model “metabolite or metabolite ratio = f(ACR)”; associations listed here are limited to those with a false discovery rate (FDR) of <5% for single metabolite associations; P value and P-gain that are significant at the p < 0.05 level after Bonferroni correction are shown in bold. See Table 2 for all nominally significant associations (i.e., with no adjustment for the number of tests) by linear regression and Table 3 for all nominally significant associations by logistic regression. Although statistically less powered, the top associations that emerged from these alternative statistical analyses were essentially the same, demonstrating the robustness of the association of metabolite profiles with ACR biopsy diagnosis.
^{d}Any metabolite of unknown structural identity is labeled as X-nnnnn (e.g., X-16397).

Tables 2 and 3 provide all nominally significant associations (i.e., with no adjustment for the number of tests) by linear regression (Table 2) or logistic regression (Table 3).

The ratio of the concentrations of the metabolites 3-sialyllactose to xanthoine (3SL/X) in the urine supernatant was strongly associated with ACR (P=5.0×10⁻⁸) and this ratio had the highest increase in the strength of association (P-gain=2.0×10⁵, compared to the association of the single metabolites in the ratio). For a definition of the P-gain statistic see Example 1 and Petersen et al. (BMC Bioinformatics 13: 120 (2012)). The 3SL/X ratio was statistically significant even under conservative Bonferroni correction of the P value and the P-gain.

The diagnostic signature of the ratio of 3SL/X was not associated with age (P=0.95, Spearman regression), gender (P=0.52, Mann-Whitney test), or ethnicity (P=0.19, Kruskal-Wallis-Analysis of Variance). The signature was only weakly associated with eGFR (P=0.054, Spearman regression, Sᵣ=−0.168) and the 3SL/X ratio continued to be diagnostic of ACR after adjusting for eGFR.

### Example 4: A Composite Metabolite Signature of ACR

The ratio of 3SL/X showed by far the strongest P-gain and was therefore considered the prime candidate for a metabolite signature. After controlling for the 3SL/X ratio in a logistic regression model predicting ACR, the next strongest independent predictor of ACR was the ratio of quinolinate to X-16397, a metabolite of unknown identity. Analysis of the receiver-operating-characteristics (ROC) curve showed that the area under the curve (AUC) for the 3SL/X was 0.75, and the signature at the Youden-index (Le, Stat Methods Med Res 15: 571-584 (2006)) based cutoff was diagnostic of ACR with a specificity of 76% and a sensitivity of 59% (Table 8).

TABLE 8
Performance characteristics of the metabolite signature,
RNA diagnostic signature and the combined signature discriminating
acute cellular rejection biopsies from no rejection biopsies.

| | | Specificity | Sensitivity | Youden |
|---|---|---|---|---|
| Signature^{a, b} | AUC [95%] | (1-FP, %) | (TP, %) | index, % |
| Log(3-sialyllactose/xanthosine) | 0.75 [0.60] | 76 | 59 | 36 |
| Log(quinolinate/X-16397) | 0.71 [0.60] | 88 | 51 | 39 |
| Log(3-sialyllactose/xanthosine) + | 0.81 [0.77] | 71 | 82 | 53 |
| 0.9513 * log(quinolinate/X- | | | | |
| 16397) | | | | |
| RNA signature | 0.84 [0.60] | 72 | 85 | 56 |
| RNA signature + | 0.91 [0.85] | 82 | 87 | 69 |
| 1.1164 * log(3-sialyllactose/ | | | | |
| xanthosine) | | | | |
| RNA signature + | 0.88 [0.85] | 77 | 85 | 62 |
| 0.8932 * log(quinolinate/X- | | | | |
| 16397) | | | | |
| RNA-signature + | 0.93 [0.92] | 84 | 90 | 74 |
| 1.1164 * log(3-sialyllactose/ | | | | |
| xanthosine) + | | | | |
| 0.6937 * log(quinolinate/X- | | | | |
| 16397) | | | | |

^{a}The signature that optimizes the area under the curve (AUC) is given together [in square brackets] with the largest AUC that can be observed by chance (P = 0.05) when the last term of the signature is randomized in the optimization procedure (95^{th} percentile of 1000 randomizations). Sensitivity and specificity based on Youden index maximizing the difference between the true positive (TP) and the false positive (FP) rate for the signatures are listed.
^{b}To make performance characteristics of the signatures comparable, the values shown in Table 8 were all computed using 198 urine samples from 154 patients (39 ACR biopsy urine samples from 31 patients and 159 No Rejection biopsy matched urine samples from 123 patients) that contained both valid metabolite data and urinary cell RNA signature score. This resulted in the RNA signature having slightly different values in the metabolomics study compared to the values reported in the parent CTOT-04 study in which 43 ACR biopsy matched urine samples from 34 patients and 163 No rejection biopsy matched urine samples from 126 patients were analyzed and the RNA signature was diagnostic of ACR with a specificity of 78% and a sensitivity of 79% and the AUC by ROC curve analysis was 0.85. Suthanthiran et al., N Engl J Med 369: 20-31 (2013).

The ratio of quinolinate to X-16397 also showed a strong association in the logistic model and a linear combination of the ratios of 3SL/X and quinolinate to X-16397 increased the AUC from 0.75 to 0.81 and the Youden index from 36% to 53% (Table 8).

The inventors examined whether any of the other metabolites and metabolite ratios reported in Table 7 outperformed the combined metabolite signature of the ratios of 3SL/X and quinolinate to X-16397, and none of the other metabolites or metabolite ratios did.

### Example 5: A Composite Metabolite and the RNA Signature of ACR

The performance characteristics of the newly discovered metabolite signatures were investigated in combination with the inventor's previously established diagnostic signature of 18S rRNA and of 18S-normalized CD3ε RNA and IP-10 RNA measures in urinary cells. For consistency in the comparisons between the metabolite signatures and the RNA signature urine samples were only used for which both metabolite and RNA data were available for all computed signatures. Tables 4 and 5 provide metabolite association data when samples without RNA data were excluded.

To identify the combined metabolite- and RNA-based signature, a logistic regression was performed with ACR=f(Mᵢ, RNA-signature), where Mᵢ represents a log-scaled metabolite concentration or a log-scaled ratio between two metabolite concentrations. The log-scaled ratio of 3SL/X had the highest log-odds ratio (1.34, P=1.6×10⁻⁶) after inclusion of the RNA signature in the model and thus represented the best candidate for a combined metabolite-RNA signature (Table 6). The linear combination between log (3SL/X) and the RNA-signature that maximized the AUC were selected. The resulting combined metabolite-RNA signature was:

RNA-signature+1.1164*log(3SL/X).

Compared to the RNA-signature alone (AUC=0.84), the combined metabolite-RNA signature had a significantly higher AUC of 0.91 (significance determined by random sampling, an AUC of 0.85 or below was observed in 95% of 1,000 random samplings) (Table 8).

With the RNA signature and the 3SL/X ratio included in the logistic analysis, the quinolinate/X-16397 ratio was the next strongest predictor. With this additional metabolite ratio, the resulting combined signature that maximized the AUC was the following:

RNA-signature+1.1164*log(3SL/X)+0.6937*log(quinolinate/X-16397).

This combined two-metabolite-ratios-RNA signature increased the AUC of the one-metabolite-ratio-RNA signature to 0.93 (Table 8). This composite signature was diagnostic of ACR with a specificity of 84% and a sensitivity of 90%. Taken together, these results show that adding metabolite information to the RNA signature substantially improves its diagnostic utility as indicated by the more than 30% increase in the Youden index from 56% for the RNA signature alone to 74% for the combined two-metabolite-ratio-RNA signature.

Among the 39 ACR biopsies from 31 patients (Table 8), 34 biopsies were for-cause biopsies and 5 biopsies were surveillance biopsies. Among the 159 No Rejection biopsies from 123 patients, 104 biopsies were for-cause biopsies, and 55 biopsies were surveillance biopsies. The composite metabolite and RNA signature distinguished the 34 for-cause ACR biopsies from the 159 for-cause No Rejection biopsies (P=1.6×10⁻¹⁷); the composite metabolite and RNA signature also distinguished the five surveillance ACR biopsies from the 55 surveillance No Rejection biopsies (P=0.0002).

### Example 6: Prognostic Performance of the Signatures

The signatures were examined to ascertain whether, in addition to being diagnostic of ACR, the signatures could predict future occurrence of an ACR. For this analysis, the day of kidney biopsy was designated as day 0 and a total of 337 urine samples with both urine metabolite data and urinary cell RNA data and collected up to one year prior to an ACR biopsy or a No Rejection biopsy were analyzed to investigate whether the signatures predict future ACR biopsies.

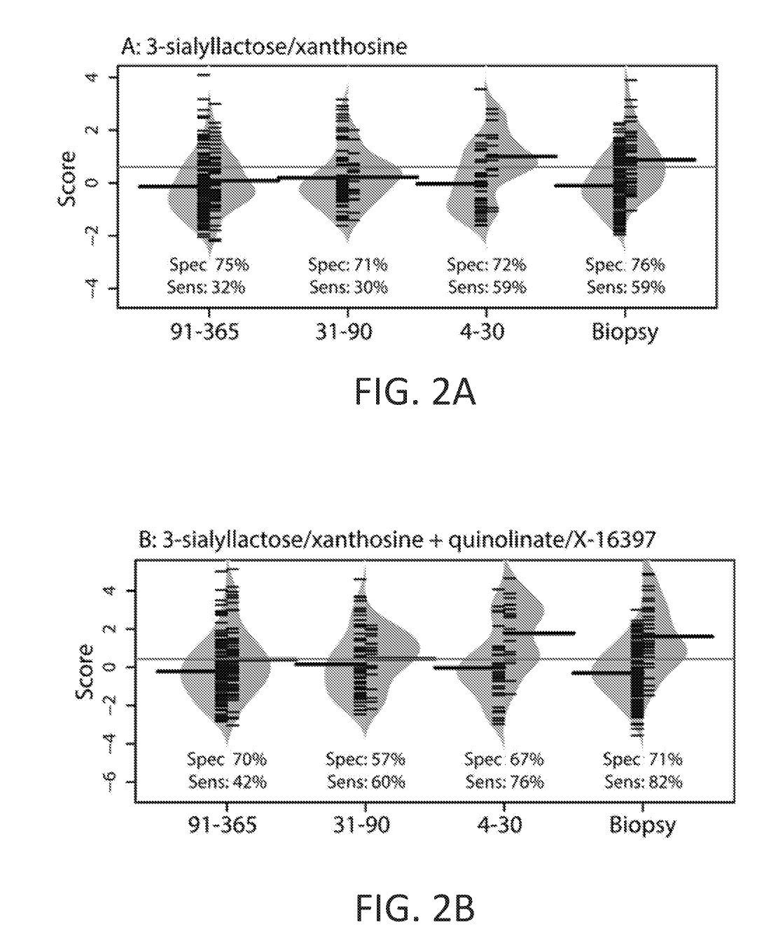

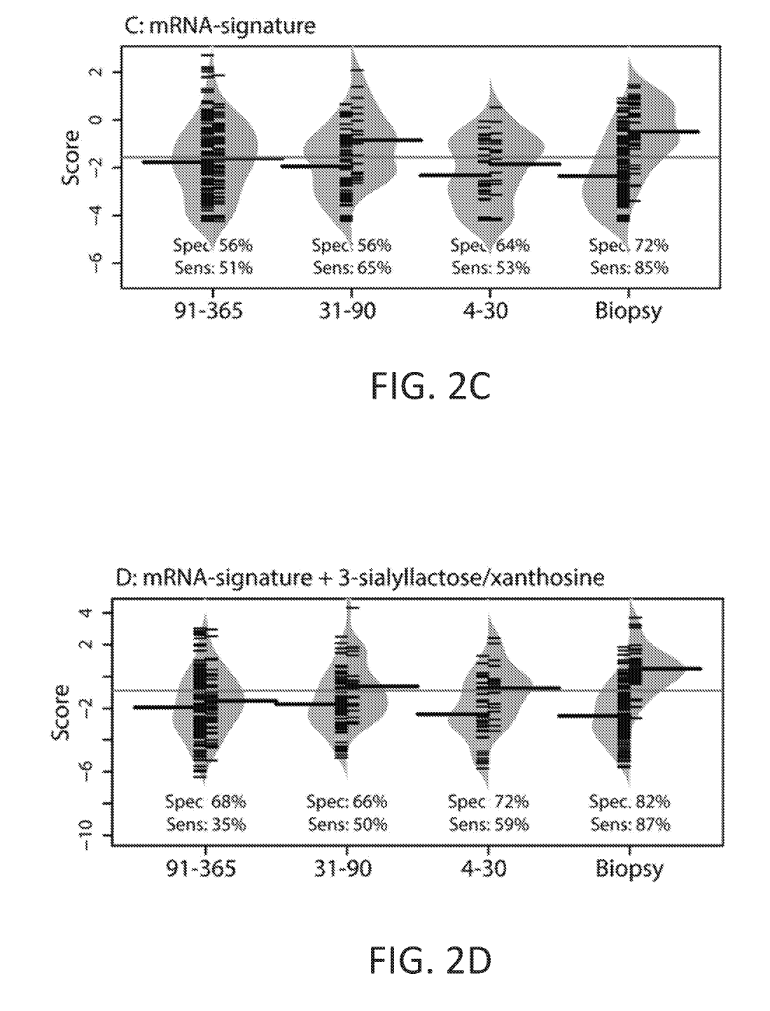

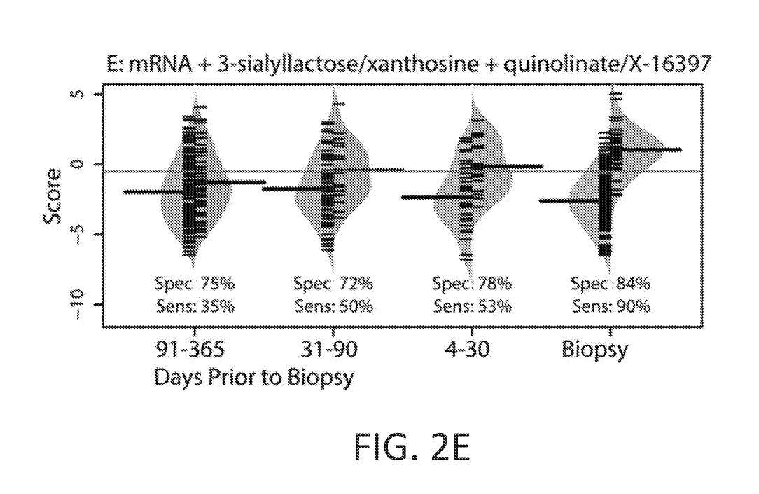

Data from this analysis are illustrated as bean plots (FIG. 2A-2E). This kind of data representation was selected over box-and-whisker plots as it presents individual data points as one dimensional scatter plots as well as represents the distribution of data points by the density shapes. See, Peter, Journal of Statistical Software 28: 1-9 (2008). The Youden cut-off of the respective signature for the distinguishing ACR biopsies from No Rejection biopsies in biopsy matched urine samples was used to calculate the sensitivity and the specificity of the signature at indicated time intervals and are included in FIG. 2A-2E.

The ratio of 3SL/X in urine samples collected during 4 days to 30 days prior to biopsy predicted future development of an ACR with a specificity of 72% and a sensitivity of 59% (FIG. 2A). The combination signature of ratios of 3SL/X and quinolinate to X-16397 in urine collected during the same time interval predicted future ACR (FIG. 2B), and the RNA signature did not outperform either of the two-metabolite signatures (FIG. 2C). A combination of the RNA signature and the 3SL/X metabolite signature (FIG. 2D) or a combination of the RNA signature and the 3SL/X and quinolinate/X-16397 metabolite signatures in urine collected up to 30 days before a biopsy had the highest specificity but not the highest sensitivity for predicting future ACR (FIG. 2E). Note that the urine samples analyzed for their prognostic ability were pristine in the sense that they were not included in the initial step that led to the construction of the diagnostic metabolite or RNA signatures and that these predictions are thus free of model bias.

### Example 7: Longitudinal Analysis in Clinically Stable Patients

A total of 385 sequential urine samples with both metabolite and RNA data were analyzed to investigate the characteristics of the signatures in the first year of transplantation in clinically stable patients (see the legend to FIG. 1 and Example 1, which specify the criteria used to classify patients as clinically stable patients and the rationale for the selection of 40 clinically stable patients for the longitudinal analysis). Data from the analysis of sequential urine samples, visualized as bean plots, show that the signatures are remarkably stable when measured in urine samples collected 30 days after kidney transplantation (FIG. 3A-3E). The signatures' ability to predict No Rejection biopsies (specificity) progressively increased over time for all signatures and a combination of the metabolite signatures and the RNA signature performed best with specificity reaching 90% in urine samples collected during post-transplant days 271 to 365. It is noteworthy that none of the urine samples included in this longitudinal analysis were included in the initial construction of the diagnostic metabolite or RNA signatures, and that the predictions shown in FIG. 3A-3E are therefore also free of model bias.

### Example 8: Targeted Assay Development

For clinical application, a high-throughput assay was developed using robotic solid-phase (RapidFire 365) extraction and Quadrupole Time-of-Flight (QTOF) MS/MS for simultaneous absolute quantification of urinary 3-sialyllactose and xanthosine levels—metabolites suggested by our non-targeted metabolomics to offer diagnostic and prognostic information regarding ACR. Notably, this novel platform allows for a theoretical urinary sample throughput of over 5000 samples daily. Because metabolite-profiling results found that ACR diagnosis may be best afforded by considering the ratio of 3SL/X, rather than absolute metabolite levels, this platform was optimized for high-throughput ratiometric assay for these analytes (see, Example 1 and FIGS. 2A-2C). To test the utility of this ratiometric approach, two or three repeated measurements of 3SL/X ratios were made for each of the 43 ACR biopsy-matched samples and 163 No Rejection biopsy-matched samples using a fresh, not previously thawed aliquot of urine supernatant. The P value for the association of the ratio of 3SL/X for discriminating ACR biopsies from No Rejection biopsies was P=4.0×10⁻⁸ with the targeted RapidFire assay, in accord with the P value obtained following analysis of non-targeted metabolomics data. The observed correlation between the non-targeted Metabolon data and targeted RapidFire-QTOF data was 0.65 (Pearson R) and the Bland-Altman method for comparison (Bland, Lancet 1: 307-310 (1986)) showed that only 12 samples (6%) were beyond the 95% limit of agreement. A targeted assay was not developed for measuring quinolinate/X-16397.

### REFERENCES

1. Suthanthiran, M, Schwartz, J E, Ding, R, Abecassis, M, Dadhania, D, Samstein, B, Knechtle, S J, Friedewald, J, Becker, Y T, Sharma, V K, Williams, N M, Chang, C S, Hoang, C, Muthukumar, T, August, P, Keslar, K S, Fairchild, R L, Hricik, D E, Heeger, P S, Han, L, Liu, J, Riggs, M, Ikle, D N, Bridges, N D, Shaked, A: Urinary-cell mRNA profile and acute cellular rejection in kidney allografts. N Engl J Med 369: 20-31, 2013.
2. Hricik, D E, Nickerson, P, Formica, R N, Poggio, E D, Rush, D, Newell, K A, Goebel, J, Gibson, I W, Fairchild, R L, Riggs, M, Spain, K, Ikle, D, Bridges, N D, Heeger, P S, consortium, C-: Multicenter validation of urinary CXCL9 as a risk-stratifying biomarker for kidney transplant injury. Am J Transplant 13: 2634-2644, 2013.
3. Bouatra, S, Aziat, F, Mandal, R, Guo, A C, Wilson, M R, Knox, C, Bjorndahl, T C, Krishnamurthy, R, Saleem, F, Liu, P, Dame, Z T, Poelzer, J, Huynh, J, Yallou, F S, Psychogios, N, Dong, E, Bogumil, R, Roehring, C, Wishart, D S: The human urine metabolome. PLoS One 8: e73076, 2013.
4. Bohra, R, Klepacki, J, Klawitter, J, Thurman, J M, Christians, U: Proteomics and metabolomics in renal transplantation-quo vadis? Transpl Int 26: 225-241, 2013.
5. Psychogios, N, Hau, D D, Peng, J, Guo, A C, Mandal, R, Bouatra, S, Sinelnikov, I, Krishnamurthy, R, Eisner, R, Gautam, B. Young, N, Xia, J, Knox, C, Dong, E, Huang, P, Hollander, Z, Pedersen, T L, Smith, S R, Bamforth, F, Greiner, R, McManus, B, Newman, J W. Goodfriend, T, Wishart, D S: The human serum metabolome. PLoS One 6: e16957, 2011.
6. Nicholson, J K, Lindon, J C: Systems biology: Metabonomics. Nature 455: 1054-1056, 2008.
7. Fiehn, O: Metabolomics—the link between genotypes and phenotypes. Plant Mol Biol 48: 155-171, 2002.
8. Pauling, L, Robinson, A B, Teranishi, R, Cary, P: Quantitative analysis of urine vapor and breath by gas-liquid partition chromatography. Proc Natl Acad Sci USA 68: 2374-2376, 1971.
9. Suhre, K, Shin, S Y, Petersen, A K, Mohney, R P, Meredith, D, Wagele, B, Altmaier, E, Deloukas, P, Erdmann, J. Grundberg, E, Hammond, C J, de Angelis, M H, Kastenmuller, G, Kottgen, A, Kronenberg, F, Mangino, M, Meisinger, C, Meitinger, T, Mewes, H W, Milburn, M V, Prehn, C, Raffler, J, Ried, J S, Romisch-Margl, W, Samani, N J, Small, K S, Wichmann, H E, Zhai, G, Illig, T, Spector, T D, Adamski, J, Soranzo, N, Gieger, C, Assimes, T L, Holm, H, Kathiresan, S, Konig, I R, McPherson, R, Reilly, M P, Roberts, R, Schunkert, H, Stewart, A F: Human metabolic individuality in biomedical and pharmaceutical research. Nature 477: 54-60, 2011.
10. Gall, W E, Beebe, K, Lawton, K A, Adam, K P, Mitchell, M W, Nakhle, P J, Ryals, J A, Milburn, M V, Nannipieri, M, Camastra, S, Natali, A, Ferrannini, E, Group, R S: alpha-hydroxybutyrate is an early biomarker of insulin resistance and glucose intolerance in a nondiabetic population. PLoS One 5: e10883, 2010.
11. Sreekumar, A, Poisson, L M, Rajendiran, T M, Khan, A P. Cao, Q, Yu. J, Laxman, B, Mehra, R, Lonigro, R J, Li, Y, Nyati, M K, Ahsan, A, Kalyana-Sundaram, S, Han, B, Cao, X, Byun, J. Omenn, G S, Ghosh, D, Pennathur, S, Alexander, D C, Berger, A, Shuster, J R, Wei, J T, Varambally, S, Beecher, C, Chinnaiyan, A M: Metabolomic profiles delineate potential role for sarcosine in prostate cancer progression. Nature 457: 910-914, 2009.
12. Spratlin, J L. Serkova, N J, Eckhardt, S G: Clinical applications of metabolomics in oncology: a review. Clin Cancer Res 15: 431-440, 2009.
13, Benjamini Y, H Y: Controlling the false discovery rate—a practical and powerful approach to multiple testing. J R Stat Soc Series B Methodol 57: 289-300, 1995.
14. Petersen, A K, Krumsiek, J, Wagele, B, Theis, F J, Wichmann, H E, Gieger, C, Suhre, K: On the hypothesis-free testing of metabolite ratios in genome-wide and metabolome-wide association studies. BMC Bioinformatics 13: 120, 2012.
15. Le, C T: A solution for the most basic optimization problem associated with an ROC curve. Stat Methods Med Res 15: 571-584, 2006.
16. Peter, K: Beanplot: A Boxplot Alternative for Visual Comparison of Distributions. Journal of Statistical Software 28: 1-9, 2008.
17, Bland, J M, Altman, D G: Statistical methods for assessing agreement between two methods of clinical measurement. Lancet 1: 307-310, 1986.
18. Blydt-Hansen, T D, Sharma, A, Gibson, I W, Mandal, R, Wishart, D S: Urinary metabolomics for noninvasive detection of borderline and acute T cell-mediated rejection in children after kidney transplantation. Am J Transplant 14: 2339-2349, 2014.
19. Goek, O N, Prehn, C, Sekula, P, Romisch-Margl, W, Doring, A, Gieger, C, Heier, M, Koenig, W, Wang-Sattler, R. Illig, T, Suhre, K, Adamski, J, Kottgen, A, Meisinger, C: Metabolites associate with kidney function decline and incident chronic kidney disease in the general population. Nephrol Dial Transplant 28: 2131-2138, 2013.
20. Goek, O N, Doring, A, Gieger, C, Heier, M, Koenig, W, Prehn, C, Romisch-Margl, W, Wang-Sattler, R, Illig, T, Suhre, K, Sekula, P, Zhai, G, Adamski, J, Kottgen, A, Meisinger, C: Serum metabolite concentrations and decreased GFR in the general population. Am J Kidney Dis 60: 197-206, 2012.
21. Suhre, K, Romisch-Margl, W, de Angelis, M H, Adamski, J, Luippold, G, Augustin, R: Identification of a potential biomarker for FABP4 inhibition: the power of lipidomics in preclinical drug testing. J Biomol Screen 16: 467-475, 2011.
22. Kurakevich, E, Hennet, T, Hausmann, M, Rogler, G, Borsig, L: Milk oligosaccharide sialyl(alpha2,3)lactose activates intestinal CD11c+ cells through TLR4. Proc Natl Acad Sci USA 110: 17444-17449, 2013.
23. Idota, T, Kawakami, H, Murakami, Y, Sugawara, M: Inhibition of cholera toxin by human milk fractions and sialyllactose. Biosci Biotechnol Biochem 59: 417-419, 1995.
24. Izquierdo-Useros, N, Lorizate, M, Contreras, F X, Rodriguez-Plata, M T, Glass, B, Erkizia, I, Prado, J G, Casas, J, Fabrias, G, Krausslich, H G, Martinez-Picado, J: Sialyllactose in viral membrane gangliosides is a novel molecular recognition pattern for mature dendritic cell capture of HIV-1. PLoS Biol 10: e1001315, 2012.
25. Moffett, J R, Namboodiri, M A: Tryptophan and the immune response. Immunology and Cell Biology 81: 247-265, 2003.
26. Hartono, C, Muthukumar, T, Suthanthiran, M: Immunosuppressive drug therapy. Cold Spring Harb Perspect Med 3: a015487, 2013.
27. Sollinger, H W: Mycophenolate mofetil for the prevention of acute rejection in primary cadaveric renal allograft recipients. U.S. Renal Transplant Mycophenolate Mofetil Study Group. Transplantation 60: 225-232, 1995.
28. Collins, A J, Foley, R N, Chavers, B, Gilbertson, D, Herzog, C, Johansen, K, Kasiske, B, Kutner, N, Liu, J, St Peter, W, Guo, H, Gustafson, S, Heubner, B, Lamb, K, Li, S, Peng, Y, Qiu, Y, Roberts, T, Skeans, M, Snyder, J, Solid, C, Thompson, B, Wang, C, Weinhandl, E, Zaun, D, Arko, C, Chen, S C, Daniels, F, Ebben, J, Frazier, E, Hanzlik, C, Johnson, R, Sheets, D, Wang, X, Forrest, B, Constantini, E, Everson, S, Eggers, P, Agodoa, L: ‘United States Renal Data System 2011 Annual Data Report: Atlas of chronic kidney disease & end-stage renal disease in the United States. Am J Kidney Dis 59: A7, e1-420, 2012.
29. Collins, A I, Kasiske, B, Herzog, C, Chavers, B, Foley, R, Gilbertson, D, Grimm, R, Liu, J, Louis, T, Manning, W, Matas, A, McBean, M, Murray, A, St Peter, W, Xue, J, Fan, Q, Guo, H, Li, S, Roberts, T, Snyder, J, Solid, C, Wang, C, Weinhandl, E, Arko, C, Chen, S C, Dalleska, F, Daniels, F, Dunning, S, Ebben, J, Frazier, E, Johnson, R, Sheets, D, Forrest, B, Berrini, D, Constantini, E, Everson, S, Frederick, P, Eggers, P, Agodoa, L: Excerpts from the United States Renal Data System 2004 annual data report: atlas of end-stage renal disease in the United States. Am J Kidney Dis 45: A5-7, S1-280, 2005.
30. Dehaven, C D, Evans, A M, Dai, H, Lawton, K A: Organization of G C/M S and LC/MS metabolomics data into chemical libraries. J Cheminform 2: 9, 2010.
31. Racusen, L C, Colvin, R B, Solez, K, Mihatsch, M J, Halloran, P F, Campbell, P M, Cecka, M J, Cosyns, J P, Demetris, A J, Fishbein, M C, Fogo, A, Furness, P, Gibson, I W, Glotz, D, Hayry, P, Hunsickern, L, Kashgarian, M, Kerman, R, Magil, A J, Montgomery, R, Morozumi, K, Nickeleit, V, Randhawa, P, Regele, H, Seron, D, Seshan, S, Sund, S, Trpkov, K: Antibody-mediated rejection criteria—an addition to the Banff 97 classification of renal allograft rejection. Am J Transplant 3: 708-714, 2003.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements describe features of the invention.

### Statements:

1. A method comprising: (a) measuring the amount or concentration of 3-sialyllactose and xanthosine; quinolinate and X-16397; or a combination thereof in a subject's urine sample; (b) obtaining a ratio of the concentration or the amount of sialyllactose/xanthosine; quinolinate/X-16397; or a combination thereof in the urine sample; and (c) determining whether the ratio of the concentration or the amount of sialyllactose/xanthosine; quinolinate/X-16397; or a combination thereof is distinct from a control ratio of a control concentration or a control amount of sialyllactose/xanthosine; quinolinate/X-16397; or a combination thereof.
2. The method of statement 1, further comprising obtaining the subject's urine sample prior to the measuring step (a).
3. The method of statement 1 or 2, further comprising removing cells from the urine sample from the subject prior to the measuring step (a).
4. The method of any of statements 1-3, wherein determining step (c) comprises ascertaining a log ratio of 3-sialyllactose/xanthosine (Log₁₀(3-sialyllactose/xanthosine)); a log ratio of quinolinate/X-16397 (log₁₀(quinolinate/X-16397)); or a combination thereof; and identifying whether the sialyllactose/xanthosine log ratio; the quinolinate/X-16397 log ratio; or a combination thereof is detectably different from a control sialyllactose/xanthosine log ratio; a control quinolinate/X-16397 log ratio; or a combination thereof in one or more urine samples of control subjects who are not undergoing or will not undergo transplant rejection for the next 4-30 days.
5. The method of any of statements 1-4, wherein determining step (c) comprises ascertaining whether the ratio of the concentration or the amount of sialyllactose/xanthosine; quinolinate/X-16397; or a combination thereof in the urine sample is greater than a cut-off value.
6. The method of any of statements 1-5, wherein determining step (c) comprises ascertaining whether:
   (i) a log ratio of 3-sialyllactoose to xanthosine is greater than 0.45, or greater than 0.5, or equal or greater than 0.55, or equal or greater than 0.59836; or
   (ii) the following metabolite signature:

log₁₀(3-sialyllactose/xanthosine)+0.9513*log₁₀(quinolinate/X-16397)
is greater than 0.38, or greater than 0.39, or greater than 0.4, or greater than 0.4, or greater than 0.41, or equal or greater than 0.4271, or greater than 0.43, or greater than 0.44.
7. The method of any of statements 1-6, wherein dysfunction or rejection of a kidney transplant can or will be ongoing in subjects where the subjects' urinary sample has:
   (i) a log ratio of 3-sialyllactoose to xanthosine that is equal or greater than 0.5, or equal or greater than 0.55, or equal or greater than 0.59836; or
   (ii) a value calculated from the following metabolite signature:

log₁₀(3-sialyllactose/xanthosine)+0.9513*log₁₀(quinolinate/X-16397)
is greater than 0.38, or greater than 0.39, or greater than 0.4, or greater than 0.4, or greater than 0.41, or equal or greater than 0.4271, or greater than 0.43, or greater than 0.44.
8. The method of any of statements 1-7, further comprising identifying a subject for treatment when the subjects' urinary sample has:
   (i) a log ratio of 3-sialyllactoose to xanthosine that is equal or greater than 0.5, or equal or greater than 0.55, or equal or greater than 0.59836; or
   (ii) a value calculated from the following combined metabolite signature:

log₁₀(3-sialyllactose/xanthosine)+0.9513*log₁₀(quinolinate/X-16397)
is greater than 0.38, or greater than 0.39, or greater than 0.4, or greater than 0.4, or greater than 0.41, or equal or greater than 0.4271, or greater than 0.43, or greater than 0.44.
9. The method of any of statements 1-8, comprising treating a subject for allograft rejection when a urine sample from the subject has a log ratio of 3-sialyllactoose to xanthosine that is equal or greater than 0.5, or equal or greater than 0.55, or equal or greater than 0.59836; or when a urine sample from the subject has a combined metabolite signature that is greater than 0.38, or greater than 0.39, or greater than 0.4, or greater than 0.4, or greater than 0.41, or equal or greater than 0.4271, or greater than 0.43, or greater than 0.44; wherein the combined metabolite signature=log₁₀(3-sialyllactose/xanthosine)+0.9513*log₁₀(quinolinate/X-16397).
10. The method of any of statements 1-9, comprising isolating and/or purifying RNA from the urine sample.
11. The method of any of statements 1-10, further comprising isolating and/or purifying RNA from cells present in the urine sample.
12. The method of any of statements 1-11, comprising quantifying CD3c mRNA, IP-10 mRNA, 18S rRNA, or a combination thereof in the urine sample.
13. The method of any of statements 1-12, comprising hybridizing at least one probe or primer to RNA from the urine sample obtained from the subject.
14. The method of any of statements 1-13, comprising hybridizing at least one probe or primer to CDR mRNA, IP-10 mRNA, 18S rRNA, or a combination thereof from the urine sample.
15. The method of any of statements 1-14, comprising hybridizing at least one probe or primer to CDR mRNA, at least one probe or primer to IP-10 mRNA, at least one probe or primer to 18S rRNA, or a combination thereof from the urine sample.
16. The method of any of statements 1-15, comprising quantifying CD3ε mRNA, IP-10 mRNA, 18S rRNA, or a combination thereof, in the urine sample by a method comprising quantitative nucleic acid amplification reactions (e.g., quantitative polymerase chain reaction (PCR)), primer extension, Northern blot, immunoassay, immunosorbent assay (ELISA), radioimmunoassay (RIA), immunofluorimetry, immunoprecipitation, equilibrium dialysis, immunodiffusion, immunoblotting, mass spectrometry, or a combination thereof
17. The method of any of statements 10-16, comprising normalizing the amount (or copy number) of CD3ε mRNA, IP-10 mRNA, or both against the amount (or copy number) of a housekeeping gene.
18. The method of statement 17, wherein the housekeeping gene is 2-Microglobulin ((32M), Glucose-6-phosphate dehydrogenase (G6PDH), 5-aminolevulinate synthase (ALAS or ALAS 1) Hypoxanthinephophoribosyltransferase (HPRT), Porphobilinogen deaminase (PBGD), or 18S rRNA.
19. The method of statement 17 or 18, wherein the housekeeping gene is 18S rRNA.
20. The method of any of statements 17-19, wherein normalizing the amount of CD3ε mRNA comprises dividing a determined absolute urinary CD3ε mRNA copy number per microgram of total RNA from a sample by a determined absolute urinary 18S rRNA copy number per microgram of total RNA times 10⁻⁶ from the same sample:

$$Sample ’ s normalized CD 3 ɛ mRNA amount = \frac{determined absolute CD 3 ɛ copy number / μg total RNA}{\begin{matrix}determined absolute 18 S rRNA \\ copy number / μg total RNA \times {10}^{- 6}\end{matrix}} .$$

21. The method of any of statements 17-20, wherein normalizing the amount of IP-10 mRNA comprises dividing a determined absolute urinary IP-10 mRNA copy number per microgram of total RNA from a sample by a determined absolute urinary 18S rRNA copy number per microgram of total RNA times 10-6 from the same sample:

$$Sample ’ s normalized CD 3 ɛ mRNA amount = \frac{determined absolute IP \text{-} 10 copy number / μg total RNA}{\begin{matrix}determined absolute 18 S rRNA \\ copy number / μg total RNA \times {10}^{- 6}\end{matrix}} .$$

22. The method of any of statements 1-21, comprising determining whether CD3ε mRNA, IP-10 mRNA, and 18S rRNA are expressed at higher levels in cells of the urine sample than a baseline, control or reference level of urinary cell expression of CD3ε mRNA, IP-10 mRNA, and 18S rRNA.
23. The method of any of statements 12-22, identifying a subject for treatment when one or more of a CD3ε mRNA, IP-10 mRNA, and 18S rRNA expression level is at least 5%, 10%, 15%, 20%, 25%, 30%, 35%, 40%, 45%, 50%, 55%, 60%, 65%, 70%, 75%, 80%, 85%, 90%, 95%, or 100% higher in the cells of the urine sample obtained from the subject than in the baseline, control or reference level of urinary cell expression of CD3ε mRNA, IP-10 mRNA, and 18S rRNA.
24. The method of any of statements 22 or 23, wherein the baseline, control or reference level of urinary cell expression of CD3ε mRNA, IP-10 mRNA, or 18S rRNA is the respective amount of urinary cell expression of CD3ε mRNA, IP-10 mRNA, or 18S rRNA expressed by one or more healthy persons or one or more persons with a well-functioning (e.g., stable) transplanted organ.

25. The method of any of statements 22-24, wherein the baseline, control or reference level of urinary cell expression of CD3ε mRNA, IP-10 mRNA, and 18S rRNA is the respective amount of urinary cell expression of CD3ε mRNA, IP-10 mRNA, and 18S rRNA expressed by a population of healthy persons or a population of persons with a well-functioning (e.g., stable) transplanted organ.
26. The method of any of statements 1-25, comprising ascertaining a RNA diagnostic signature of developing or existing dysfunction or rejection of a kidney transplant in the subject with the following algorithm:

RNA signature=−6.1487+0.8534 log₁₀(CD3ε/18S)+0.6376 log₁₀(IP-10/18S)+1.6464 log₁₀(18S)
where:
   CD3ε refers to an absolute urinary CD3ε mRNA copy number per microgram of total RNA;
   IP-10 refers to an absolute urinary IP-10 mRNA copy number per microgram of total RNA;
   18S refers to an absolute urinary 18S rRNA copy number per microgram of total RNA times 10⁻⁶.
27. The method of statement 26, further comprising identifying a subject that has an RNA signature above −1.2, or above −1.3, or above −1.4, above −1.45, or above −1.5, or above, −1.55, or above −1.563099, or above −1.56, or above −1.65, or above −1.7.
28. The method of any of statements 26-28, further comprising informing the subject that he or she has or can develop dysfunction or rejection of a kidney transplant when the subject's RNA signature is above −1.2, or above −1.3, or above −1.4, above −1.45, or above −1.5, or above, −1.55, or above −1.563099, or above −1.56, or above −1.65, or above −1.7.
29. The method of any of statements 26-29, further comprising treating a subject that has a urine sample with an RNA signature above −1.2, or above −1.3, or above −1.4, above −1.45, or above −1.5, or above, −1.55, or above −1.563099, or above −1.56, or above −1.65, or above −1.7.
30. The method of any of statements 26-30, comprising ascertaining a composite diagnostic signature with the following algorithm:

RNA-signature+1.1164*log(3SL/X)+0.6937*log(quinolinate/X-16397).
31. The method of statement 31, further comprising identifying a subject with a urine sample that has a composite diagnostic signature above 0, or above −0.1, or above −0.2, or above −0.3, or above −0.5, or above −0.5095, or above −0.51, or above −0.52 (wherein “above” means less negative).
32. The method of statement 31 or 32, further comprising treating a subject for transplant rejection when the urine sample from the subject has a composite diagnostic signature above 0, or above −0.1, or above −0.2, or above −0.3, or above −0.5, or above −0.5095, or above −0.51, or above −0.52 (wherein “above” means less negative).
33. The method of any of statements 1-33, comprising monitoring a subject over time for developing or existing dysfunction or rejection of a kidney transplant in a subject.
34. The method of any of statements 1-34, comprising monitoring a subject over time for developing or existing dysfunction or rejection of a kidney transplant in a subject by collecting urine samples from the subject and performing the method with those urine samples.
35. The method of any of statements 1-35, comprising monitoring a subject over time and informing the subject if there is continuing change in metabolite ratios or RNA levels over time.
36. The method of any of statements 1-36, comprising informing the subject of a developing or existing dysfunction or rejection of a kidney transplant in the subject.
37. The method of any of statements 1-37, further comprising treating a developing or existing dysfunction or rejection of a kidney transplant in the subject.
38. The method of any of statements 1-38, further comprising treating the subject for acute cellular rejection of the kidney allograft.
39. The method of any of statements 1-39, further comprising treating the subject wherein treating comprises plasmapheresis, administration of an anti-rejection agent, increasing a dosage of an anti-rejection agent that the subject is receiving or any combination thereof
40. The method of statement 40, wherein the anti-rejection agent is azathioprine, cyclosporine, FK506, tacrolimus, mycophenolate mofetil, anti-CD25 antibody, antithymocyte globulin, rapamycin, ACE inhibitors, perillyl alcohol, anti-CTLA4 antibody, anti-CD40L antibody, anti-thrombin III, tissue plasminogen activator, antioxidants, anti-CD 154, anti-CD3 antibody, thymoglobin, OKT3, corticosteroid, or a combination thereof
41. A method for analysis of a urine sample from a subject with a kidney transplant comprising:
   (a) assaying the urine sample to measure an absolute urinary CD3ε mRNA copy number per microgram of total RNA in the urine sample, an absolute urinary IP-10 mRNA copy number per microgram of total RNA in the urine sample, and an absolute urinary 18S rRNA copy number per microgram of total RNA times 10⁻⁶ in the urine sample;
   (b) assaying to measure a ratio of 3-sialyllactose to xanthosine in the urine sample;
   (c) assaying to measure a ratio of quinolinate to X-16397 in the urine sample;
   (d) determining an RNA signature=−6.1487+0.8534 log₁₀(CD3ε/18S)+0.6376 log₁₀(IP-10/18S)+1.6464 log₁₀(18S);
   (e) determining a metabolite signature=1.1164*log(3-sialyllactose/xanthosine)+0.6937*log(quinolinate/X-16397); and/or
   (f) identifying a composite diagnostic signature=RNA-signature+1.1164*log(3SL/X)+0.6937*log(quinolinate/X-16397);
   where:
   CD3ε is an absolute urinary CD3ε mRNA copy number per microgram of total RNA in the urine sample;
   IP-10 is an absolute urinary IP-10 mRNA copy number per microgram of total RNA in the urine sample; and
   18S is an absolute urinary 18S rRNA copy number per microgram of total RNA in the urine sample times 10⁻⁶;
   to thereby analyze the urine sample from the subject with the kidney transplant.
42. The method of statement 42, further comprising informing the subject of the combined diagnostic signature.
43. The method of statement 42 or 43, further comprising treating the subject for allograft rejection.
44. The method of any of statements 42-44, further comprising treating the subject for allograft rejection when a urine sample from the subject has a combined diagnostic signature score greater than −0.2, or greater than −0.3, or greater than −0.4, or greater than −0.45, or greater than −0.5, or greater than −0.5095 (where greater than means less negative).
45. A method comprising treating a subject for allograft rejection when a urine sample from the subject has a combined diagnostic signature greater than −0.2, or greater than −0.3, or greater than −0.4, or greater than −0.45, or greater than −0.46, or greater than −0.47, or greater than −0.48, or greater than −0.49, or greater than −0.5, or greater than −0.5095, or greater than −0.51, or greater than −0.52 (where greater than means less negative); wherein

the combined diagnostic signature=mRNA-signature+1.1164*log(3SL/X)+0.6937*log(quinolinate/X-16397); and

the RNA signature=−6.1487+0.8534 log₁₀(CD3ε/18S)+0.6376 log₁₀(IP-10/18S)+1.6464 log₁₀(18S).

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a polypeptide” includes a plurality of such nucleic acids or polypeptides (for example, a solution of metabolites, RNA, or polypeptides or a series of metabolite, RNA, or polypeptide preparations), and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The following claims describe some aspects of the invention.

1. A method for analysis of a urine sample from a subject with a kidney transplant comprising:
(a) assaying to measure a ratio of 3-sialyllactose xanthosine in the urine sample;
(b) assaying to measure a ratio of quinolinate to X-6397 in the urine sample; and/or
determining combined metabolite signature=1.1164*log(3-sialyllactose/xanthosine)+0.6937*log(quinolinate/X-16397), to thereby analyze the urine sample from the subject with the kidney transplant.

2. The method of claim 1, further comprising informing the subject of the combined metabolite signature.

3. The method of claim 1, further comprising treating the subject for allograft rejection.

4. The method of claim 1, further comprising treating the subject for allograft rejection when a log ratio of 3-sialyllactoose to xanthosine is greater than 0.55, or is equal or greater than 0.59836.

5. The method of claim 1, further comprising treating a subject for allograft rejection when a urine sample from the subject has a combined metabolite signature greater than 0.4, or is equal to or greater than 0.4271.

6. A method for analysis of a urine sample from a subject with a kidney transplant comprising:
(a) assaying the urine sample to measure an absolute urinary CD3ε mRNA copy number per microgram of total RNA in the urine sample, an absolute urinary IP-10 mRNA copy number per microgram of total RNA in the urine sample, and an absolute urinary 18S rRNA copy number per microgram of total RNA times 10⁻⁶ in the urine sample;
(b) assaying to measure a ratio of 3-sialyllactose to xanthosine in the urine sample;
(c) assaying to measure a ratio of quinolinate to X-16397 in the urine sample;
(d) determining an RNA signature=−6.1487+0.8534 log₁₀(CD3ε/18S)+0.6376 log₁₀(IP-10/18S)+1.6464 log₁₀(18S);
(e) determining a metabolite signature=1.1164*log(3-sialyllactose/xanthosine)+0.6937*log(quinolinate/X-16397); and/or
(f) identifying a composite diagnostic signature=RNA-signature+1.1164*log(3SL/X)+0.6937*log(quinolinate/X-16397);
where:
CD3ε is an absolute urinary CDR mRNA copy number per microgram of total RNA in the urine sample;
IP-10 is an absolute urinary IP-10 mRNA copy number per microgram of total RNA in the urine sample; and
18S is an absolute urinary 18S rRNA copy number per microgram of total RNA in the urine sample times 10⁻⁶;
to thereby analyze the urine sample from the subject with the kidney transplant.

7. The method of claim 6, further comprising informing the subject of the composite diagnostic signature.

8. The method of claim 6, further comprising treating the subject for allograft rejection.

9. The method of claim 6, further comprising treating the subject for allograft rejection when the urine sample has a combined diagnostic signature score above −0.45, or above −0.5, or equal to or above −0.5095.

10. A method comprising treating a subject for allograft rejection when a urine sample from the subject has a composite diagnostic signature above −0.45, or above −0.5, or equal to or above −0.5095; wherein:
he composite diagnostic signature=mRNA-signature+164*log(3SL/X)+0.6937*log(quinolinate/X-16397); and
the RNA signature=−6.1487+0.8534 log₁₀(CD3ε/18S)+0.6376 log₁₀(IP-10/18S)+1.6464 log₁₀(18S).


SEQUENCE LISTING (3 sequences)

SEQ ID NO: 1 - DNA, 2235 bp, Homo sapiens
  misc_feature (1)...(2235): n = A,T,C or G
cgctgctcctcccgtcgccgtccgggcccgtccgtccgtccgtccgtcgtcctcctcgctnnnncggggcgccgggcccgtcctcacnggcccccgnnnnngtccnggcccgtcggggcctcgccgcgctctaccttacctacctggttgatcctgccagtagcatatgcttgtctcaaagattaagccatgcatgtctaagtacgcacggccggtacagtgaaactgcgaatggctcattaaatcagttatggttcctttggtcgctcgctcctctcctacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattccttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcattaacggagcccggacggcggcccgcggcggcgccgcgccgcgcttccctccgcacacccacccccccaccgcgacggcgcgtgcgggcggggccgtgcccgttcgttcgctcgctcgttcgttcgccgcccggcccggccgcgagagccgagaactcgggagggagacgggggagagagagagagagagagagagagagagagagagagagagaaagaagggcgtgt

SEQ ID NO: 2 - DNA, 1534 bp, Homo sapiens
tattgtcagagtcctcttgtttggccttctaggaaggctgtgggacccagctttcttcaaccagtccaggtggaggcctctgccttgaacgtttccaagtgaggtaaaacccgcaggcccagaggcctctctacttcctgtgtggggttcagaaaccctcctcccctcccagcctcaggtgcctgcttcagaaaatgaagtagtaagtctgctggcctccgccatcttagtaaagtaacagtcccatgaaacaaagatgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgaccctctggagaacactgcctcccgctggcccaggtctcctctccagtccccctgcgactccctgtttcctgggctagtcttggaccccacgagagagaatcgttcctcagcctcatggtgaactcgcgccctccagcctgatcccccgctccctcctccctgccttctctgctggtacccagtcctaaaatattgctgcttcctcttcctttgaagcatcatcagtagtcacaccctcacagctggcctgccctcttgccaggatatttatttgtgctattcactcccttccctttggatgtaacttctccgttcagttccctccttttcttgcatgtaagttgtcccccatcccaaagtattccatctacttttctatcgccgtccccttttgcagccctctctggggatggactgggtaaatgttgacagaggccctgccccgttcacagatcctggccctgagccagccctgtgctcctccctcccccaacactccctaccaaccccctaatcccctactccctccaccccccctccactgtaggccactggatggtcatttgcatctccgtaaatgtgctctgctcctcagctgagagagaaaaaaataaactgtatttggctgcaagaaaaaaaaaaaaaaaaaaaaaa

SEQ ID NO: 3 - DNA, 1172 bp, Homo sapiens
gagacattcctcaattgcttagacatattctgagcctacagcagaggaacctccagtctcagcaccatgaatcaaactgcgattctgatttgctgccttatctttctgactctaagtggcattcaaggagtacctctctctagaaccgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaatgtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtataagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaataaaaatgaggtactctcctggaaatattaagaaagactatctaaatgttgaaagatcaaaaggttaataaagtaattataact